(19) United States
(12) Patent Application Publication
(10) Pub. No.: US 2025/0345284 A1
(43) Pub. Date: Nov. 13, 2025

(54) LIPID NANOPARTICLE (LNP) COMPOSITIONS AND METHODS OF USE THEREOF

(71) Applicant: The Trustees Of The University of Pennsylvania, Philadelphia, PA (US)

(72) Inventors: Michael Mitchell, Philadelphia, PA (US); Savan Patel, Boston, MA (US); Margaret M. Billingsley, Hockessin, DE (US); Xuexiang Han, Philadelphia, PA (US); Hanwen Zhang, Philadelphia, PA (US)

(73) Assignee: The Trustees Of The University of Pennsylvania, Philadelphia, PA (US)

(21) Appl. No.: 18/695,471

(22) PCT Filed: Sep. 30, 2022
(86) PCT No.: PCT/US2022/077346

§ 371 Date: Mar. 26, 2024

Related U.S. Application Data
(60) Provisional application No. 63/251,255, filed on Oct. 1, 2021.
(60) Provisional application No. 63/290,220, filed on Dec. 16, 2021.

(51) Int. Cl. A61K 9/51; A61K 31/7105; A61K 38/46; A61K 47/22; A61K 47/28; A61K 47/64; A61K 47/69

(52) U.S. Cl. CPC A61K 9/5123; A61K 9/5146; A61K 31/7105; A61K 38/465; A61K 47/22; A61K 47/28; A61K 47/6425; A61K 47/6925

(57) ABSTRACT

The present disclosure relates, in part, to lipid nanoparticles (LNPs) comprising cholesterol substitutes (i.e., cholesterol analogs and/or derivatives) and methods of use thereof for in vivo delivery of nucleic acid molecules and/or therapeutic agents to a target cell. In certain embodiments, the nucleic acid molecules encode chimeric antigen receptors (CARs). In certain embodiments, the target cell is a T cell. In certain embodiments, the LNPs of the present disclosure are anti-inflammatory. In certain embodiments, the present disclosure relates to the use of the LNPs described herein for the treatment, prevention, and/or amelioration of diseases and/or disorders in a subject, including but not limited to cancer.

### CROSS-REFERENCE TO RELATED APPLICATIONS

This application claims priority under 35 U.S.C. § 119 (e) to U.S. Provisional Patent Application No. 63/290,220, filed Dec. 16, 2021, and U.S. Provisional Patent Application No. 63/251,255, filed Oct. 1, 2021, both of which applications are incorporated herein by reference in their entireties.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

Over the past decade, immunotherapy has become a critical tool in the medical treatment of a variety of diseases and conditions. These therapies target immune cells (e.g., T cells, B cells, and dendritic cells, inter alia) which are a part of a complex set of biological networks that control the body's response to cancers, foreign pathogens, and other stimuli. Immunotherapies span a wide range of modalities from antibody-based inhibitors to genetically-engineered immune cells. Messenger RNA-based immunotherapy, one particularly modality, has piqued significant interest due to the transient nature of messenger RNA (mRNA) and decreased risk of genomic integration that is associated with DNA. Ionizable lipid nanoparticles (LNPs) are the most clinical advanced non-viral delivery platform for RNA therapeutics, as illustrated by the clinical success of Onpattro and the Pfizer/BioNTech and Moderna COVID-19 mRNA vaccines. Ionizable lipids can protect and deliver mRNA therapeutics to target cells by overcoming biological barriers.

However, the LNP/mRNA complex can interact with the innate immune system and trigger immune responses. While mRNAs can be modified to be immune-silent, the LNPs themselves have been shown to induce strong inflammatory responses in immune cells. LNPs can activate the immune system by interacting with pattern recognition receptors (PRRs) on antigen presenting cells (APCs), such as toll-like receptors (TLRs). Previous studies have shown that the interaction of LNPs with PPRs will subsequently trigger the release of pro-inflammatory cytokines such as tumor necrosis factor alpha (TNF-α), suggesting the general onset of innate immune response. The inflammatory responses can then reduce the translation efficiency of mRNA and provoke immune-related adverse effects. Therefore, premedication with anti-inflammatory drugs and anti-histamines is needed for LNP-based mRNA therapeutics in the clinic.

Further, the prominent technology for mRNA delivery to immune cells is electroporation, which is a method whereby cell membranes are permeabilized via electric pulses to allow for the transduction of mRNA into the cytosol. However, electroporation of cells in ex vivo settings tends to be highly toxic to target cells. Furthermore, electroporation is limited to ex vivo applications, making it challenging to translate mRNA immunotherapies to in vivo platforms.

Thus, there is a need in the art for LNPs which can both can suppress unwanted innate immune responses and deliver mRNA cargo potently with low toxicity to immune cells in a manner which enables in vivo applications. The present disclosure addresses these needs.

### BRIEF SUMMARY

In one aspect, the present disclosure provides a lipid nanoparticle (LNP). In certain embodiments, the LNP comprises at least one ionizable lipid, wherein the ionizable lipid comprises about 10 mol % to about 50 mol % of the LNP. In certain embodiments, the LNP comprises at least one helper lipid, wherein the helper lipid comprises about 10 mol % to about 45 mol % of the LNP. In certain embodiments, the LNP comprises at least one selected from the group consisting of cholesterol and a cholesterol-substitute, wherein the combination of the cholesterol and cholesterol-substitute comprise about 5 mol % to about 50 mol % of the LNP. In certain embodiments, the LNP comprises at least one polyethylene glycol (PEG) or PEG-conjugated lipid, wherein the PEG or PEG conjugated lipid comprises about 0.5 mol % to about 12.5 mol % of the LNP.

In certain embodiments, the LNP further comprises at least one selected from the group consisting of a nucleic acid molecule and a therapeutic agent. In certain embodiments, the nucleic acid is mRNA.

In certain embodiments, the cholesterol-substitute is dexamethasone.

In certain embodiments, the cholesterol-substitute is selected from the group consisting of 7-α-hydroxycholesterol, 7-β-hydroxycholesterol, 19-hydroxycholesterol, 20-(S)-hydroxycholesterol, 24-(S)-hydroxycholesterol, 25-hydroxycholesterol, 7-ketocholesterol, 5,6-epoxycholesterol, 3β,5α, 6β-trihydroxycholesterol, 4β-hydroxycholesterol, 27-hydroxycholesterol and 22-(R)-hydroxycholesterol.

In certain embodiments, the cholesterol-substitute is selected from the group consisting of chenodeoxycholic acid (CDCA), cholic acid (CA), deoxycholic acid (DCA), lithocholic acid (LCA), taurocholic acid, glycocholic acid, taurochenodeoxycholic acid, and glycochenodeoxycholic acid.

In another aspect, the present disclosure provides a pharmaceutical composition comprising at least one LNP of the present disclosure and a pharmaceutically acceptable carrier. In certain embodiments, the pharmaceutical composition further comprises an adjuvant. In certain embodiments, the pharmaceutical composition is a vaccine.

In another aspect, the present disclosure provides a method of delivering at least one selected from the group consisting of a nucleic acid molecule and a therapeutic agent to a target cell in a subject in need thereof. In certain embodiments, the method comprises administering to the subject a therapeutically effectively amount of at least one LNP of the present disclosure and/or at least one pharmaceutical composition of the present disclosure. In certain embodiments, the method treats, prevents, and/or ameliorates at least one selected from the group consisting of a viral infection, a bacterial infection, a fungal infection, a parasitic infection, cancer, or a disease or disorder associated with cancer.

### BRIEF DESCRIPTION OF THE FIGURES

The drawings illustrate generally, by way of example, but not by way of limitation, various embodiments of the present application.

FIGS. 1A-1B depict the chemical structures of cholesterol and dexamethasone and schematic illustration of anti-inflammatory LNP to reduce adverse effects and improve mRNA transfection. FIG. 1A depicts the chemical structures of cholesterol (left, MW: 386.65 g/mol) and dexamethasone (right, MW: 392.47 g/mol). FIG. 1B depicts exemplary data demonstrating that anti-inflammatory LNPs suppress the local inflammation caused by LNPs in immune cells leading to reduced adverse effects and enhanced hepatic mRNA transfection. LNPs are proposed to stimulate immune cells (e.g., macrophages). In certain embodiments, dexamethasone (i.e., Dex) can reduce the release of proinflammatory cytokines (e.g., TNF-α), and thus improve hepatic transfection and minimize the adverse effects of LNPs.

FIG. 2 depicts an exemplary diagram showing the formulation of Dex-incorporated LNPs via microfluidic mixing. In certain embodiments, mRNA is dissolved in an aqueous phase while a PEG-conjugated lipid (e.g., C14PEG-2000), MC3, DSPC, cholesterol, and dexamethasone are dissolved in an organic phase. In certain embodiments, the two solutions are rapidly mixed in a microfluidic device to form mRNA-LNPs.

FIGS. 3A-3B depict the hydrodynamic size of non-limiting LNPs of the present disclosure as measured by Dynamic Light Scattering (DLS). FIG. 3A depicts an intensity-based size distribution of C10D0 LNP. FIG. 3B depicts an intensity-based size distribution of C9D1 LNP. Three representative technical replicate results for each LNP are shown.

FIGS. 4A-4C depict exemplary experimental data demonstrating in vitro luciferase expression and cell viability in HepG2 cells, and TNF-α levels in RAW246.7 cells, following treatment with exemplary mRNA LNPs of the present disclosure incorporating Dex. FIG. 4A depicts in vitro luciferase mRNA transfection in HepG2 cells 24 hours after treatment. FIG. 4B depicts cell viability of HepG2 cells 24 hours after treatment. FIG. 4C depicts the TNF-α production in RAW246.7 cells 24 hours after treatment. Data is presented as mean±SD (n=3). n.s., non-significant, ***P<0.001.

FIGS. 5A-5B depict exemplary experimental data demonstrating in vitro luciferase expression and cell viability in HepG2 cells following treatment with exemplary LNPs of the present disclosure formulated with different cholesterol:dexamethasone (C:D) ratios. FIG. 5A depicts in vitro luciferase mRNA transfection in HepG2 cells 24 hours post-treatment. FIG. 5B depicts cell viability of HepG2 cells 24 hours post-treatment. Data is presented as mean±SD (n=3).

FIGS. 6A-6B depict exemplary experimental data demonstrating in vivo TNF-α levels and mRNA delivery following intravenous administration of C10D0 and C9D1 LNPs. FIG. 6A depicts serum TNF-α levels following treatment with C10D0 or C9D1 LNPs in mice. Serum was collected 20 hours after treatment. Data is presented as mean±SD (n=3). *P<0.05. b). FIG. 6B depicts in vivo luciferase expression; for each mouse, 4 μg of LNP-formulated luciferase mRNA was administered intravenously.

FIGS. 7A-7C depicts the motivation, design, and synthesis of non-limiting lipid nanoparticles (LNP) with hydroxycholesterol substitution. FIG. 7A provides a schematic depicting LNP components, formulation, and generic expected structure. FIG. 7B provides a diagram depicting LNP delivery into a T cell and endosomal trafficking mechanisms involving the Rab family of proteins. In certain embodiments, Rab5, Rab7, and Rab11 associate with the early, late, and recycling endosomes, respectively. FIG. 7C depicts the design of a non-limiting LNP library incorporating the substitution of various hydroxycholesterols with at least a portion of unmodified cholesterol.

FIGS. 8A-8C depict exemplary experimental data demonstrating the characterization and stability of non-limiting exemplary LNP formulations of the present disclosure comprising cholesterol analogs. FIG. 8A depicts structures of six hydroxycholesterols (e.g., 7α-HC, 78-HC, 19-HC, 20 (S)-HC, 24 (S)-HC, and 25-HC) grouped by the location of the hydroxyl modification on the cholesterol molecule. LNPs comprising cholesterol modified at any position of the polycyclic core (i.e., the “body” of the molecule) are denoted by “A” (i.e., A1, A2, A3) and LNPs containing cholesterol modifications to alkyl chain substituent of the 5-membered ring of cholesterol (i.e., the “tail” of the molecule) are denoted by “B” (i.e., B1, B2, B3). FIG. 8B depicts measurements of z-average diameter, PDI, mRNA concentration, and encapsulation efficiency for S2 LNPs and LNP formulations with 100% cholesterol substitution taken over 28 days to assess LNP stability. The sample DLS curves show representative size distributions of LNP formulations S2 and A1-100 at day 3. n=3. Error bars denote standard deviation. FIG. 8C depicts the pKa, zeta potential, z-average diameter, and PDI measurements for the exemplary LNPs of the present disclosure. n=3. Error bars denote standard deviation.

FIGS. 9A-9B depict a screen of LNP library for luciferase mRNA delivery (FIG. 9A) and viability (FIG. 9B) in a T cell line (Jurkats) to identify top formulations. Jurkat cells were treated with LNP formulations at 60 ng mRNA/60,000 cells for 24 h. Luciferase expression was normalized to cells treated with a standard LNP formulation (S2), and background luminescence was subtracted. Percent viability of cells treated with LNPs was determined by normalization to untreated cells. Legend denotes percent substitution of each hydroxycholesterol substitute into the S2 formulation. n=3 biological replicates. Error bars denote standard deviation. An ANOVA was used to determine if treatment group means differed significantly. **: p<0.01 in Tukey's honest significance test between LNP candidate and S2.

FIGS. 10A-10C depict exemplary experimental data demonstrating a screen of LNPs formulated with top-performing hydroxycholesterol substitutes in primary human T cells. FIG. 10A depicts the luciferase expression in primary human T cells treated with LNP formulations containing A1, A2, or B1 hydroxycholesterols or S2 at a dose of 300 ng mRNA/60,000 cells for 24 hours. n=3 biological replicates. Error bars denote standard deviation. An ANOVA was performed to determine if treatment group means differed significantly. *: p<0.05 in student t-test between LNP candidate and S2. FIGS. 10B-10C depicts the luciferase expression (FIG. 10B) and relative viability (FIG. 10C) of primary human T cells treated with S2, A1-25, and A1-50 at various doses. Luciferase expression was normalized to cells treated with the standard LNP formulation (S2), and background luminescence was subtracted. Percent viability was determined by normalizing to untreated cells. Legend denotes percent substitution of each hydroxycholesterol substitute into the S2 formulation. Each patient is represented by a different marker. n=3 biological replicates. Error bars denote standard deviation. *: p<0.05, **: p<0.01 in student t-test between from S2 and either A1-25 or A1-50.

FIG. 11 depicts exemplary experimental data demonstrating endosomal uptake and colocalization of LNPs with endosomes in Jurkats. Confocal microscopy images of Jurkat cells treated with DiO-labeled LNPs at 60 ng mRNA/60,000 cells and stained with Lysotracker. Images were merged, background was subtracted, and Spearman's rank-order correlation was used to quantify association between LNPs and acidic organelles in cells. Colocalization statistics (i.e., Spearman's rank-order correlation) were obtained from 5 fields of view (at least 90 cells in total) of each treatment group. Error bars denote standard deviation. An ANOVA was performed to determine if group means differed significantly. *: p<0.05 in student t-test with Bonferroni p-value correction between colocalization statistics from S2 and either A1-25 or A1-50.

FIGS. 12A-12B depict exemplary experimental data demonstrating the characterization of LNP endosomal trafficking. Confocal microscopy images of Jurkats stained with antibodies for Rab5, Rab7, or Rab11. Cells were either untreated (UT) or treated with S2, A1-25, or A1-50 at 60 ng mRNA/60,000 cells (FIG. 12A) or 150 ng mRNA/60,000 cells (FIG. 12B). Rab5, Rab7, and Rab11 expression was quantified by averaging fluorescent signal from at least 50 cells in each treatment group. Expression of Rab proteins was normalized to untreated cells. Error bars denote standard deviation. An ANOVA was used within each Rab protein group to determine if group means differed significantly. *: p<0.05, **: p<0.01 in student t-test with Bonferroni p-value correction between S2 and either A1-25 or A1-50.

FIG. 13 provides the chemical structure of exemplary cholesterol analogs (e.g., bile acids) used as components in non-limiting LNP-comprising compositions of the present disclosure.

FIGS. 14A-14E provide bar graphs depicting exemplary experimental data relating to mRNA (e.g., luciferase) delivery and/or expression with administration of exemplary LNPs of the present disclosure comprising chenodeoxycholic acid (CDCA), cholic acid (CA), deoxycholic acid (DCA), and lithocholic acid (LCA) in Caco-2 (FIG. 14A), HeLa (FIG. 14B), HepG2 (FIG. 14C), Jurkat (FIG. 14D), and Raji (FIG. 14E) cell lines; percentages indicate the percentage of cholesterol analog comprising the cholesterol component of the LNP (e.g., 25% for CDCA indicates 25% of the total cholesterol component of the LNP comprises CDCA and 75% is cholesterol).

FIG. 15A-15B depicts exemplary data relating to mRNA (e.g., luciferase) delivery and/or expression in the liver, spleen, uterus, stomach, small intestine, and large intestine of mice with intraperitoneal (i.p.) administration of selected LNPs of the present disclosure comprising cholesterol analogs, as a bar graph (FIG. 15A) and imaging (FIG. 15B).

FIGS. 16A-16B depict exemplary data relating to mRNA (e.g., luciferase) delivery and/or expression in the spleen and liver with intraperitoneal (i.p.) administration of selected LNPs of the present disclosure comprising cholesterol analogs, as a bar graph (FIG. 16A) and imaging (FIG. 16B).

FIG. 17A-17E provide a bar graph (FIG. 17A) and images (FIG. 17B-17E) depicting mRNA (e.g., luciferase) delivery and/or expression in the heart, lungs, kidneys, uterus, stomach, small intestine, and large intestine of mice with intravenous administration of selected LNPs of the present disclosure comprising cholesterol analogs, including S2 (FIG. 17B), C100 (FIG. 17C), D50 (FIG. 17D), and E75 (FIG. 17E).

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms.

Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group) 3 wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, C18 alkyl chains, ether linkages between the head group and alkyl chains, and 0 to 3 double bonds. Such lipids include, e.g., DSDMA, DLinDMA, DLenDMA, and DODMA.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).e in the animal's state of health.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C2-heteroaryl can be a 5-ring with two carbon atoms and three heteroatoms, a 6-ring with two carbon atoms and four heteroatoms and so forth. Likewise a C4-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C2-heterocyclyl can be a 5-ring with two carbon atoms and three heteroatoms, a 6-ring with two carbon atoms and four heteroatoms and so forth. Likewise a C4-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

“Homologous” as used herein, refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKa of the protonatable group in the range of about 4 to about 7.

“Immunogen” refers to any substance introduced into the body in order to generate an immune response. That substance can a physical molecule, such as a protein, or can be encoded by a vector, such as DNA, mRNA, or a virus.

“Immune cell,” as the term is used herein, means any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl psuedouridien, or another modified nucleoside.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27:196-197).

In certain embodiments, “pseudouridine” refers, In some embodiments, to m¹acp³Ψ (1-methyl-3-(3-amino-3-carboxypropyl) pseudouridine. In some embodiments, the term refers to m¹Ψ (1-methylpseudouridine). In some embodiments, the term refers to Ψm (2′-O-methylpseudouridine. In some embodiments, the term refers to m⁵D (5-methyldihydrouridine). In some embodiments, the term refers to m³Ψ (3-methylpseudouridine). In some embodiments, the term refers to a pseudouridine moiety that is not further modified. In some embodiments, the term refers to a monophosphate, diphosphate, or triphosphate of any of the above pseudouridines. In some embodiments, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo (carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Lipid Nanoparticles (LNPs) and Compositions Thereof

In one aspect, the present disclosure provides a lipid nanoparticle (LNP), which in certain embodiments is part of a composition, such as but not limited to a pharmaceutical composition.

In certain embodiments, the LNP comprises at least one ionizable lipid, wherein the ionizable lipid comprises about 10 mol % to about 50 mol % of the LNP. In certain embodiments, the LNP comprises at least one helper lipid, wherein the helper lipid comprises about 10 mol % to about 45 mol % of the LNP. In certain embodiments, the LNP comprises at least one selected from the group consisting of cholesterol and a cholesterol-substitute, wherein the combination of the cholesterol and cholesterol-substitute comprise about 5 mol % to about 50 mol % of the LNP. In certain embodiments, the LNP comprises at least one polyethylene glycol (PEG) or PEG-conjugated lipid, wherein the PEG or PEG conjugated lipid comprises about 0.5 mol % to about 12.5 mol % of the LNP.

In certain embodiments, the LNP further comprises at least one selected from the group consisting of a nucleic acid molecule and a therapeutic agent. In certain embodiments, the nucleic acid and/or therapeutic agent is at least partially encapsulated therein.

In certain embodiments, the LNP further comprises at least one agent selected from the group consisting of an mRNA, a siRNA, a microRNA, a CRISPR-Cas9, a small molecule, a protein, and an antibody. In certain embodiments, the LNP comprises a nucleic acid molecule. In certain embodiments, the nucleic acid molecule is a DNA molecule or an RNA molecule. In certain embodiments, the nucleic acid molecule is selected from the group consisting of cDNA, mRNA, miRNA, siRNA, modified RNA, antagomir, antisense molecule, and a targeted nucleic acid, or any combination thereof. In certain embodiments, the nucleic acid molecule encodes a chimeric antigen receptor (CAR). In certain embodiments, the CAR is specific for binding to a surface antigen of a pathogenic cell or a tumor cell. In certain embodiments, the LNP further comprises a targeting domain specific for binding to a target cell of interest. In certain embodiments, the target cell is selected from the group consisting of a peripheral blood mononuclear cell and an immune cell. In certain embodiments, the LNP comprises an immune cell targeting domain specific for binding to a T cell.

In certain embodiments, the targeting domain specifically binds to at least one surface molecule selected from the group consisting of CD1, CD2, CD3, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7.

In certain embodiments, the ionizable lipid is a compound of Formula (I), or a salt or solvate thereof:

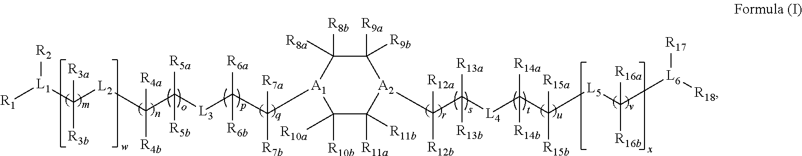

wherein:
A₁ and A₂ is independently selected from the group consisting of CH, N, and P;
L₁ and L₆ are each independently selected from the group consisting of CR₁₉ and N;
each occurrence of L₂ and L₅ is independently selected from the group consisting of —CH₂—, —CHR₁₉—, —O—, —NH—, and —NR₁₉—;
L₃ and L₄ are each independently selected from the group consisting of —CH₂—, —CHR₁₉—, —O—, —NH—, and —NR₁₉—;
each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆ₐ, R_{16b}, R₁₇, R₁₈, and R₁₉ is independently selected from the group consisting of H, halogen, optionally substituted C₁-C₂₈ alkyl, optionally substituted C₃-C₁₂ cycloalkyl, —Y(R₂₀)_{z′}(R₂₁)_{z″}-(optionally substituted C₃-C₁₂ cycloalkyl, optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₈ alkenyl, optionally substituted C₅-C₁₂ cycloalkenyl, —Y(R₂₀)_{z′}(R₂₁)_{z″}-(optionally substituted C₅-C₁₂ cycloalkenyl), optionally substituted C₂-C₂₈ alkynyl, optionally substituted C₆-C₁₂ cycloalkynyl, —Y(R₂₀)_{z′}(R₂₁)_{z″}-(optionally substituted C₆-C₁₂ cycloalkynyl), optionally substituted C₆-C₁₀ aryl, —Y(R₂₀)_{z′}(R₂₁)_{z″}-(optionally substituted C₆-C₁₀ aryl), optionally substituted C₂-C₁₂ heteroaryl, —Y(R₂₀)_{z′}(R₂₁)_{z″}-(optionally substituted C₂-C₁₂ heteroaryl), C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —Y(R₂₀)_{z′}(R₂₁)_{z″}-ester, —Y(R₂₀)_{z′}(R₂₁)_{z″}, —NO₂, —CN, and sulfoxy,
   or two geminal substituents selected from R₃ₐ and R_{3b}, R₄ₐ and R_{4b}, R₅ₐ and R_{5b}, R₆ₐ, and R_{6b}, R₇ₐ and R_{7b}, R₈ₐ and R_{8b}, R₉ₐ and R_{9b}, R₁₀ₐ and R_{10b}, R₁₁ₐ and R_{11b}, R₁₂ₐ and R_{12b}, R₁₃ₐ and R_{13b}, R₁₄ₐ and R_{14b}, or R₁₅ₐ and R_{15b} can combine with the C atom to which they are bound to form C═O;
each occurrence of Y is independently selected from the group consisting of C, N, O, S, and P;
each occurrence of R₂₀ and R₂₁ is independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —NO₂, —CN, and sulfoxy,
   or R₂₀ and R₂₁ can combine with the Y atom to which they are bound to form a C═O);
each occurrence of z′ and z″ is independently 0, 1, or 2; and
each occurrence of m, n, o, p, q, r, s, t, u, v, w, and x are is independently 0, 1, 2; 3, 4, or 5.

In certain embodiments, the ionizable lipid of Formula (I) is selected from the group consisting of:

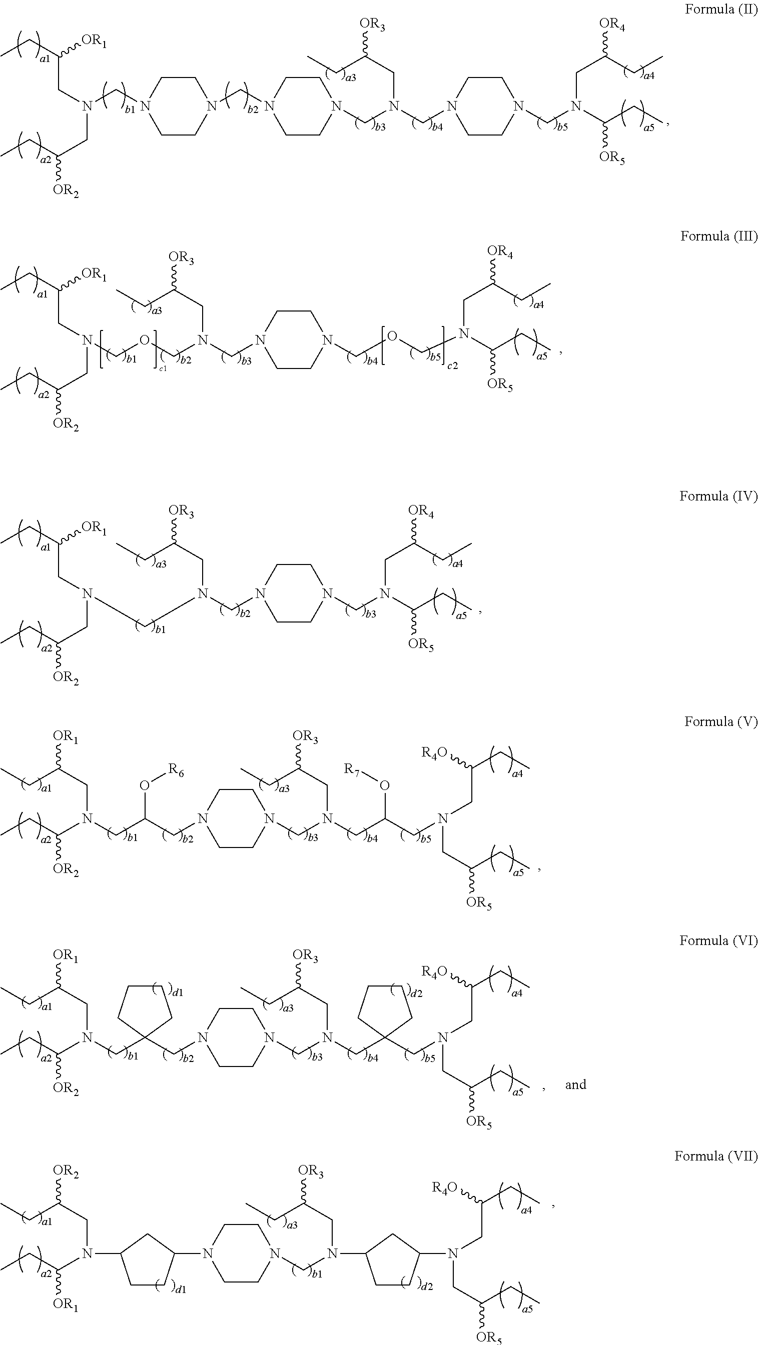

wherein:
R₁, R₂, R₃, R₄, R₅, R₆, and R₇ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester;
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25;
b¹, b², b³, b⁴, and b⁵ are each independently 0, 1, 2, 3, 4, or 5;
c¹ and c² are each independently 0, 1, 2, 3, 4, or 5; and
d¹ and d² are each independently 0, 1, 2, 3, 4, or 5.

In certain embodiments, the ionizable lipid of Formula (I) is selected from the group consisting of:

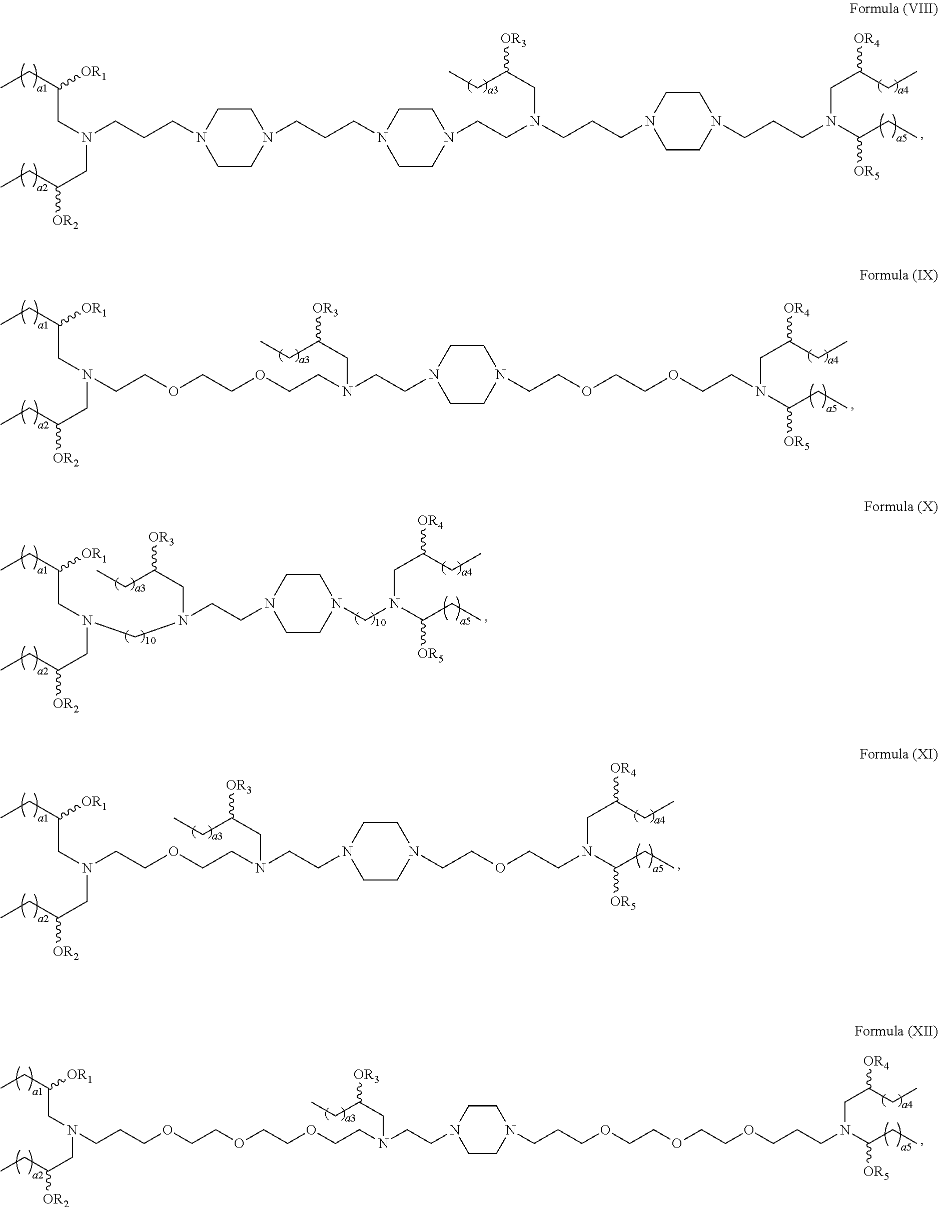

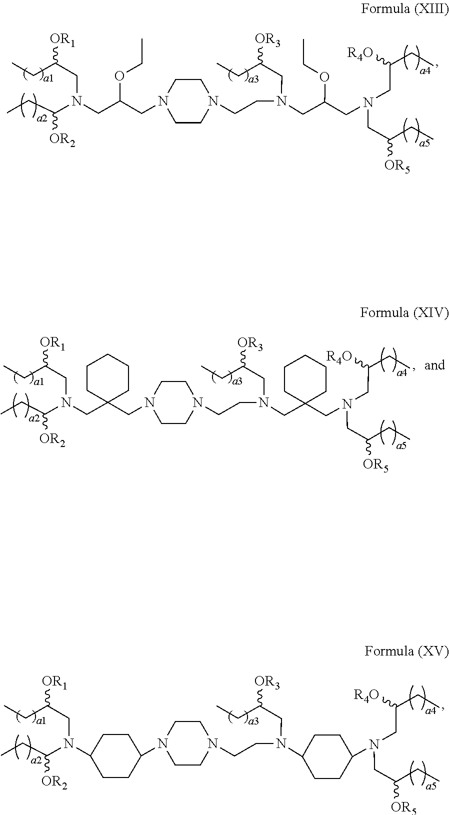

wherein:
R₁, R₂, R₃, R₄, and R₅ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester; and
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25.

In certain embodiments, the ionizable lipid of Formula (I) comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol):

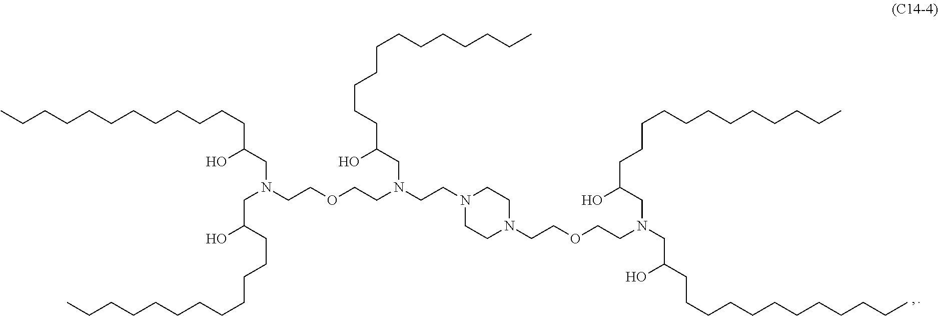 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

In certain embodiments, cholesterol-substitute is dexamethasone.

In certain embodiments, the cholesterol and/or cholesterol-substitute comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the cholesterol and/or cholesterol-substitute comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the cholesterol and/or cholesterol-substitute comprise about 38.5 mol % of the LNP. In certain embodiments, the cholesterol and/or cholesterol-substitute comprise less than about 38.5 mol % of the LNP. In certain embodiments, the cholesterol and/or cholesterol-substitute comprise more than about 38.5 mol % of the LNP.

In certain embodiments, the cholesterol and cholesterol-substitute have a weight ratio of about 10:1 (cholesterol:dexamethasone). In certain embodiments, the cholesterol and cholesterol-substitute have a weight ratio of about 10:2 (cholesterol:dexamethasone). In certain embodiments, the cholesterol and cholesterol-substitute have a weight ratio of about 10:3 (cholesterol:dexamethasone). In certain embodiments, the cholesterol and cholesterol-substitute have a weight ratio of about 10:4 (cholesterol:dexamethasone). In certain embodiments, the cholesterol and cholesterol-substitute have a weight ratio of about 10:5 (cholesterol:dexamethasone). In certain embodiments, the cholesterol and cholesterol-substitute have a weight ratio of about 10:6 (cholesterol:dexamethasone). In certain embodiments, the cholesterol and cholesterol-substitute have a weight ratio of about 10:7 (cholesterol:dexamethasone). In certain embodiments, the cholesterol and cholesterol-substitute have a weight ratio of about 10:8 (cholesterol:dexamethasone). In certain embodiments, the cholesterol and cholesterol-substitute have a weight ratio of about 10:9 (cholesterol:dexamethasone). In certain embodiments, the cholesterol and cholesterol-substitute have a weight ratio of about 1:1 (cholesterol:dexamethasone). In certain embodiments, the cholesterol and cholesterol-substitute have a weight ratio of about 1:10 (cholesterol:dexamethasone). In certain embodiments, the cholesterol and cholesterol-substitute have a weight ratio of about 2:10 (cholesterol:dexamethasone). In certain embodiments, the cholesterol and cholesterol-substitute have a weight ratio of about 3:10 (cholesterol:dexamethasone). In certain embodiments, the cholesterol and cholesterol-substitute have a weight ratio of about 4:10 (cholesterol:dexamethasone). In certain embodiments, the cholesterol and cholesterol-substitute have a weight ratio of about 5:10 (cholesterol:dexamethasone). In certain embodiments, the cholesterol and cholesterol-substitute have a weight ratio of about 6:10 (cholesterol:dexamethasone). In certain embodiments, the cholesterol and cholesterol-substitute have a weight ratio of about 7:10 (cholesterol:dexamethasone). In certain embodiments, the cholesterol and cholesterol-substitute have a weight ratio of about 8:10 (cholesterol:dexamethasone). In certain embodiments, the cholesterol and cholesterol-substitute have a weight ratio of about 9:10 (cholesterol:dexamethasone). In certain embodiments, the cholesterol and cholesterol-substitute have a weight ratio of about 8:2 (cholesterol:dexamethasone). In certain embodiments, the cholesterol and cholesterol-substitute have a weight ratio of about 7:3 (cholesterol:dexamethasone).

In certain embodiments, the cholesterol and cholesterol-substitute have a weight ratio of less than about 10:1 (cholesterol:dexamethasone). In certain embodiments, the cholesterol and cholesterol-substitute have a weight ratio of less than about 10:2 (cholesterol:dexamethasone). In certain embodiments, the cholesterol and cholesterol-substitute have a weight ratio of less than about 10:3 (cholesterol:dexamethasone). In certain embodiments, the cholesterol and cholesterol-substitute have a weight ratio of less than about 10:4 (cholesterol:dexamethasone). In certain embodiments, the cholesterol and cholesterol-substitute have a weight ratio of less than about 10:5 (cholesterol:dexamethasone). In certain embodiments, the cholesterol and cholesterol-substitute have a weight ratio of less than about 10:6 (cholesterol:dexamethasone). In certain embodiments, the cholesterol and cholesterol-substitute have a weight ratio of less than about 10:7 (cholesterol:dexamethasone). In certain embodiments, the cholesterol and cholesterol-substitute have a weight ratio of less than about 10:8 (cholesterol:dexamethasone). In certain embodiments, the cholesterol and cholesterol-substitute have a weight ratio of less than about 10:9 (cholesterol:dexamethasone). In certain embodiments, the cholesterol and cholesterol-substitute have a weight ratio of less than about 1:1 (cholesterol:dexamethasone). In certain embodiments, the cholesterol and cholesterol-substitute have a weight ratio of less than about 1:10 (cholesterol:dexamethasone). In certain embodiments, the cholesterol and cholesterol-substitute have a weight ratio of less than about 2:10 (cholesterol:dexamethasone). In certain embodiments, the cholesterol and cholesterol-substitute have a weight ratio of less than about 3:10 (cholesterol:dexamethasone). In certain embodiments, the cholesterol and cholesterol-substitute have a weight ratio of less than about 4:10 (cholesterol:dexamethasone). In certain embodiments, the cholesterol and cholesterol-substitute have a weight ratio of less than about 5:10 (cholesterol:dexamethasone). In certain embodiments, the cholesterol and cholesterol-substitute have a weight ratio of less than about 6:10 (cholesterol:dexamethasone). In certain embodiments, the cholesterol and cholesterol-substitute have a weight ratio of less than about 7:10 (cholesterol:dexamethasone). In certain embodiments, the cholesterol and cholesterol-substitute have a weight ratio of less than about 8:10 (cholesterol:dexamethasone). In certain embodiments, the cholesterol and cholesterol-substitute have a weight ratio of less than about 9:10 (cholesterol:dexamethasone). In certain embodiments, the cholesterol and cholesterol-substitute have a weight ratio of less than about 8:2 (cholesterol:dexamethasone). In certain embodiments, the cholesterol and cholesterol-substitute have a weight ratio of less than about 7:3 (cholesterol:dexamethasone).

In certain embodiments, the cholesterol and cholesterol-substitute have a weight ratio of more than about 10:1 (cholesterol:dexamethasone). In certain embodiments, the cholesterol and cholesterol-substitute have a weight ratio of more than about 10:2 (cholesterol:dexamethasone). In certain embodiments, the cholesterol and cholesterol-substitute have a weight ratio of more than about 10:3 (cholesterol:dexamethasone). In certain embodiments, the cholesterol and cholesterol-substitute have a weight ratio of more than about 10:4 (cholesterol:dexamethasone). In certain embodiments, the cholesterol and cholesterol-substitute have a weight ratio of more than about 10:5 (cholesterol:dexamethasone). In certain embodiments, the cholesterol and cholesterol-substitute have a weight ratio of more than about 10:6 (cholesterol:dexamethasone). In certain embodiments, the cholesterol and cholesterol-substitute have a weight ratio of more than about 10:7 (cholesterol:dexamethasone). In certain embodiments, the cholesterol and cholesterol-substitute have a weight ratio of more than about 10:8 (cholesterol:dexamethasone). In certain embodiments, the cholesterol and cholesterol-substitute have a weight ratio of more than about 10:9 (cholesterol:dexamethasone). In certain embodiments, the cholesterol and cholesterol-substitute have a weight ratio of more than about 1:1 (cholesterol:dexamethasone). In certain embodiments, the cholesterol and cholesterol-substitute have a weight ratio of more than about 1:10 (cholesterol:dexamethasone). In certain embodiments, the cholesterol and cholesterol-substitute have a weight ratio of more than about 2:10 (cholesterol:dexamethasone). In certain embodiments, the cholesterol and cholesterol-substitute have a weight ratio of more than about 3:10 (cholesterol:dexamethasone). In certain embodiments, the cholesterol and cholesterol-substitute have a weight ratio of more than about 4:10 (cholesterol:dexamethasone). In certain embodiments, the cholesterol and cholesterol-substitute have a weight ratio of more than about 5:10 (cholesterol:dexamethasone). In certain embodiments, the cholesterol and cholesterol-substitute have a weight ratio of more than about 6:10 (cholesterol:dexamethasone). In certain embodiments, the cholesterol and cholesterol-substitute have a weight ratio of more than about 7:10 (cholesterol:dexamethasone). In certain embodiments, the cholesterol and cholesterol-substitute have a weight ratio of more than about 8:10 (cholesterol:dexamethasone). In certain embodiments, the cholesterol and cholesterol-substitute have a weight ratio of more than about 9:10 (cholesterol:dexamethasone). In certain embodiments, the cholesterol and cholesterol-substitute have a weight ratio of more than about 8:2 (cholesterol:dexamethasone). In certain embodiments, the cholesterol and cholesterol-substitute have a weight ratio of more than about 7:3 (cholesterol:dexamethasone).

In certain embodiments, the ionizable lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the ionizable lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mol % of the LNP. In certain embodiments, the ionizable lipid comprises about 50 mol % of the LNP. In certain embodiments, the ionizable lipid comprises less than about 50 mol % of the LNP. In certain embodiments, the ionizable lipid comprises more than about 50 mol % of the LNP.

In certain embodiments, the at least one ionizable lipid comprises MC3. In certain embodiments, the at least one ionizable lipid comprises C12-200. In certain embodiments, the ionizable lipid comprises C14-494.

In certain embodiments, the helper lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the helper lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the helper lipid comprises about 10 mol % of the LNP. In certain embodiments, the helper lipid comprises less than about 10 mol % of the LNP. In certain embodiments, the helper lipid comprises more than about 10 mol % of the LNP.

In certain embodiments, the helper lipid is 1,2-distearoyl-sn-glycero-3-phosphoethanolamine (DSPC).

In certain embodiments, the PEG or PEG-conjugated lipid comprises about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the PEG or PEG-conjugated lipid comprises about 0.5, 0.6, 0.7, 0.8, 0.9, 1, 2, 3, 4, 5, 6, 7, 8, 9, 10, 11, 12, or about 13 mol % of the LNP. In certain embodiments, the PEG or PEG-conjugated lipid comprises about 1.5 mol % of the LNP. In certain embodiments, the PEG or PEG-conjugated lipid comprises less than about 1.5 mol % of the LNP. In certain embodiments, the PEG or PEG-conjugated lipid comprises more than about 1.5 mol % of the LNP.

In certain embodiments, the PEG or PEG-conjugated lipid comprises 1,2-dimyristoyl-sn-glycero-3-phosphoethanolamine-N-(methoxy(polyethyleneglycol)-2000) (C14PEG-2000).

In certain embodiments, the molar ratio of (a):(b):(c):(d) in the LNP is about 50:10:38.5:1.5.

In certain embodiments, the cholesterol-substitute is a hydroxy substituted cholesterol. In certain embodiments, the cholesterol-substitute is an epoxy substituted cholesterol. In certain embodiments, the cholesterol-substitute is a keto substituted cholesterol.

In certain embodiments, the cholesterol and/or cholesterol-substitute comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the cholesterol and/or cholesterol-substitute comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the cholesterol and/or cholesterol-substitute comprise about 46.5 mol % of the LNP. In certain embodiments, the cholesterol and/or cholesterol-substitute comprise less than about 46.5 mol % of the LNP. In certain embodiments, the cholesterol and/or cholesterol-substitute comprise more than about 46.5 mol % of the LNP.

In certain embodiments, the cholesterol-substitute is 7-α-hydroxycholesterol. In certain embodiments, the cholesterol-substitute is 7-β-hydroxycholesterol. In certain embodiments, the cholesterol-substitute is 19-hydroxycholesterol. In certain embodiments, the cholesterol-substitute is 20-(S)-hydroxycholesterol. In certain embodiments, the cholesterol-substitute is 24-(S)-hydroxycholesterol. In certain embodiments, the cholesterol-substitute is 25-hydroxycholesterol. In certain embodiments, the cholesterol-substitute is 7-ketocholesterol. In certain embodiments, the cholesterol-substitute is 5,6-epoxycholesterol. In certain embodiments, the cholesterol-substitute is 3β, 5α, 6β-trihydroxycholesterol. In certain embodiments, the cholesterol-substitute is 4β-hydroxycholesterol. In certain embodiments, the cholesterol-substitute is 27-hydroxycholesterol. In certain embodiments, the cholesterol-substitute is 22-(R)-hydroxycholesterol.

In certain embodiments, the cholesterol and cholesterol-substitute have a molar percentage ratio of about 0:100 (cholesterol:cholesterol-substitute). In certain embodiments, the cholesterol and cholesterol-substitute have a molar percentage ratio of about 5:95 (cholesterol:cholesterol-substitute). In certain embodiments, the cholesterol and cholesterol-substitute have a molar percentage ratio of about 10:90 (cholesterol:cholesterol-substitute). In certain embodiments, the cholesterol and cholesterol-substitute have a molar percentage ratio of about 12.5:87.5 (cholesterol:cholesterol-substitute). In certain embodiments, the cholesterol and cholesterol-substitute have a molar percentage ratio of about 15:85 (cholesterol:cholesterol-substitute). In certain embodiments, the cholesterol and cholesterol-substitute have a molar percentage ratio of about 20:80 (cholesterol:cholesterol-substitute). In certain embodiments, the cholesterol and cholesterol-substitute have a molar percentage ratio of about 25:75 (cholesterol:cholesterol-substitute). In certain embodiments, the cholesterol and cholesterol-substitute have a molar percentage ratio of about 30:70 (cholesterol:cholesterol-substitute). In certain embodiments, the cholesterol and cholesterol-substitute have a molar percentage ratio of about 35:65 (cholesterol:cholesterol-substitute). In certain embodiments, the cholesterol and cholesterol-substitute have a molar percentage ratio of about 40:60 (cholesterol:cholesterol-substitute). In certain embodiments, the cholesterol and cholesterol-substitute have a molar percentage ratio of about 45:55 (cholesterol:cholesterol-substitute). In certain embodiments, the cholesterol and cholesterol-substitute have a molar percentage ratio of about 50:50 (cholesterol:cholesterol-substitute). In certain embodiments, the cholesterol and cholesterol-substitute have a molar percentage ratio of about 55:45 (cholesterol:cholesterol-substitute). In certain embodiments, the cholesterol and cholesterol-substitute have a molar percentage ratio of about 60:40 (cholesterol:cholesterol-substitute). In certain embodiments, the cholesterol and cholesterol-substitute have a molar percentage ratio of about 65:35 (cholesterol:cholesterol-substitute). In certain embodiments, the cholesterol and cholesterol-substitute have a molar percentage ratio of about 70:30 (cholesterol:cholesterol-substitute). In certain embodiments, the cholesterol and cholesterol-substitute have a molar percentage ratio of about 75:25 (cholesterol:cholesterol-substitute). In certain embodiments, the cholesterol and cholesterol-substitute have a molar percentage ratio of about 80:20 (cholesterol:cholesterol-substitute). In certain embodiments, the cholesterol and cholesterol-substitute have a molar percentage ratio of about 85:15 (cholesterol:cholesterol-substitute). In certain embodiments, the cholesterol and cholesterol-substitute have a molar percentage ratio of about 87.5:12.5 (cholesterol:cholesterol-substitute). In certain embodiments, the cholesterol and cholesterol-substitute have a molar percentage ratio of about 90:10 (cholesterol:cholesterol-substitute). In certain embodiments, the cholesterol and cholesterol-substitute have a molar percentage ratio of about 95:5 (cholesterol:cholesterol-substitute). In certain embodiments, the cholesterol and cholesterol-substitute have a molar percentage ratio of about 100:0 (cholesterol:cholesterol-substitute).

In certain embodiments, the cholesterol and cholesterol-substitute have a molar percentage ratio of less than about 0:100 (cholesterol:cholesterol-substitute). In certain embodiments, the cholesterol and cholesterol-substitute have a molar percentage ratio of less than about 5:95 (cholesterol:cholesterol-substitute). In certain embodiments, the cholesterol and cholesterol-substitute have a molar percentage ratio of less than about 10:90 (cholesterol:cholesterol-substitute). In certain embodiments, the cholesterol and cholesterol-substitute have a molar percentage ratio of less than about 12.5:87.5 (cholesterol:cholesterol-substitute). In certain embodiments, the cholesterol and cholesterol-substitute have a molar percentage ratio of less than about 15:85 (cholesterol:cholesterol-substitute). In certain embodiments, the cholesterol and cholesterol-substitute have a molar percentage ratio of less than about 20:80 (cholesterol:cholesterol-substitute). In certain embodiments, the cholesterol and cholesterol-substitute have a molar percentage ratio of less than about 25:75 (cholesterol:cholesterol-substitute). In certain embodiments, the cholesterol and cholesterol-substitute have a molar percentage ratio of less than about 30:70 (cholesterol:cholesterol-substitute). In certain embodiments, the cholesterol and cholesterol-substitute have a molar percentage ratio of less than about 35:65 (cholesterol:cholesterol-substitute). In certain embodiments, the cholesterol and cholesterol-substitute have a molar percentage ratio of less than about 40:60 (cholesterol:cholesterol-substitute). In certain embodiments, the cholesterol and cholesterol-substitute have a molar percentage ratio of less than about 45:55 (cholesterol:cholesterol-substitute). In certain embodiments, the cholesterol and cholesterol-substitute have a molar percentage ratio of less than about 50:50 (cholesterol:cholesterol-substitute). In certain embodiments, the cholesterol and cholesterol-substitute have a molar percentage ratio of less than about 55:45 (cholesterol:cholesterol-substitute). In certain embodiments, the cholesterol and cholesterol-substitute have a molar percentage ratio of less than about 60:40 (cholesterol:cholesterol-substitute). In certain embodiments, the cholesterol and cholesterol-substitute have a molar percentage ratio of less than about 65:35 (cholesterol:cholesterol-substitute). In certain embodiments, the cholesterol and cholesterol-substitute have a molar percentage ratio of less than about 70:30 (cholesterol:cholesterol-substitute). In certain embodiments, the cholesterol and cholesterol-substitute have a molar percentage ratio of less than about 75:25 (cholesterol:cholesterol-substitute). In certain embodiments, the cholesterol and cholesterol-substitute have a molar percentage ratio of less than about 80:20 (cholesterol:cholesterol-substitute). In certain embodiments, the cholesterol and cholesterol-substitute have a molar percentage ratio of less than about 85:15 (cholesterol:cholesterol-substitute). In certain embodiments, the cholesterol and cholesterol-substitute have a molar percentage ratio of less than about 87.5:12.5 (cholesterol:cholesterol-substitute). In certain embodiments, the cholesterol and cholesterol-substitute have a molar percentage ratio of less than about 90:10 (cholesterol:cholesterol-substitute). In certain embodiments, the cholesterol and cholesterol-substitute have a molar percentage ratio of less than about 95:5 (cholesterol:cholesterol-substitute). In certain embodiments, the cholesterol and cholesterol-substitute have a molar percentage ratio of less than about 100:0 (cholesterol:cholesterol-substitute).

In certain embodiments, the cholesterol and cholesterol-substitute have a molar percentage ratio of more than about 0:100 (cholesterol:cholesterol-substitute). In certain embodiments, the cholesterol and cholesterol-substitute have a molar percentage ratio of more than about 5:95 (cholesterol:cholesterol-substitute). In certain embodiments, the cholesterol and cholesterol-substitute have a molar percentage ratio of more than about 10:90 (cholesterol:cholesterol-substitute). In certain embodiments, the cholesterol and cholesterol-substitute have a molar percentage ratio of more than about 12.5:87.5 (cholesterol:cholesterol-substitute). In certain embodiments, the cholesterol and cholesterol-substitute have a molar percentage ratio of more than about 15:85 (cholesterol:cholesterol-substitute). In certain embodiments, the cholesterol and cholesterol-substitute have a molar percentage ratio of more than about 20:80 (cholesterol:cholesterol-substitute). In certain embodiments, the cholesterol and cholesterol-substitute have a molar percentage ratio of more than about 25:75 (cholesterol:cholesterol-substitute). In certain embodiments, the cholesterol and cholesterol-substitute have a molar percentage ratio of more than about 30:70 (cholesterol:cholesterol-substitute). In certain embodiments, the cholesterol and cholesterol-substitute have a molar percentage ratio of more than about 35:65 (cholesterol:cholesterol-substitute). In certain embodiments, the cholesterol and cholesterol-substitute have a molar percentage ratio of more than about 40:60 (cholesterol:cholesterol-substitute). In certain embodiments, the cholesterol and cholesterol-substitute have a molar percentage ratio of more than about 45:55 (cholesterol:cholesterol-substitute). In certain embodiments, the cholesterol and cholesterol-substitute have a molar percentage ratio of more than about 50:50 (cholesterol:cholesterol-substitute). In certain embodiments, the cholesterol and cholesterol-substitute have a molar percentage ratio of more than about 55:45 (cholesterol:cholesterol-substitute). In certain embodiments, the cholesterol and cholesterol-substitute have a molar percentage ratio of more than about 60:40 (cholesterol:cholesterol-substitute). In certain embodiments, the cholesterol and cholesterol-substitute have a molar percentage ratio of more than about 65:35 (cholesterol:cholesterol-substitute). In certain embodiments, the cholesterol and cholesterol-substitute have a molar percentage ratio of more than about 70:30 (cholesterol:cholesterol-substitute). In certain embodiments, the cholesterol and cholesterol-substitute have a molar percentage ratio of more than about 75:25 (cholesterol:cholesterol-substitute). In certain embodiments, the cholesterol and cholesterol-substitute have a molar percentage ratio of more than about 80:20 (cholesterol:cholesterol-substitute). In certain embodiments, the cholesterol and cholesterol-substitute have a molar percentage ratio of more than about 85:15 (cholesterol:cholesterol-substitute). In certain embodiments, the cholesterol and cholesterol-substitute have a molar percentage ratio of more than about 87.5:12.5 (cholesterol:cholesterol-substitute). In certain embodiments, the cholesterol and cholesterol-substitute have a molar percentage ratio of more than about 90:10 (cholesterol:cholesterol-substitute). In certain embodiments, the cholesterol and cholesterol-substitute have a molar percentage ratio of more than about 95:5 (cholesterol:cholesterol-substitute). In certain embodiments, the cholesterol and cholesterol-substitute have a molar percentage ratio of more than about 100:0 (cholesterol:cholesterol-substitute).

In certain embodiments, the ionizable lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the ionizable lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mol % of the LNP. In certain embodiments, the ionizable lipid comprises about 30 mol % of the LNP. In certain embodiments, the ionizable lipid comprises less than about 30 mol % of the LNP. In certain embodiments, the ionizable lipid comprises more than about 30 mol % of the LNP.

In certain embodiments, the ionizable lipid is C14-494.

In certain embodiments, the helper lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the helper lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the helper lipid comprises about 16 mol % of the LNP. In certain embodiments, the helper lipid comprises less than about 16 mol % of the LNP. In certain embodiments, the helper lipid comprises more than about 16 mol % of the LNP.

In certain embodiments, the helper lipid is dioleoyl-phosphatidylethanolamine (DOPE).

In certain embodiments, the PEG or PEG-conjugated lipid comprises about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the PEG or PEG-conjugated lipid comprises about 0.5, 0.6, 0.7, 0.8, 0.9, 1, 2, 3, 4, 5, 6, 7, 8, 9, 10, 11, 12, or about 13 mol % of the LNP. In certain embodiments, the PEG or PEG-conjugated lipid comprises about 2.5 mol % of the LNP. In certain embodiments, the PEG or PEG-conjugated lipid comprises less than about 2.5 mol % of the LNP. In certain embodiments, the PEG or PEG-conjugated lipid comprises more than about 2.5 mol % of the LNP.

In certain embodiments, the PEG or PEG-conjugated lipid comprises 1,2-dimyristoyl-sn-glycero-3-phosphoethanolamine-N-(methoxy(polyethyleneglycol)-2000) (C14PEG-2000).

In certain embodiments, the molar ratio of (a):(b):(c):(d) in the LNP is about 30:16:46.5:2.5.

In certain embodiments, (c) comprises cholesterol and 7α-hydroxycholesterol, wherein the cholesterol and 7α-hydroxycholesterol have a molar ratio of about 50:50 (cholesterol: 7-α-hydroxycholesterol). In certain embodiments, (c) comprises cholesterol and 7-α-hydroxycholesterol, wherein the cholesterol and 7α-hydroxycholesterol have a molar ratio of less than about 50:50 (cholesterol: 7-α-hydroxycholesterol). In certain embodiments, (c) comprises cholesterol and 7α-hydroxycholesterol, wherein the cholesterol and 7-α-hydroxycholesterol have a molar ratio of more than about 50:50 (cholesterol: 7-α-hydroxycholesterol).

In certain embodiments, (c) comprises cholesterol and 7α-hydroxycholesterol, wherein the cholesterol and 7α-hydroxycholesterol have a molar ratio of about 75:25 (cholesterol: 7-α-hydroxycholesterol). In certain embodiments, (c) comprises cholesterol and 7α-hydroxycholesterol, wherein the cholesterol and 7α-hydroxycholesterol have a molar ratio of less than about 75:25 (cholesterol: 7-α-hydroxycholesterol). In certain embodiments, (c) comprises cholesterol and 7α-hydroxycholesterol, wherein the cholesterol and 7α-hydroxycholesterol have a molar ratio of more than about 75:25 (cholesterol: 7-α-hydroxycholesterol).

In certain embodiments, the cholesterol-substitute is a carboxy-substituted cholesterol. In certain embodiments, the cholesterol-substitute is a bile acid.

In certain embodiments, the cholesterol and/or cholesterol-substitute comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the cholesterol and/or cholesterol-substitute comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the cholesterol and/or cholesterol-substitute comprise about 46.5 mol % of the LNP. In certain embodiments, the cholesterol and/or cholesterol-substitute comprise less than about 46.5 mol % of the LNP. In certain embodiments, the cholesterol and/or cholesterol-substitute comprise more than about 46.5 mol % of the LNP.

In certain embodiments, the cholesterol and cholesterol-substitute have a molar percentage ratio of about 0:100 (cholesterol:cholesterol-substitute). In certain embodiments, the cholesterol and cholesterol-substitute have a molar percentage ratio of about 5:95 (cholesterol:cholesterol-substitute). In certain embodiments, the cholesterol and cholesterol-substitute have a molar percentage ratio of about 10:90 (cholesterol:cholesterol-substitute). In certain embodiments, the cholesterol and cholesterol-substitute have a molar percentage ratio of about 12.5:87.5 (cholesterol:cholesterol-substitute). In certain embodiments, the cholesterol and cholesterol-substitute have a molar percentage ratio of about 15:85 (cholesterol:cholesterol-substitute). In certain embodiments, the cholesterol and cholesterol-substitute have a molar percentage ratio of about 20:80 (cholesterol:cholesterol-substitute). In certain embodiments, the cholesterol and cholesterol-substitute have a molar percentage ratio of about 25:75 (cholesterol:cholesterol-substitute). In certain embodiments, the cholesterol and cholesterol-substitute have a molar percentage ratio of about 30:70 (cholesterol:cholesterol-substitute). In certain embodiments, the cholesterol and cholesterol-substitute have a molar percentage ratio of about 35:65 (cholesterol:cholesterol-substitute). In certain embodiments, the cholesterol and cholesterol-substitute have a molar percentage ratio of about 40:60 (cholesterol:cholesterol-substitute). In certain embodiments, the cholesterol and cholesterol-substitute have a molar percentage ratio of about 45:55 (cholesterol:cholesterol-substitute). In certain embodiments, the cholesterol and cholesterol-substitute have a molar percentage ratio of about 50:50 (cholesterol:cholesterol-substitute). In certain embodiments, the cholesterol and cholesterol-substitute have a molar percentage ratio of about 55:45 (cholesterol:cholesterol-substitute). In certain embodiments, the cholesterol and cholesterol-substitute have a molar percentage ratio of about 60:40 (cholesterol:cholesterol-substitute). In certain embodiments, the cholesterol and cholesterol-substitute have a molar percentage ratio of about 65:35 (cholesterol:cholesterol-substitute). In certain embodiments, the cholesterol and cholesterol-substitute have a molar percentage ratio of about 70:30 (cholesterol:cholesterol-substitute). In certain embodiments, the cholesterol and cholesterol-substitute have a molar percentage ratio of about 75:25 (cholesterol:cholesterol-substitute). In certain embodiments, the cholesterol and cholesterol-substitute have a molar percentage ratio of about 80:20 (cholesterol:cholesterol-substitute). In certain embodiments, the cholesterol and cholesterol-substitute have a molar percentage ratio of about 85:15 (cholesterol:cholesterol-substitute). In certain embodiments, the cholesterol and cholesterol-substitute have a molar percentage ratio of about 87.5:12.5 (cholesterol:cholesterol-substitute). In certain embodiments, the cholesterol and cholesterol-substitute have a molar percentage ratio of about 90:10 (cholesterol:cholesterol-substitute). In certain embodiments, the cholesterol and cholesterol-substitute have a molar percentage ratio of about 95:5 (cholesterol:cholesterol-substitute). In certain embodiments, the cholesterol and cholesterol-substitute have a molar percentage ratio of about 100:0 (cholesterol:cholesterol-substitute).

In certain embodiments, the cholesterol and cholesterol-substitute have a molar percentage ratio of less than about 0:100 (cholesterol:cholesterol-substitute). In certain embodiments, the cholesterol and cholesterol-substitute have a molar percentage ratio of less than about 5:95 (cholesterol:cholesterol-substitute). In certain embodiments, the cholesterol and cholesterol-substitute have a molar percentage ratio of less than about 10:90 (cholesterol:cholesterol-substitute). In certain embodiments, the cholesterol and cholesterol-substitute have a molar percentage ratio of less than about 12.5:87.5 (cholesterol:cholesterol-substitute). In certain embodiments, the cholesterol and cholesterol-substitute have a molar percentage ratio of less than about 15:85 (cholesterol:cholesterol-substitute). In certain embodiments, the cholesterol and cholesterol-substitute have a molar percentage ratio of less than about 20:80 (cholesterol:cholesterol-substitute). In certain embodiments, the cholesterol and cholesterol-substitute have a molar percentage ratio of less than about 25:75 (cholesterol:cholesterol-substitute). In certain embodiments, the cholesterol and cholesterol-substitute have a molar percentage ratio of less than about 30:70 (cholesterol:cholesterol-substitute). In certain embodiments, the cholesterol and cholesterol-substitute have a molar percentage ratio of less than about 35:65 (cholesterol:cholesterol-substitute). In certain embodiments, the cholesterol and cholesterol-substitute have a molar percentage ratio of less than about 40:60 (cholesterol:cholesterol-substitute). In certain embodiments, the cholesterol and cholesterol-substitute have a molar percentage ratio of less than about 45:55 (cholesterol:cholesterol-substitute). In certain embodiments, the cholesterol and cholesterol-substitute have a molar percentage ratio of less than about 50:50 (cholesterol:cholesterol-substitute). In certain embodiments, the cholesterol and cholesterol-substitute have a molar percentage ratio of less than about 55:45 (cholesterol:cholesterol-substitute). In certain embodiments, the cholesterol and cholesterol-substitute have a molar percentage ratio of less than about 60:40 (cholesterol:cholesterol-substitute). In certain embodiments, the cholesterol and cholesterol-substitute have a molar percentage ratio of less than about 65:35 (cholesterol:cholesterol-substitute). In certain embodiments, the cholesterol and cholesterol-substitute have a molar percentage ratio of less than about 70:30 (cholesterol:cholesterol-substitute). In certain embodiments, the cholesterol and cholesterol-substitute have a molar percentage ratio of less than about 75:25 (cholesterol:cholesterol-substitute). In certain embodiments, the cholesterol and cholesterol-substitute have a molar percentage ratio of less than about 80:20 (cholesterol:cholesterol-substitute). In certain embodiments, the cholesterol and cholesterol-substitute have a molar percentage ratio of less than about 85:15 (cholesterol:cholesterol-substitute). In certain embodiments, the cholesterol and cholesterol-substitute have a molar percentage ratio of less than about 87.5:12.5 (cholesterol:cholesterol-substitute). In certain embodiments, the cholesterol and cholesterol-substitute have a molar percentage ratio of less than about 90:10 (cholesterol:cholesterol-substitute). In certain embodiments, the cholesterol and cholesterol-substitute have a molar percentage ratio of less than about 95:5 (cholesterol:cholesterol-substitute). In certain embodiments, the cholesterol and cholesterol-substitute have a molar percentage ratio of less than about 100:0 (cholesterol:cholesterol-substitute).

In certain embodiments, the cholesterol and cholesterol-substitute have a molar percentage ratio of more than about 0:100 (cholesterol:cholesterol-substitute). In certain embodiments, the cholesterol and cholesterol-substitute have a molar percentage ratio of more than about 5:95 (cholesterol:cholesterol-substitute). In certain embodiments, the cholesterol and cholesterol-substitute have a molar percentage ratio of more than about 10:90 (cholesterol:cholesterol-substitute). In certain embodiments, the cholesterol and cholesterol-substitute have a molar percentage ratio of more than about 12.5:87.5 (cholesterol:cholesterol-substitute). In certain embodiments, the cholesterol and cholesterol-substitute have a molar percentage ratio of more than about 15:85 (cholesterol:cholesterol-substitute). In certain embodiments, the cholesterol and cholesterol-substitute have a molar percentage ratio of more than about 20:80 (cholesterol:cholesterol-substitute). In certain embodiments, the cholesterol and cholesterol-substitute have a molar percentage ratio of more than about 25:75 (cholesterol:cholesterol-substitute). In certain embodiments, the cholesterol and cholesterol-substitute have a molar percentage ratio of more than about 30:70 (cholesterol:cholesterol-substitute). In certain embodiments, the cholesterol and cholesterol-substitute have a molar percentage ratio of more than about 35:65 (cholesterol:cholesterol-substitute). In certain embodiments, the cholesterol and cholesterol-substitute have a molar percentage ratio of more than about 40:60 (cholesterol:cholesterol-substitute). In certain embodiments, the cholesterol and cholesterol-substitute have a molar percentage ratio of more than about 45:55 (cholesterol:cholesterol-substitute). In certain embodiments, the cholesterol and cholesterol-substitute have a molar percentage ratio of more than about 50:50 (cholesterol:cholesterol-substitute). In certain embodiments, the cholesterol and cholesterol-substitute have a molar percentage ratio of more than about 55:45 (cholesterol:cholesterol-substitute). In certain embodiments, the cholesterol and cholesterol-substitute have a molar percentage ratio of more than about 60:40 (cholesterol:cholesterol-substitute). In certain embodiments, the cholesterol and cholesterol-substitute have a molar percentage ratio of more than about 65:35 (cholesterol:cholesterol-substitute). In certain embodiments, the cholesterol and cholesterol-substitute have a molar percentage ratio of more than about 70:30 (cholesterol:cholesterol-substitute). In certain embodiments, the cholesterol and cholesterol-substitute have a molar percentage ratio of more than about 75:25 (cholesterol:cholesterol-substitute). In certain embodiments, the cholesterol and cholesterol-substitute have a molar percentage ratio of more than about 80:20 (cholesterol:cholesterol-substitute). In certain embodiments, the cholesterol and cholesterol-substitute have a molar percentage ratio of more than about 85:15 (cholesterol:cholesterol-substitute). In certain embodiments, the cholesterol and cholesterol-substitute have a molar percentage ratio of more than about 87.5:12.5 (cholesterol:cholesterol-substitute). In certain embodiments, the cholesterol and cholesterol-substitute have a molar percentage ratio of more than about 90:10 (cholesterol:cholesterol-substitute). In certain embodiments, the cholesterol and cholesterol-substitute have a molar percentage ratio of more than about 95:5 (cholesterol:cholesterol-substitute). In certain embodiments, the cholesterol and cholesterol-substitute have a molar percentage ratio of more than about 100:0 (cholesterol:cholesterol-substitute).

In certain embodiments, the cholesterol-substitute is chenodeoxycholic acid (CDCA). In certain embodiments, the cholesterol-substitute is cholic acid (CA). In certain embodiments, the cholesterol-substitute is deoxycholic acid (DCA). In certain embodiments, the cholesterol-substitute is lithocholic acid (LCA). In certain embodiments, the cholesterol-substitute is taurocholic acid. In certain embodiments, the cholesterol-substitute is glycocholic acid. In certain embodiments, the cholesterol-substitute is taurochenodeoxycholic acid. In certain embodiments, the cholesterol-substitute is glycochenodeoxycholic acid.

In certain embodiments, the ionizable lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the ionizable lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mol % of the LNP. In certain embodiments, the ionizable lipid comprises about 35 mol % of the LNP. In certain embodiments, the ionizable lipid comprises less than about 35 mol % of the LNP. In certain embodiments, the ionizable lipid comprises more than about 35 mol % of the LNP.

In certain embodiments, the ionizable lipid is C14-494.

In certain embodiments, the helper lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the helper lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the helper lipid comprises about 16 mol % of the LNP. In certain embodiments, the helper lipid comprises less than about 16 mol % of the LNP. In certain embodiments, the helper lipid comprises more than about 16 mol % of the LNP.

In certain embodiments, the helper lipid is dioleoyl-phosphatidylethanolamine (DOPE).

In certain embodiments, the PEG or PEG-conjugated lipid comprises about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 100 mol % of the LNP. In certain embodiments, the PEG or PEG-conjugated lipid comprises about 0.5, 0.6, 0.7, 0.8, 0.9, 1, 2, 3, 4, 5, 6, 7, 8, 9, 10, 11, 12, or about 13 mol % of the LNP. In certain embodiments, the PEG or PEG-conjugated lipid comprises about 2.5 mol % of the LNP. In certain embodiments, the PEG or PEG-conjugated lipid comprises less than about 2.5 mol % of the LNP. In certain embodiments, the PEG or PEG-conjugated lipid comprises more than about 2.5 mol % of the LNP.

In certain embodiments, the PEG or PEG-conjugated lipid comprises 1,2-dimyristoyl-sn-glycero-3-phosphoethanolamine-N-(methoxy(polyethyleneglycol)-2000) (C14PEG-2000).

In certain embodiments, the molar ratio of (a):(b):(c):(d) in the LNP is about 35:16:46.5:2.5.

### Lipids

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N-(N′,N′-dimethylaminoethane)-carbamoyl) cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In certain embodiments, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In certain embodiments, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

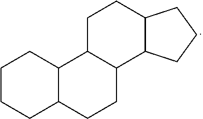 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In certain embodiments, the polyethylene glycol-lipid is N-[(methoxy poly(ethylene glycol)₂₀₀₀) carbamyl]-1,2-dimyristyloxlpropyl-3-amine (PEG-c-DMA). In certain embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl) butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl) carbamate or 2,3-di(tetradecanoxy)propyl-N-(ω-methoxy(polyethoxy)ethyl) carbamate.

In certain embodiments, the additional lipid is present in the LNP in an amount from about 1 mol % to about 10 mol %. In certain embodiments, the additional lipid is present in the LNP in an amount from about 1 mol % to about 5 mol %. In certain embodiments, the additional lipid is present in the LNP in about 1 mol % or about 2.5 mol %.

The term “lipid nanoparticle” refers to a particle having at least one dimension on the order of nanometers (e.g., 1-1,000 nm) which includes one or more lipids, for example a lipid of Formula (I)-(XV).

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In various embodiments, the lipids or the LNP of the present invention are substantially non-toxic.

In various embodiments, the lipids or the LNPs described herein are formulated for stability for in vivo immune cell targeting.

In some embodiments, the LNP formulated for stability for in vivo immune cell targeting comprises C14-4 in a concentration range of about 10 mol % to about 45 mol %. In some embodiments, the C14-4 is present in a molar ratio of about 40%.

In some embodiments, the LNP formulated for stability for in vivo immune cell targeting comprises a phospholipid in a concentration range of about 10 mol % to about 45 mol %. In certain embodiments, the phospholipid is dioleoyl-phosphatidylethanolamine (DOPE), and the DOPE is present in a molar ratio of about 25 or at a molar percentage of about 25%.

In some embodiments, the LNP formulated for stability for in vivo immune cell targeting comprises a cholesterol lipid in a concentration range of about 5 mol % to about 50 mol %. In certain embodiments, the cholesterol is present in a molar ratio of about 30, or at a molar percentage of about 30%.

In some embodiments, the LNP formulated for stability for in vivo immune cell targeting comprises total PEG in a concentration range of about 0.5 mol % to about 12.5 mol %. In certain embodiments, the total PEG is present in a molar ratio of about 2.5, or at a molar percentage of about 2.5%.

In certain embodiments, the LNP formulated for stability for in vivo immune cell targeting comprises ionizable lipid C14-4, DOPE, cholesterol and total PEG, wherein the C₁₄-4:DOPE:cholesterol: total PEG are present in a molar ratio of about 40:25:30:2.5 or at a molar percentage of about 40%: 25%: 30%: 2.5%.

In some embodiments, the total PEG comprises maleimide PEG (mPEG) and PEG in a mol ratio of about 1:1, 1:2, 1:3, 1:4, 1:5, 1:6, 1:7, 1:8, 1:9, 1:10, 1:11, 1:12, 1:13, 1:14, 1:15 or greater than 1:15, or any molar ratio therebetween. In certain embodiments, the LNP comprises total PEG at a mol ratio of about 2.5, wherein the total PEG comprises mPEG and PEG at a mol ratio of 1:3. In certain embodiments, the LNP comprises total PEG at a mol ratio of about 2.5, and the total PEG comprises PEG and mPEG at a mol ratio of 1:5. In certain embodiments, the LNP comprises total PEG at a mol ratio of about 2.5, and the total PEG comprises PEG and mPEG at a mol ratio of 1:7. In certain embodiments, the LNP comprises total PEG at a mol ratio of about 2.5, and the total PEG comprises PEG and mPEG at a mol ratio of 1:10.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In certain embodiments, the composition of the invention comprises an in vitro transcribed (IVT) RNA molecule. For example, in certain embodiments, the composition of the invention comprises an IVT RNA molecule which encodes an agent. In certain embodiments, the IVT RNA molecule of the present composition is a nucleoside-modified mRNA molecule. In certain embodiments, the agent is for targeting an immune cell to a pathogen or a tumor cell of interest. In certain embodiments, the IVT RNA molecule encodes a chimeric antigen receptor (CAR).

In some embodiments, the CAR is specific for binding to one or more antigens. In some embodiments, the antigen comprises at least one viral antigen, a bacterial antigen, a fungal antigen, a parasitic antigen, an influenza antigen, a tumor-associated antigen, a tumor-specific antigen, or any combination thereof.

However, the present invention is not limited to any particular agent or combination of agents. In certain embodiments, the composition comprises an adjuvant. In certain embodiments, the composition comprises a nucleic acid molecule encoding an adjuvant. In certain embodiments, the composition comprises a nucleoside-modified RNA encoding an adjuvant.

In certain embodiments, the composition comprises at least one RNA molecule encoding a combination of at least two agents. In certain embodiments, the composition comprises a combination of two or more RNA molecules encoding a combination of two or more agents.

In certain embodiments, the present invention provides a method for inducing an immune response in a subject. For example, the method can be used to provide immunity in the subject against a virus, bacteria, fungus, parasite, cancer, or the like. In some embodiments, the method comprises administering to the subject a composition comprising one or more LNP molecule formulated for in vivo targeting of an immune cell comprising one or more RNA encoding at least one antigen, an adjuvant, or a combination thereof.

In certain embodiments, the present invention provides a method for gene editing of an immune cell of a subject. For example, the method can be used to provide one or more component of a gene editing system (e.g., a component of a CRISPR system) to an immune cell of a subject. In some embodiments, the method comprises administering to the subject a composition comprising one or more ionizable LNP molecule formulated for targeted T cell delivery comprising one or more nucleoside-modified RNA molecule for gene editing.

In certain embodiments, the method comprises administration of the composition to a subject. In certain embodiments, the method comprises administering a plurality of doses to the subject. In some embodiments, the method comprises administering a single dose of the composition, where the single dose is effective in delivery of the target therapeutic agent.

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY(2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of PTPN22 using RNAi technology.

In one aspect, the invention includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote-and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl-methyl-, thio- and other modified forms of adenine, cytidine, guanine, thymine, and uridine.

In one embodiment of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or-independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3 ‘-terminus can be blocked with an aminoalkyl group. Other 3’ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the ICsQ. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Vaccine

In certain embodiments, the present invention provides an immunogenic composition for inducing or activating an immune response in a subject. For example, In certain embodiments, the immunogenic composition is a vaccine. As used herein, an “immunogenic composition” may comprise an LNP comprising an antigen (e.g., a peptide or polypeptide), an antibody or antibody fragment (e.g., an antigen binding molecule), a nucleic acid encoding an antigen or an antigen binding molecule, a cell expressing or presenting an antigen or an antigen binding molecule, or a combination thereof. In particular embodiments, the composition comprises or encodes all or part of any peptide antigen or antigen binding molecule, or an immunogenically functional equivalent thereof. In other embodiments, the composition comprises a mixture of mRNA molecules that encodes one or more additional immunostimulatory agent. Immunostimulatory agents include, but are not limited to, an additional antigen or antigen binding molecule, an immunomodulator, or an adjuvant. In the context of the present invention, the term “vaccine” refers to a substance that induces immunity upon inoculation into animals.

A vaccine of the present invention may vary in its composition of nucleic acid components. In a non-limiting example, a nucleic acid encoding an antigen or antigen binding molecule might also be formulated with an adjuvant. Of course, it will be understood that various compositions described herein may further comprise additional components. A vaccine of the present invention, and its various components, may be prepared and/or administered by any method disclosed herein or as would be known to one of ordinary skill in the art, in light of the present disclosure.

In some embodiments, the therapeutic compounds or compositions of the invention may be administered prophylactically (i.e., to prevent disease or disorder) or therapeutically (i.e., to treat disease or disorder) to subjects suffering from or at risk of (or susceptible to) developing the disease or disorder. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

### Nucleic Acids

In certain embodiments, the invention includes an ionizable LNP molecule formulated for targeted in vivo T cell delivery comprising or encapsulating one or more nucleic acid molecule. In certain embodiments, the nucleic acid molecule is a mRNA molecule. In certain embodiments, the mRNA molecule encodes a CAR. In certain embodiments, the nucleoside-modified mRNA molecule encodes a CAR. In certain embodiments, the invention includes a nucleoside-modified mRNA molecule encoding an adjuvant.

The nucleotide sequences encoding an CAR, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode an antigen or antigen binding molecule or adjuvant of interest.

Further, the scope of the invention includes nucleotide sequences that encode amino acid sequences that are substantially homologous to the amino acid sequences recited herein and preserve the immunogenic function of the original amino acid sequence.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215:403-410 (1990)).

In certain embodiments, the invention relates to a construct, comprising a nucleotide sequence encoding a CAR. In certain embodiments, the construct comprises a plurality of nucleotide sequences encoding a plurality of antigens. For example, in certain embodiments, the construct encodes 1 or more, 2 or more, 5 or more, 10 or more, 15 or more, or 20 or more antigens. In certain embodiments, the invention relates to a construct, comprising a nucleotide sequence encoding an adjuvant. In certain embodiments, the construct comprises a first nucleotide sequence encoding a CAR and a second nucleotide sequence encoding an adjuvant.

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding a CAR; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Vectors

The nucleic acid sequences encapsulated in the immune cell targeted LNP molecule of the invention can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid molecule of interest can be produced synthetically.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, sequencing vectors and vectors optimized for in vitro transcription.

In certain embodiments, the composition of the invention comprises in vitro transcribed (IVT) RNA encoding a CAR. In certain embodiments, the composition of the invention comprises IVT RNA encoding a plurality of antigens. In certain embodiments, the composition of the invention comprises IVT RNA encoding an adjuvant. In certain embodiments, the composition of the invention comprises IVT RNA encoding one or more antigens and one or more adjuvants.

### Nucleoside-modified RNA

In certain embodiments, the composition comprises a nucleoside-modified RNA. In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy. In certain embodiments, an immune cell comprising an expressing a mRNA molecule encoding the CAR is directed to a cell of interest expressing an antigen that is specifically bound by the CAR.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Kariko et al., 2011, Nucleic Acids Research 39: e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karikó et al., 2011, Nucleic Acids Research 39: e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In certain embodiments, the nucleoside-modified RNA of the invention is IVT RNA. For example, in certain embodiments, the nucleoside-modified RNA is synthesized by T7 phage RNA polymerase. In some embodiments, the nucleoside-modified mRNA is synthesized by SP6 phage RNA polymerase. In some embodiments, the nucleoside-modified RNA is synthesized by T3 phage RNA polymerase.

In certain embodiments, the modified nucleoside is m¹acp³Ψ (1-methyl-3-(3-amino-3-carboxypropyl) pseudouridine. In some embodiments, the modified nucleoside is m¹Ψ (1-methylpseudouridine). In some embodiments, the modified nucleoside is Ψm (2′-O-methylpseudouridine. In some embodiments, the modified nucleoside is m⁵D (5-methyldihydrouridine). In some embodiments, the modified nucleoside is m³Ψ (3-methylpseudouridine). In some embodiments, the modified nucleoside is a pseudouridine moiety that is not further modified. In some embodiments, the modified nucleoside is a monophosphate, diphosphate, or triphosphate of any of the above pseudouridines. In some embodiments, the modified nucleoside is any other pseudouridine-like nucleoside known in the art.

In some embodiments, the nucleoside that is modified in the nucleoside-modified RNA the present invention is uridine (U). In some embodiments, the modified nucleoside is cytidine (C). In some embodiments, the modified nucleoside is adenosine (A). In another embodiment the modified nucleoside is guanosine (G).

In some embodiments, the modified nucleoside of the present invention is m⁵C (5-methylcytidine). In some embodiments, the modified nucleoside is m⁵U (5-methyluridine). In some embodiments, the modified nucleoside is m⁶A (N⁶-methyladenosine). In some embodiments, the modified nucleoside is s²U (2-thiouridine). In some embodiments, the modified nucleoside is Ψ (pseudouridine). In some embodiments, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl) adenosine); ms²io6A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar (p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C(N⁴-acetylcytidine); f5° C. (5-formylcytidine); m⁵Cm (5,2′-O-dimethylcytidine); ac⁴Cm (N⁴-acetyl-2′-O-methylcytidine); k²C(lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho⁵U (5-hydroxyuridine); mo⁵U (5-methoxyuridine); cmo⁵U (uridine 5-oxyacetic acid); memo₅U (uridine 5-oxyacetic acid methyl ester); chm⁵U (5-(carboxyhydroxymethyl) uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In some embodiments, a nucleoside-modified RNA of the present invention comprises a combination of 2 or more of the above modifications. In some embodiments, the nucleoside-modified RNA comprises a combination of 3 or more of the above modifications. In some embodiments, the nucleoside-modified RNA comprises a combination of more than 3 of the above modifications.

In some embodiments, between 0.1% and 100% of the residues in the nucleoside-modified of the present invention are modified (e.g. either by the presence of pseudouridine or a modified nucleoside base). In some embodiments, 0.1% of the residues are modified. In some embodiments, the fraction of modified residues is 0.2%. In some embodiments, the fraction is 0.3%. In some embodiments, the fraction is 0.4%. In some embodiments, the fraction is 0.5%. In some embodiments, the fraction is 0.6%. In some embodiments, the fraction is 0.8%. In some embodiments, the fraction is 1%. In some embodiments, the fraction is 1.5%. In some embodiments, the fraction is 2%. In some embodiments, the fraction is 2.5%. In some embodiments, the fraction is 3%. In some embodiments, the fraction is 4%. In some embodiments, the fraction is 5%. In some embodiments, the fraction is 6%. In some embodiments, the fraction is 8%. In some embodiments, the fraction is 10%. In some embodiments, the fraction is 12%. In some embodiments, the fraction is 14%. In some embodiments, the fraction is 16%. In some embodiments, the fraction is 18%. In some embodiments, the fraction is 20%. In some embodiments, the fraction is 25%. In some embodiments, the fraction is 30%. In some embodiments, the fraction is 35%. In some embodiments, the fraction is 40%. In some embodiments, the fraction is 45%. In some embodiments, the fraction is 50%. In some embodiments, the fraction is 60%. In some embodiments, the fraction is 70%. In some embodiments, the fraction is 80%. In some embodiments, the fraction is 90%. In some embodiments, the fraction is 100%.

In some embodiments, the fraction is less than 5%. In some embodiments, the fraction is less than 3%. In some embodiments, the fraction is less than 1%. In some embodiments, the fraction is less than 2%. In some embodiments, the fraction is less than 4%. In some embodiments, the fraction is less than 6%. In some embodiments, the fraction is less than 8%. In some embodiments, the fraction is less than 10%. In some embodiments, the fraction is less than 12%. In some embodiments, the fraction is less than 15%. In some embodiments, the fraction is less than 20%. In some embodiments, the fraction is less than 30%. In some embodiments, the fraction is less than 40%. In some embodiments, the fraction is less than 50%. In some embodiments, the fraction is less than 60%. In some embodiments, the fraction is less than 70%.

In some embodiments, 0.1% of the residues of a given nucleoside (i.e., uridine, cytidine, guanosine, or adenosine) are modified. In some embodiments, the fraction of the given nucleotide that is modified is 0.2%. In some embodiments, the fraction is 0.3%. In some embodiments, the fraction is 0.4%. In some embodiments, the fraction is 0.5%. In some embodiments, the fraction is 0.6%. In some embodiments, the fraction is 0.8%. In some embodiments, the fraction is 1%. In some embodiments, the fraction is 1.5%. In some embodiments, the fraction is 2%. In some embodiments, the fraction is 2.5%. In some embodiments, the fraction is 3%. In some embodiments, the fraction is 4%. In some embodiments, the fraction is 5%. In some embodiments, the fraction is 6%. In some embodiments, the fraction is 8%. In some embodiments, the fraction is 10%. In some embodiments, the fraction is 12%. In some embodiments, the fraction is 14%. In some embodiments, the fraction is 16%. In some embodiments, the fraction is 18%. In some embodiments, the fraction is 20%. In some embodiments, the fraction is 25%. In some embodiments, the fraction is 30%. In some embodiments, the fraction is 35%. In some embodiments, the fraction is 40%. In some embodiments, the fraction is 45%. In some embodiments, the fraction is 50%. In some embodiments, the fraction is 60%. In some embodiments, the fraction is 70%. In some embodiments, the fraction is 80%. In some embodiments, the fraction is 90%. In some embodiments, the fraction is 100%.

In some embodiments, the fraction of the given nucleotide that is modified is less than 8%. In some embodiments, the fraction is less than 10%. In some embodiments, the fraction is less than 5%. In some embodiments, the fraction is less than 3%. In some embodiments, the fraction is less than 1%. In some embodiments, the fraction is less than 2%. In some embodiments, the fraction is less than 4%. In some embodiments, the fraction is less than 6%. In some embodiments, the fraction is less than 12%. In some embodiments, the fraction is less than 15%. In some embodiments, the fraction is less than 20%. In some embodiments, the fraction is less than 30%. In some embodiments, the fraction is less than 40%. In some embodiments, the fraction is less than 50%. In some embodiments, the fraction is less than 60%. In some embodiments, the fraction is less than 70%.

In some embodiments, a nucleoside-modified RNA of the present invention is translated in the cell more efficiently than an unmodified RNA molecule with the same sequence. In some embodiments, the nucleoside-modified RNA exhibits enhanced ability to be translated by a target cell. In some embodiments, translation is enhanced by a factor of 2-fold relative to its unmodified counterpart. In some embodiments, translation is enhanced by a 3-fold factor. In some embodiments, translation is enhanced by a 5-fold factor. In some embodiments, translation is enhanced by a 7-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the nucleoside-modified antigen-encoding RNA of the present invention induces significantly more adaptive immune response than an unmodified in vitro-synthesized RNA molecule with the same sequence. In some embodiments, the modified RNA molecule exhibits an adaptive immune response that is 2-fold greater than its unmodified counterpart. In some embodiments, the adaptive immune response is increased by a 3-fold factor. In another embodiment the adaptive immune response is increased by a 5-fold factor. In some embodiments, the adaptive immune response is increased by a 7-fold factor. In some embodiments, the adaptive immune response is increased by a 10-fold factor. In some embodiments, the adaptive immune response is increased by a 15-fold factor. In another embodiment the adaptive immune response is increased by a 20-fold factor. In some embodiments, the adaptive immune response is increased by a 50-fold factor. In some embodiments, the adaptive immune response is increased by a 100-fold factor. In some embodiments, the adaptive immune response is increased by a 200-fold factor. In some embodiments, the adaptive immune response is increased by a 500-fold factor. In some embodiments, the adaptive immune response is increased by a 1000-fold factor. In some embodiments, the adaptive immune response is increased by a 2000-fold factor. In some embodiments, the adaptive immune response is increased by another fold difference.

In some embodiments, “induces significantly more adaptive immune response” refers to a detectable increase in an adaptive immune response. In some embodiments, the term refers to a fold increase in the adaptive immune response (e.g., 1 of the fold increases enumerated above). In some embodiments, the term refers to an increase such that the nucleoside-modified RNA can be administered at a lower dose or frequency than an unmodified RNA molecule with the same species while still inducing an effective adaptive immune response. In some embodiments, the increase is such that the nucleoside-modified RNA can be administered using a single dose to induce an effective adaptive immune response.

In some embodiments, the nucleoside-modified RNA of the present invention exhibits significantly less innate immunogenicity than an unmodified in vitro-synthesized RNA molecule with the same sequence. In some embodiments, the modified RNA molecule exhibits an innate immune response that is 2-fold less than its unmodified counterpart. In some embodiments, innate immunogenicity is reduced by a 3-fold factor. In some embodiments, innate immunogenicity is reduced by a 5-fold factor. In some embodiments, innate immunogenicity is reduced by a 7-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor. In some embodiments, innate immunogenicity is reduced by another fold difference.

In some embodiments, “exhibits significantly less innate immunogenicity” refers to a detectable decrease in innate immunogenicity. In some embodiments, the term refers to a fold decrease in innate immunogenicity (e.g., 1 of the fold decreases enumerated above). In some embodiments, the term refers to a decrease such that an effective amount of the nucleoside-modified RNA can be administered without triggering a detectable innate immune response. In some embodiments, the term refers to a decrease such that the nucleoside-modified RNA can be repeatedly administered without eliciting an innate immune response sufficient to detectably reduce production of the recombinant protein. In some embodiments, the decrease is such that the nucleoside-modified RNA can be repeatedly administered without eliciting an innate immune response sufficient to eliminate detectable production of the recombinant protein.

### Polypeptide Therapeutic Agents

In other related aspects, the therapeutic agent includes an isolated peptide that modulates a target. For example, In certain embodiments, the peptide of the invention inhibits or activates a target directly by binding to the target thereby modulating the normal functional activity of the target. In certain embodiments, the peptide of the invention modulates the target by competing with endogenous proteins. In certain embodiments, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

CAR agents

In certain embodiments, the mRNA molecule of the invention encodes a chimeric antigen receptor (CAR). In certain embodiments, the CAR comprises an antigen binding domain. In certain embodiments, the antigen binding domain is a targeting domain, wherein the targeting domain directs the T cell expressing the CAR to a specific cell or tissue of interest. For example, In certain embodiments, the targeting domain comprises an antibody, antibody fragment, or peptide that specifically binds to an expressed on a pathogenic organism or a tumor cell thereby directing the T cell expressing the CAR to a cell or tissue expressing the antigen.

In certain embodiments, the invention relates to an immune cell targeted LNP comprising an agent, wherein the agent comprises a nucleic acid sequence encoding a chimeric antigen receptor (CAR). In certain embodiments, agent comprises an mRNA molecule encoding a CAR.

In certain embodiments, the agent comprises a modified nucleoside mRNA molecule encoding a CAR.

In various embodiments, the CAR can be a “first generation,” “second generation,” “third generation,” “fourth generation” or “fifth generation” CAR (see, for example, Sadelain et al., Cancer Discov. 3 (4): 388-398 (2013); Jensen et al., Immunol. Rev. 257:127-133 (2014); Sharpe et al., Dis. Model Mech. 8 (4): 337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD32-chain, which is the primary transmitter of signals from endogenous T cell receptors (TCRs). “First generation” CARs can provide de novo antigen recognition and cause activation of both CD4+ and CD8+ T cells through their CD3 chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3 signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1 (9): 1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD35 signaling domain in addition to a constitutive or inducible chemokine component.

“Fifth generation” CARs provide co-stimulation, for example, by CD28 or 4-1BB domains, and activation, for example, by a CD32 signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is an scFv specific for binding to a surface antigen of a target cell of interest (e.g., a pathogen or tumor cell.)

### Combinations

In certain embodiments, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Cell Targeting Domain

In various embodiments of the invention, the LNP of the invention is conjugated to a targeting domain specific for binding to a receptor of a target cell.

In certain embodiments, the target cell is a stem cell. Exemplary stem cells that can be targeted by the compositions of the invention include, but are not limited to, hematopoietic stem cells and stem cells related to hematopoietic stem cells (e.g., myeloid stem cells and lymphoid stem cells.)

In certain embodiments, the target cell is a peripheral blood mononuclear cell (PBMC).

In one cell the target cell is an immune cell. Exemplary immune cells that can be targeted according by the compositions of the invention include, but are not limited to, T cells, B cells, NK cells, antigen-presenting cells, dendritic cells, macrophages, monocytes, neutrophils, eosinophils, and basophils. In certain embodiments, the immune cell is a T cell. In some embodiments, T cells that can be targeted using the compositions of the invention can be CD4+ or CD8+ and can include, but are not limited to, T helper cells (CD4+), cytotoxic T cells (also referred to as cytotoxic T lymphocytes, CTL; CD8-T cells), and memory T cells, including central memory T cells (TCM), stem memory T cells (TSCM), stem-cell-like memory T cells (or stem-like memory T cells), and effector memory T cells, for example, T_{EM} cells and T_{EMRA} (CD45RA+) cells, effector T cells, Th1 cells, Th2 cells, Th9 cells, Th17 cells, Th22 cells, Tfh (follicular helper) cells, T regulatory cells, natural killer T cells, mucosal associated invariant T cells (MAIT), and γδ T cells. Major T cell subtypes include T_{N}(naive), T_{SCM} (stem cell memory), T_{CM} (central memory), T_{TM} (Transitional Memory), T_{EM} (Effector memory), and T_{TE} (Terminal Effector), TCR-transgenic T cells, T-cells redirected for universal cytokine-mediated killing (TRUCK), Tumor infiltrating T cells (TIL), CAR-T cells or any T cell that can be used for treating a disease or disorder.

In certain embodiments, the T cells of the invention are immunostimulatory cells, i.e., cells that mediate an immune response. Exemplary T cells that are immunostimulatory include, but are not limited to, T helper cells (CD4+), cytotoxic T cells (also referred to as cytotoxic T lymphocytes, CTL; CD8+ T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th17 cells, Th22 cells, Tfh (follicular helper) cells, natural killer T cells, mucosal associated invariant T cells (MAIT), and γδ T cells.

In certain embodiments, the T cell targeting domain binds to CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, or CCR7.

In certain embodiments, present invention relates to compositions comprising a combination of delivery vehicles conjugated to immune cell targeting domains for targeting multiple immune cells. In certain embodiments, the combination comprises two or more immune cell targeted delivery vehicles, targeting two or more immune cell antigens. In certain embodiments, the two or more immune cell antigens are selected from CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7. In certain embodiments, the combination comprises two or more T cell targeted delivery vehicles, targeting a surface antigen of a CD4+ T cell and a surface antigen of a CD8+ T cell. In certain embodiments, the combination comprises two or more T cell targeted delivery vehicles, targeting CD4 and CD8.

In certain embodiments, the targeting domain is conjugated to the LNP of the invention. Exemplary methods of conjugation can include, but are not limited to, covalent bonds, electrostatic interactions, and hydrophobic (“van der Waals”) interactions. In certain embodiments, the conjugation is a reversible conjugation, such that the delivery vehicle can be disassociated from the targeting domain upon exposure to certain conditions or chemical agents In some embodiments, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In certain embodiments, the activated polymer conjugated lipid comprises a first coupling group capable of reacting with a second coupling group. In certain embodiments, the activated polymer conjugated lipid is an activated pegylated lipid. In certain embodiments, the first coupling group is bound to the lipid portion of the pegylated lipid. In some embodiments, the first coupling group is bound to the polyethylene glycol portion of the pegylated lipid. In certain embodiments, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisiting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In certain embodiments, the first coupling group is a maleimide.

In certain embodiments, the second coupling group is a sulfhydryl group. The sulfhydryl group can be installed on the targeting domain using any method known to those of skill in the art. In certain embodiments, the sulfhydryl group is present on a free cysteine residue. In certain embodiments, the sulfhydryl group is revealed via reduction of a disulfide on the targeting domain, such as through reaction with 2-mercaptoethylamine. In certain embodiments, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone.

In certain embodiments, the targeting domain is conjugated to the LNP using maleimide conjugation.

### Targeting Domain

In certain embodiments, the composition comprises a targeting domain that directs the delivery vehicle to a target immune cell. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant invention include, but are not limited to, antibodies, antibody fragments, proteins, peptides, and nucleic acids.

In various embodiments, the targeting domain binds to a cell surface molecule of a cell of interest. For example, in various embodiments, the targeting domain binds to a cell surface molecule of an endothelial cell, a stem cell, or an immune cell.

### Peptides

In certain embodiments, the targeting domain of the invention comprises a peptide. In certain embodiments, the peptide targeting domain specifically binds to a target of interest.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215:403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Nucleic Acids

In certain embodiments, the targeting domain of the invention comprises an isolated nucleic acid, including for example a DNA oligonucleotide and a RNA oligonucleotide. In certain embodiments, the nucleic acid targeting domain specifically binds to a target of interest. For example, In certain embodiments, the nucleic acid comprises a nucleotide sequence that specifically binds to a target of interest.

The nucleotide sequences of a nucleic acid targeting domain can alternatively comprise sequence variations with respect to the original nucleotide sequences, for example, substitutions, insertions and/or deletions of one or more nucleotides, with the condition that the resulting nucleic acid functions as the original and specifically binds to the target of interest.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215:403-410 (1990)].

### Antibodies

In certain embodiments, the targeting domain of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Antigens

The present invention provides a composition that induces an immune response in a subject. In certain embodiments, the composition comprises an immune cell targeted LNP comprising a nucleic acid molecule encoding a chimeric antigen receptor CAR specific for an antigen.

In certain embodiments, the antigen comprises a polypeptide or peptide associated with a pathogen or tumor cell, such that the in vivo modified immune cell expressing the CAR is then targeted to the antigen, inducing an immune response against the antigen, and therefore the pathogen or tumor cell.

In certain embodiments, the antigen, recognized by the CAR encoded by the nucleic acid molecule, comprises a protein, peptide, a fragment thereof, or a variant thereof, or a combination thereof from any number of organisms, for example, a virus, a parasite, a bacterium, a fungus, or a mammal.

In certain embodiments, the antigen comprises a tumor-specific antigen or tumor-associated antigen, such that the immune cell expressing the CAR is directed to a tumor cell expressing the antigen.

### Viral Antigens

In certain embodiments, the antigen comprises a viral antigen, or fragment thereof, or variant thereof. In certain embodiments, the viral antigen is from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. In certain embodiments, the viral antigen is from papilloma viruses, for example, human papilloma virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, severe acute respiratory syndrome (SARS) virus, severe acute respiratory syndrome coronavirus 2 (SARS-COV-2) or a cancer causing virus.

### Parasite Antigens

In certain embodiments, the antigen comprises a parasite antigen or fragment or variant thereof. In certain embodiments, the parasite is a protozoa, helminth, or ectoparasite. In certain embodiments, the helminth (i.e., worm) is a flatworm (e.g., flukes and tapeworms), a thorny-headed worm, or a round worm (e.g., pinworms). In certain embodiments, the ectoparasite is lice, fleas, ticks, and mites.

In certain embodiments, the parasite is any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

In certain embodiments, the parasite is Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Bacterial Antigens

In certain embodiments, the antigen comprises a bacterial antigen or fragment or variant thereof. In certain embodiments, the bacterium is from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

In certain embodiments, the bacterium is a gram positive bacterium or a gram negative bacterium. In certain embodiments, the bacterium is an aerobic bacterium or an anaerobic bacterium. In certain embodiments, the bacterium is an autotrophic bacterium or a heterotrophic bacterium. In certain embodiments, the bacterium is a mesophile, a neutrophile, an extremophile, an acidophile, an alkaliphile, a thermophile, psychrophile, halophile, or an osmophile.

In certain embodiments, the bacterium is an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. In certain embodiments, bacterium is a mycobacteria, Clostridium tetani, Yersinia pestis, Bacillus anthracis, methicillin-resistant Staphylococcus aureus (MRSA), or Clostridium difficile.

### Fungal Antigens

In certain embodiments, the antigen comprises a fungal antigen or fragment or variant thereof. In certain embodiments, the fungus is Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Tumor Antigens

In certain embodiments, the antigen comprises a tumor antigen, including for example a tumor-associated antigen or a tumor-specific antigen. In the context of the present invention, “tumor antigen” or “hyperporoliferative disorder antigen” or “antigen associated with a hyperproliferative disorder” refer to antigens that are common to specific hyperproliferative disorders. In certain aspects, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

Tumor antigens are proteins that are produced by tumor cells that elicit an immune response, particularly T-cell mediated immune responses. In certain embodiments, the tumor antigen of the present invention comprises one or more antigenic cancer epitopes immunogenically recognized by tumor infiltrating lymphocytes (TIL) derived from a cancer tumor of a mammal. The selection of the antigen will depend on the particular type of cancer to be treated or prevented by way of the composition of the invention.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In certain embodiments, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG16, TA-90\Mac-2 binding protein\cyclophilin C-associated protein, TAAL6, TAG72, TLP, and TPS.

### Adjuvants

In certain embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Methods

In one aspect, the present disclosure provides a method of delivering at least one selected from the group consisting of a nucleic acid molecule and a therapeutic agent to a target cell. In certain embodiments, the method comprises administering to the subject a therapeutically effectively amount of at least one lipid nanoparticle (LNP), which is optionally formulated as a composition, such as but not limited to a pharmaceutical composition.

In certain embodiments, the LNP comprises at least one ionizable lipid, wherein the ionizable lipid comprises about 10 mol % to about 50 mol % of the LNP.

In certain embodiments, the LNP comprises at least one helper lipid, wherein the helper lipid comprises about 10 mol % to about 45 mol % of the LNP.

In certain embodiments, the LNP comprises at least one selected from the group consisting of cholesterol and a cholesterol-substitute, wherein the combination of the cholesterol and cholesterol-substitute comprise about 5 mol % to about 50 mol % of the LNP.

In certain embodiments, the LNP comprises at least one polyethylene glycol (PEG) or PEG-conjugated lipid, wherein the PEG or PEG conjugated lipid comprises about 0.5 mol % to about 12.5 mol % of the LNP.

In certain embodiments, the LNP comprises a cell targeting domain specific to binding to a surface molecule of a target cell. In certain embodiments, the cell targeting domain is covalently conjugated to at least one component of the LNP.

In certain embodiments, the ionizable lipid is an ionizable lipid of Formula (I), or a salt or solvate thereof:

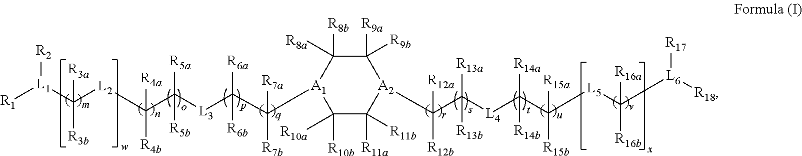

wherein:
A₁ and A₂ is independently selected from the group consisting of CH, N, and P;
L₁ and L₆ are each independently selected from the group consisting of CR₁₉ and N;
each occurrence of L₂ and L₅ is independently selected from the group consisting of —CH₂—, —CHR₁₉—, —O—, —NH—, and —NR₁₉—;
L₃ and L₄ are each independently selected from the group consisting of —CH₂—, —CHR₁₉—, —O—, —NH—, and —NR₁₉—;
each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆ₐ, R_{16b}, R₁₇, R₁₈, and R₁₉ is independently selected from the group consisting of H, halogen, optionally substituted C₁-C₂₈ alkyl, optionally substituted C₃-C₁₂ cycloalkyl, —Y(R₂₀)_{z′}(R₂₁)_{z″}-(optionally substituted C₃-C₁₂ cycloalkyl, optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₈ alkenyl, optionally substituted C₅-C₁₂ cycloalkenyl, —Y(R₂₀)_{z′}(R₂₁)_{z″}-(optionally substituted C₅-C₁₂ cycloalkenyl), optionally substituted C₂-C₂₈ alkynyl, optionally substituted C₆-C₁₂ cycloalkynyl, —Y(R₂₀)_{z′} (R₂₁)_{z″}-(optionally substituted C₆-C₁₂ cycloalkynyl), optionally substituted C₆-C₁₀ aryl, —Y(R₂₀)_{z′}(R₂₁)_{z″}-(optionally substituted C₆-C₁₀ aryl), optionally substituted C₂-C₁₂ heteroaryl, —Y(R₂₀)_{z′}(R₂₁)_{z″}-(optionally substituted C₂-C₁₂ heteroaryl), C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —Y(R₂₀)_{z′}(R₂₁)_{z″}-ester, —Y(R₂₀)_{z′} (R₂₁)_{z″}, —NO₂, —CN, and sulfoxy,
   or two geminal substituents selected from R₃ₐ and R_{3b}, R₄ₐ and R_{4b}, R₅ₐ and R_{5b}, R₆ₐ, and R_{6b}, R₇ₐ and R_{7b}, R₈ₐ and R_{8b}, R₉ₐ and R_{6b}, R₁₀ₐ and R_{10b}, R₁₁ₐ and R_{11b}, R₁₂ₐ and R_{12b}, R₁₃ₐ and R_{13b}, R₁₄ₐ and R_{14b}, or R₁₅ₐ and R_{15b} can combine with the C atom to which they are bound to form C═O;
each occurrence of Y is independently selected from the group consisting of C, N, O, S, and P;
each occurrence of R₂₀ and R₂₁ is independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —NO₂, —CN, and sulfoxy,
   or R₂₀ and R₂₁ can combine with the Y atom to which they are bound to form a C═O);
each occurrence of z′ and z″ is independently 0, 1, or 2; and
each occurrence of m, n, o, p, q, r, s, t, u, v, w, and x are is independently 0, 1, 2; 3, 4, or 5.

In certain embodiments, the ionizable lipid of Formula (I) is:

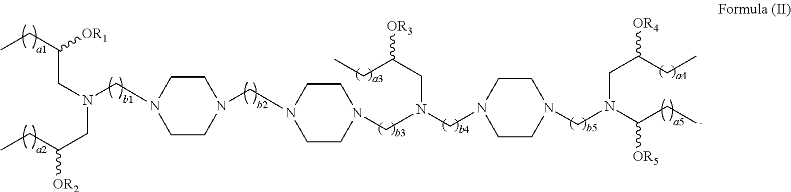

In certain embodiments, the ionizable lipid of Formula (I) is:

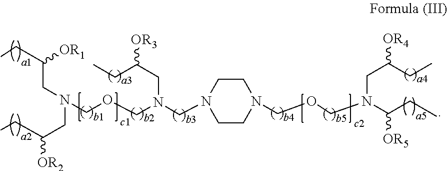

In certain embodiments, the ionizable lipid of Formula (I) is:

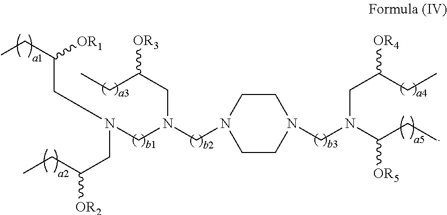

In certain embodiments, the ionizable lipid of Formula (I) is:

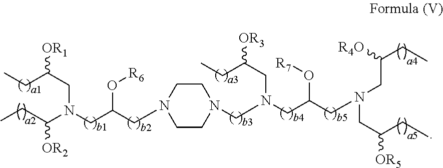

In certain embodiments, the ionizable lipid of Formula (I) is:

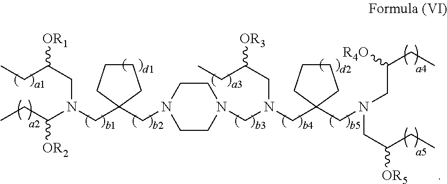

In certain embodiments, the ionizable lipid of Formula (I) is:

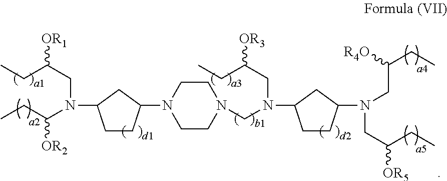

In certain embodiments, in the compounds of Formula (II), (III), (IV), (V), (VI), and (VII), the following definitions independently apply:
R₁, R₂, R₃, R₄, R₅, R₆, and R₇ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester;
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25;
b¹, b², b³, b⁴, and b⁵ are each independently 0, 1, 2, 3, 4, or 5;
c¹ and c² are each independently 0, 1, 2, 3, 4, or 5; and
d¹ and d² are each independently 0, 1, 2, 3, 4, or 5.

In certain embodiments, R₁, R₂, R₃, R₄, R₅, R₆, and R₇ are each independently selected from the group consisting of H, methyl, ethyl, iso-propyl, n-propyl, n-butyl, t-butyl, iso-butyl, and sec-butyl.

In certain embodiments, the ionizable lipid of Formula (I) is

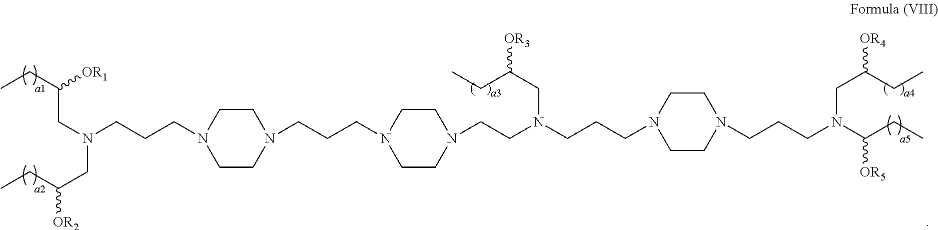

In certain embodiments, the ionizable lipid of Formula (I) is

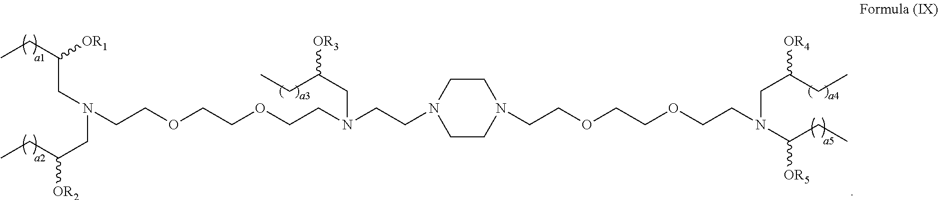

In certain embodiments, the ionizable lipid of Formula (I) is

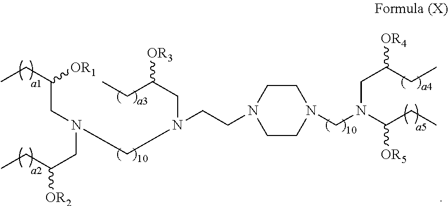

In certain embodiments, the ionizable lipid of Formula (I) is

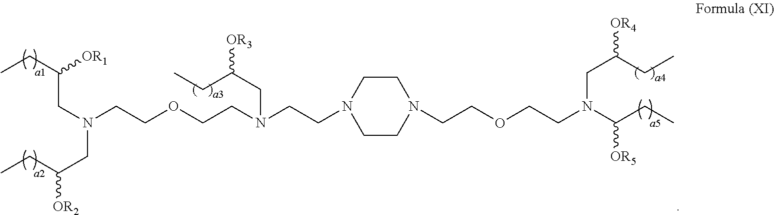

In certain embodiments, the ionizable lipid of Formula (I) is

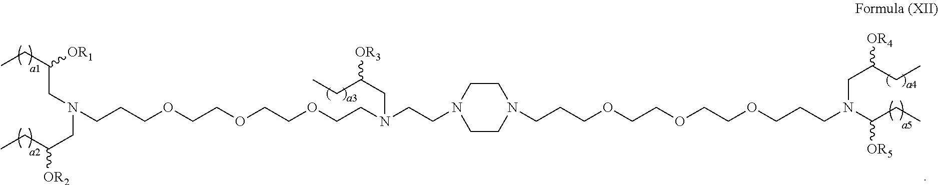

In certain embodiments, the ionizable lipid of Formula (I) is

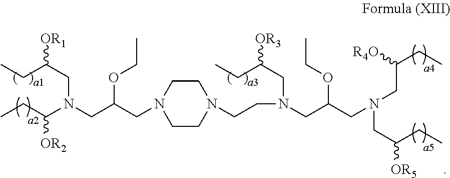

In certain embodiments, the ionizable lipid of Formula (I) is

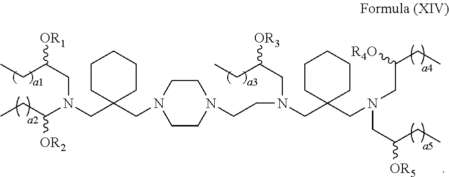

In certain embodiments, the ionizable lipid of Formula (I) is

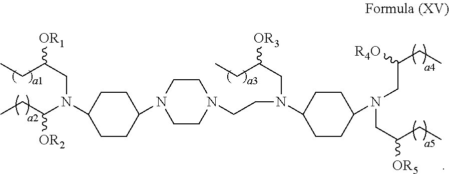

In certain embodiments, in the compounds of Formula (VIII), (IX), (X), (XI), (XII), (XIII), (XIV) and (XV), the following definitions independently apply:
R₁, R₂, R₃, R₄, and R₅ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester; and
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25.

In certain embodiments, R₁, R₂, R₃, R₄, and R₅ are each independently selected from the group consisting of H, methyl, ethyl, iso-propyl, n-propyl, n-butyl, t-butyl, iso-butyl, and sec-butyl.

In certain embodiments, the ionizable lipid of Formula (I) comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol):

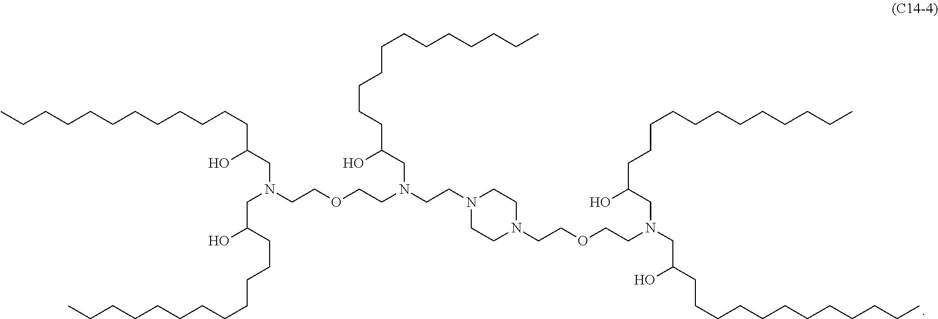 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

The present invention provides methods of delivering an agent to an immune cell of a target subject. In some embodiments, the agent is a diagnostic agent to detect at least one marker associated with a disease or disorder. In some embodiments, the agent is a therapeutic agent for the treatment or prevention of a disease or disorder. Therefore, in some embodiments, the invention provides methods for diagnosing, treating or preventing a disease or disorder comprising administering an effective amount of a composition comprising one or more diagnostic or therapeutic agents, one or more adjuvants, or a combination thereof.

In some embodiments, the method provides for delivery of compositions for gene editing or genetic manipulation to a target immune cell of a subject to treat or prevent a disease or disorder. Exemplary diseases or disorders include, but are not limited to, pathogenic disease and disorders and cancer.

In some embodiments, the method provides immunity in the target subject to an infection, or a disease, or disorder associated with an infectious agent. The present invention thus provides a method of treating or preventing the infection, or a disease, or disorder associated with an infectious agent. For example, the method may be used to treat or prevent a viral infection, bacterial infection, fungal infection, or a parasitic infection, depending upon the type of antigen of the administered composition. Exemplary antigens and associated infections, diseases, and tumors are described elsewhere herein.

The present invention also relates in part to methods of treating cancer and diseases or disorders associated therewith in subjects in need thereof, the method comprising the administration of a composition comprising at least one immune cell targeted LNP comprising a nucleic acid molecule encoding a CAR specific for binding to an tumor antigen for the treatment of cancer, or a disease or disorder associated therewith. Exemplary cancers that can be treated using the compositions and methods of the invention include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In certain embodiments, the composition is administered to a target subject having an infection, disease, or cancer. In certain embodiments, the composition is administered to a subject at risk for developing an infection, disease, or cancer. For example, the composition may be administered to a subject who is at risk for being in contact with a virus, bacteria, fungus, parasite, or the like.

In certain embodiments, the method comprises administering an immune cell targeted LNP comprising one or more nucleic acid molecules for treatment or prevention of a disease or disorder. In certain embodiments, the one or more nucleic acid molecules encode a therapeutic agent for the treatment of the disease or disorder. In certain embodiments, the one or more nucleic acid molecules encode an agent for targeting T cells to an antigen expressed by a pathogen or a cancer cell (e.g., an mRNA molecule encoding a chimeric antigen receptor).

In certain embodiments, the compositions of the invention can be administered in combination with an additional therapeutic agent, an adjuvant, or a combination thereof. For example, In certain embodiments, the method comprises administering a LNP comprising a nucleic acid molecule encoding one or more agent for targeting an immune cell to a pathogen or a tumor cell of interest and a second LNP comprising a nucleic acid molecule encoding one or more adjuvants. In certain embodiments, the method comprises administering a single LNP comprising a nucleic acid molecule encoding one or more agent for targeting an immune cell to a pathogen or a tumor cell of interest and a nucleic acid molecule encoding one or more adjuvants.

In certain embodiments, the method comprises administering to subject a plurality of nucleoside-modified nucleic acid molecules encoding a plurality of agents for targeting an immune cell to a pathogen or a tumor cell of interest, adjuvants, or a combination thereof.

In certain embodiments, the method of the invention allows for sustained expression of the agent for targeting an immune cell to a pathogen or a tumor cell of interest or adjuvant, described herein, for at least several days following administration. However, the method, in certain embodiments, also provides for transient expression, as in certain embodiments, the nucleic acid is not integrated into the subject genome.

In certain embodiments, the method comprises administering nucleoside-modified RNA which provides stable expression of the agent for targeting an immune cell to a pathogen or a tumor cell of interest or adjuvant described herein.

Administration of the compositions of the invention in a method of treatment can be achieved in a number of different ways, using methods known in the art. In certain embodiments, the method of the invention comprises systemic administration of the subject, including for example enteral or parenteral administration. In certain embodiments, the method comprises intradermal delivery of the composition. In some embodiments, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions encoding an agent for targeting an immune cell to a pathogen or a tumor cell of interest, adjuvant, or a combination thereof, described herein to practice the methods of the invention. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 10 nM and 10 μM in a mammal.

Typically, dosages which may be administered in a method of the invention to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 0.1 μg to about 10 mg per kilogram of body weight of the mammal. More preferably, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of an immunogenic composition or vaccine of the present invention may be performed by single administration or boosted by multiple administrations.

In certain embodiments, the invention includes a method comprising administering one or more compositions encoding one or more agent for targeting an immune cell to a pathogen or a tumor cell of interest or adjuvants described herein. In certain embodiments, the method has an additive effect, wherein the overall effect of the administering the combination is approximately equal to the sum of the effects of administering each agent for targeting an immune cell to a pathogen or a tumor cell of interest or adjuvant. In other embodiments, the method has a synergistic effect, wherein the overall effect of administering the combination is greater than the sum of the effects of administering each agent for targeting an immune cell to a pathogen or a tumor cell of interest or adjuvant.

### EXAMPLES

Various embodiments of the present application can be better understood by reference to the following Examples which are offered by way of illustration. The scope of the present application is not limited to the Examples given herein.

### Materials and Methods

### Materials

DLin-MC3-DMA (MC3) was purchased from MedChemExpress. Dexamethasone was obtained from Sigma-Aldrich (Saint Louis, MO). Other helper lipids were purchased from Avanti Polar Lipids (Alabaster, AL).

Production of the Luciferase mRNA

Codon optimized firefly luciferase was cloned into an mRNA production plasmid (optimized 3′ and 5′ UTR and containing a 101 polyA tail), in vitro transcribed in the presence in the presence of N¹-methylpseudouridine modified nucleoside (N¹mψ), co-transcriptionally capped using the CleanCap™ technology (TriLink) and cellulose purified to remove dsRNA. Purified mRNA was ethanol precipitated, washed, resuspended in nuclease-free water, and subjected to quality control (e.g., electrophoresis, dot blot, and transfection into human dendritic cells). mRNA was stored at −80° C. until use.

### Lipid Nanoparticle (LNP) Formulation and Characterization

In certain embodiments, an ethanol phase containing all lipids and an aqueous phase containing mRNA were mixed using a microfluidic device to synthesize LNPs. The ethanol phase comprised ionizable lipid (MC3), 1,2-distearyol-sn-glycero-3-phosphoethanolamine (DSPC), 1,2-dimyristoyl-sn-glycero-3-phosphoethanolamine-N-[methoxy(polyethyleneglycol)-(C14PEG-2000), cholesterol, and dexamethasone. MC3, DSPC, and C14PEG-2000 were combined at a molar ratio of 50%, 10% and 1.5% respectively. The molar ratios of cholesterol and dexamethasone vary by formulation and have a total molar ratio of 38.5%. The aqueous phase comprised luciferase mRNA dissolved in 10 mM citrate buffer. The ethanol and aqueous phases were mixed at a flow rate of 1.8 ml/min and 0.6 mL/min (3:1) respectively using Pump33DS syringe pumps (Harvard Apparatus, Holliston, MA). LNPs were placed in 1×PBS for dialysis in a microdialysis cassette (20,000 MWCO, Thermo Fisher Scientific, Waltham, MA) for 2 h and then filtered through a 0.22 μm filter. Zetasizer Nano (Malvern Instruments, Malvern, U.K.) was used to measure the polydispersity index (PDI) and Z-average diameters. mRNA concentration and encapsulation efficiency in each LNP formulation were measured by a modified Quant-iT RiboGreen (ThermoFisher) assay.

Lipid nanoparticles (LNPs) were alternatively synthesized through chaotic mixing between an ethanol phase and citric acid phase in a microfluidic device in a 1:3 volume ratio using pump33DS syringe pumps (Harvard Apparatus, Holliston, MA). The ethanol phase contained C14-4 ionizable lipid, 1,2-Dioleoyl-sn-glycero-3-phosphoethanolamine (DOPE) (Avanti Polar Lipids, Alabaster, AL), 1,2-dimyristoyl-sn-glycero-3-phosphoethanolamine-N-[methoxy(polyethylene glycol)-2000](PEG) (Avanti Polar Lipids), cholesterol (Avanti Polar Lipids), and X-hydroxycholesterol. The citric acid phase contained 10 mM citric acid and luciferase mRNA at 1 mg/mL. After synthesis, particles were subsequently dialyzed in 1× PBS for 2 hours and sterilely filtered through 0.22 μm filters.

### Library Design

In certain embodiments, library screening involved the evaluation of 6 hydroxycholesterols (i.e., cholesterols with hydroxyl groups added to various positions of cholesterol): 7α-hydroxycholesterol (Abcam, Cambridge, MA), 7β-hydroxycholesterol (Sigma Aldrich, St. Louis, MO), 19-hydroxycholesterol (Cayman Chemicals, Ann Arbor, MI), 20 (S)-hydroxycholesterol (Abcam, Cambridge, MA), 24 (S)-hydroxycholesterol (Cayman Chemicals, Ann Arbor, MI), 25-hydroxycholesterol (Abcam, Cambridge, MA). The library's base formulation excipient molar percentages were 35% C14-494, 16% DOPE, 46.5% Cholesterol, and 2.5% PEG. The six hydroxycholesterol candidates were incorporated into these formulations by substituting cholesterol with hydroxycholesterol at various molar substitution percentages (12.5%, 25%, 50%, 100%). The molar percentages of the excipients for these candidate formulations were maintained at 35% C14-494, 16% DOPE, 46.5% Total Cholesterol, and 2.5% PEG wherein total cholesterol constituted cholesterol and the hydroxycholesterol substitute.

### LNP Characterization

In certain embodiments, LNP sample mRNA concentration was determined using A₂₆₀ absorbance on the Infinite M Plex plate reader (Tecan, Morissville, NC). Z-average diameter (particle size) and polydispersity index (PDI) were determined using dynamic light scattering (DLS) on the Zetasizer Nano (Malvern Instruments, Malvern, UK). pKa was calculated by 6-(p-Toluidino)-2-naphthalenesulfonic Acid (TNS) assays. Buffered solutions of 150 mM sodium chloride, 20 mM sodium phosphate, 25 mM ammonium citrate, and 20 mM ammonium acetate were adjusted to reach pH values in increments of 0.5 from 2 to 12. LNPs were added to each pH-adjusted solution in a 96-well plate, and TNS was then added to each well for a final TNS concentration of 6 μM. The resulting fluorescence was read on the Infinite M Plex plate reader. The resulting data was fitted using a sigmoidal regression and pKa was calculated as the pH at which the fluorescence intensity reached 50% of its maximum value. Encapsulation efficiency was determined by the Quant-iT™ RiboGreen™ RNA Assay Kit (Thermo Fisher Scientific) as per manufacturer instructions.

### Cell Culture

Human hepatoma cell line HepG2 cells and murine macrophage cell line RAW264.7 cells were obtained from American Type Culture Collection (ATCC, Manassas, VA). They were cultured in Dulbecco's modified Eagle's medium (DMEM) supplemented with 10% fetal bovine serum (FBS) and 1% antibiotics (100 units/ml penicillin and 100 μg/ml streptomycin) and incubated at 37° C. in a humidified atmosphere with 5% CO₂.

Immortalized T cells, Jurkats, (ATCC no. TIB-152) were cultured in RPMI-1640 with L-glutamine (Thermo Fisher Scientific, Waltham, MA) supplemented with 10% fetal bovine serum (FBS) and 1% penicillin-streptomycin (P/S). Primary human T cells (CD3+) were collected from healthy volunteer donors and procured from the Human Immunology Core at Penn Medicine. Primary human T cells were subsequently combined in RPMI-1640 media with L-glutamine, 10% FBS, and 1% P/S at a 1:1 ratio of CD4+ to CD8+ T cells. Primary human T cells were subsequently activated with Human T-activator CD3/CD28 Dynabeads (Thermo Fisher Scientific) at a 1:1 bead to cell ratio. For all screening, cells were plated in 96-well plates at 60,000 cells per well in 60 μL of media. LNPs were then added to the wells at the desired mRNA dosage concentration (e.g., 60 ng per 60,000 cells). Cells were incubated for 24 hours before functional readout assays were performed.

### In Vitro Transfection and Cytotoxicity

HepG2 cells were seeded in a 96 well plate at a density of 1×10⁴ cells/well and were allowed to grow for 24 hours. LNPs with different cholesterol:dexamethasone (C:D) ratio (10:0, 9:1, 7:3, 5:5, 3:7, 0:10) were used to treat cells at a dose of 50 ng mRNA/well for 24 hours. Afterwards, luciferase expression and cell viability were tested using Luciferase Assay Kit (E4550, Promega) and CellTiter-Glo® Luminescent Cell Viability Assay Kit (G7572, Promega), respectively.

RAW264.7 cells were seeded in a 12-well plate at a density of 2×10⁵ cells/well and were allowed to grow for 24 hours. LNPs were used to treat cells at a dose of 500 ng mRNA/well for 24 hours. The supernatant was collected for TNF-α analysis.

### Animal Experiments

Nine 8- to 12-week-old C57BL/6 female mice (Jackson Laboratory, Bar Harbor, ME, ˜20 g) were divided randomly into three groups (n=3) and were intravenously injected with either PBS, the original MC3 LNP (C10D0), or the Dex-incorporated LNP (C9D1). For each mouse in the LNP-treated groups, 4 μg of luciferase mRNA was injected. 20 hours later, the blood was collected from each mouse through retro-orbital bleeding and the serum was prepared for TNF-α analysis. Bioluminescence imaging was performed with an IVIS Spectrum Imaging system (Caliper Life Sciences, Hopkinton, MA) 20 hours after the injection. D-luciferin (PerkinElmer, Waltham, MA) at a dose of 150 mg/kg was injected into mice by intraperitoneal (IP) injection, followed by anesthetization and imaging. The amount of total photon flux was measured.

### Cytokine Level Measurements

The concentration of TNF-α in RAW264.7 cultures and mouse serum were measured using a commercially available ELISA assay kit (Invitrogen).

### Statistical Analysis

Results were analyzed by an unpaired Student's t-test, and were expressed as mean values +/−SD or fold-increase using Prism 5 software package (Graphpad, Inc., San Diego, CA). Statistical significance was indicated by a p-value of equal to or less than 0.05.

### Luciferase and Toxicity Assays

For luciferase expression readout, 96-well plates were spun down at 300×g for 7 minutes. Supernatant media was removed, and cells were resuspended in 50 μL 1× lysis buffer (Promega, Madison, WI) and 100 μL luciferase assay substrate (Promega). Following 10 minutes of incubation, a plate reader was used to read luminescent signal from each well. Luminescence was normalized within each plate to S2. For toxicity assays, 60 μL of CellTiter-Glo™ (Promega) was added to each well. Following 10 minutes of incubation, a plate reader was used to read luminescent signal from each well. Luminescence was normalized within each plate to untreated cells.

Colocalization of Lipid Nanoparticles with Acidic Organelles

LNPs were diluted in 1× PBS to 10 ng luciferase mRNA/μL. Vybrant™ DiO Cell-Labeling Solution (Thermo Fisher Scientific) was added to LNP solutions at a 1:75 ratio by volume. Jurkat cells were treated at 60 ng luciferase mRNA/60,000 cells in 60 μL media for 3 hours. Cells were collected, centrifuged at 300×g for 5 minutes, resuspended in RPMI media containing LysoTracker™ Deep Red (Thermo Fisher Scientific) (1:3000), and incubated for an additional 1 hour. Cells were collected, centrifuged at 300×g for 5 minutes, and resuspended in 1×PBS. Cell-containing PBS solution was then allowed to sediment for 15 minutes onto a chambered microscope slide, Nunc™ Lab-Tek™ II 4-well Chamber Slide with removable wells (Thermo Fisher Scientific). PBS was aspirated off and slides were incubated with 4% formaldehyde for 10 minutes to fix cells. Slides were then washed 2 times with 1× PBS for 5 minutes each while rocking gently. Finally, chamber walls were removed, and cover slips were placed on the slides to prepare the samples for confocal microscopy imaging. Images were taken using Zeiss LSM 710 confocal microscope (Zeiss, Oberkochen, Germany).

### Endosomal Trafficking Assay

Jurkats were treated at either 60 of 150 ng luciferase mRNA/60,000 cells for 4 hours. At least 750,000 cells were used in each treatment group. Cells were collected, centrifuged at 300×g for 5 minutes, and resuspended in 1×PBS. Cell-containing PBS solution was then allowed to sediment for 15 minutes onto a chambered microscope slide, Nunc™ Lab-Tek™ II 4-well Chamber Slide with removable wells (Thermo Fisher Scientific). PBS was aspirated off and slides were incubated with 4% formaldehyde for 10 minutes to fix cells. All subsequent washes and incubations were done while gently rocking the slides. Slides were then washed with 1× PBS for 5 minutes and incubated with 0.3% Tween-20 in 1× PBS for 15 minutes. Next, slides were washed with 1×PBS, incubated/blocked with 2% bovine serum albumin (BSA) in 1× PBS for 30 minutes. Following the blocking step, slides were incubated for 1 hour with either Rab5A #46449, Rab7 #9367, or Rab11 #5589 XP® Rabbit mAb (Cell Signaling Technology, Danvers, MA) at 1:100. Following 2 washes in 1× PBS for 5 minutes each, cells were covered and incubated with Anti-rabbit IgG (H+L), F(ab′)2 Fragment (Alexa Fluor 647® Conjugate) #4144 (Cell Signaling Technology) at 1:1000 for 1 hour. Following 2 additional 1×PBS washes, slides were prepared using cover slips and ProLong™ Gold Antifade mountant (Thermo Fisher Scientific). Images were taken using Zeiss LSM 710 confocal microscope (Zeiss, Oberkochen, Germany).

### Imaging Software

For colocalization calculations, channels were overlayed with one another and coloc2, Fiji's built-in colocalization package. Spearman's rank correlation coefficient was recorded for a total of 5 image views (at least 75 total cells).

For total quantification of Rab5, Rab7, and Rab11 expression in endosomal trafficking assays, regions of interest were selected around cells using Fiji. Integrated density for each cell was then recorded (at least 50 total cells in each treatment group). Per cell Rab expression was then averaged and reported.

### Example 1: Rational Design of Exemplary Anti-Inflammatory Lipid Nanoparticles for mRNA Delivery

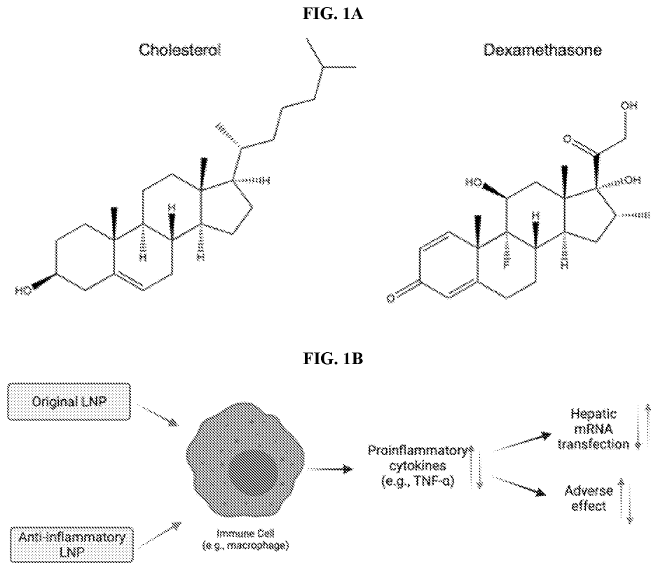

Corticosteroids possess anti-inflammatory effects and previous studies have shown that co-delivering genes and broad-spectrum anti-inflammatory steroids suppresses inflammation via inhibiting the transcription of proinflammatory genes. Dexamethasone (Dex) is a commonly used anti-inflammatory corticosteroid. Lipidated Dex was shown to reduce proinflammatory cytokines, suppress LNP-triggered immune activation, improve the tolerability of LNPs, and increase the expression of transgene. In addition, it has been recently demonstrated that DLin-MC3-DMA (MC3) LNPs co-delivering RNA therapeutics and anti-inflammatory steroids (e.g., rofleponide and budesonide) can suppress the inflammatory response and increase protein expression by 1.2-1.9 fold compared to the original formulation. Dex also shares structural similarities with cholesterol, one of the LNP components responsible for stabilizing LNP structure (FIG. 1A).

The experiments described herein demonstrate the development of an anti-inflammatory LNP formulation that co-delivers Dex and mRNA. The MC3 formulation that was approved by the FDA for siRNA delivery is explored in this study for further optimization, as previous research has shown that MC3 degrades slowly and is prone to trigger immune responses (Davies et al., 2021, Mol Ther Nucleic Acids, 24:369-384; Hou et al., 2021, Nat Rev Mater, 1-17; Hassett et al., 2019, Mol Ther Nucleic Acids, 15:1-11; Sabnis et al., 2018, Mol Ther, 26:1509-1519). By incorporating Dex directly into the LNP structure, the drug can be delivered to the same cells where LNPs can cause inflammatory responses and is therefore expected to suppress local inflammation caused by LNPs (FIG. 1B). From a translational point of view, the inclusion of an original form of Dex into LNPs should face less regulatory hurdles and scale-up problems than a Dex prodrug conjugated to LNPs, leading to the potential for broader applications of the new LNP formulation. The data presented herein demonstrate that Dex-incorporated LNPs effectively reduced the production of pro-inflammatory cytokine both in vitro and in vivo, and increased hepatic mRNA expression by 1.5-fold.

### Example 2: Characterization of Exemplary Anti-Inflammatory LNPs

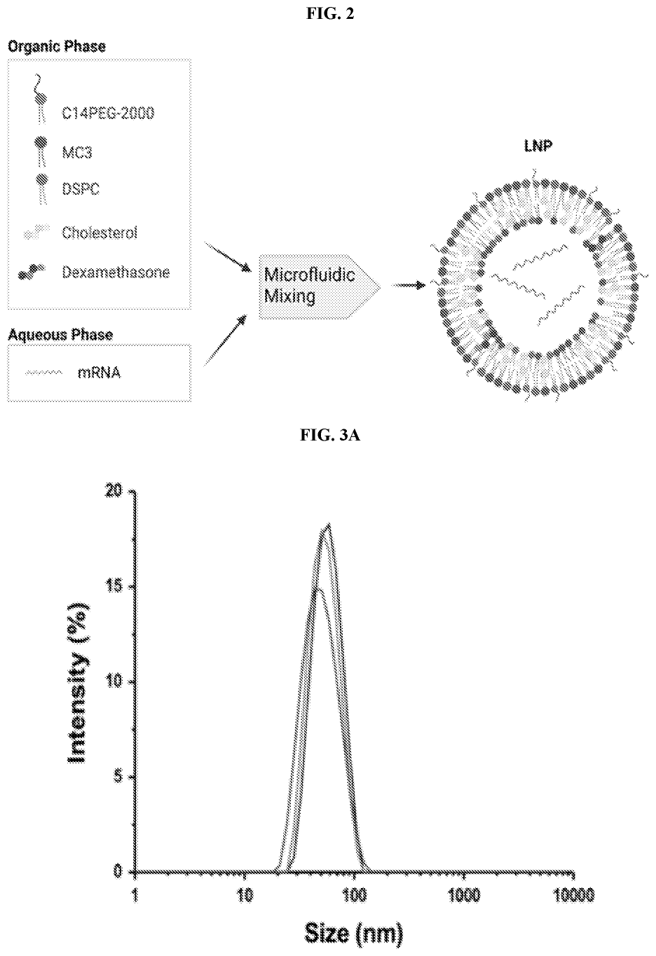

LNPs were formulated by mixing an aqueous phase containing mRNA and an organic phase comprising MC3, 1,2-distearyol-sn-glycero-3-phosphoethanolamine (DSPC), PEG conjugated lipid (C14PEG-2000), cholesterol, and dexamethasone in a microfluidic device (FIG. 2). The microfluidic device was designed to enable the formation of LNPs with a uniform size. To avoid immune activation by the mRNA, purified 1-methylpseudouridine-containing mRNA was utilized throughout the experiments described herein.

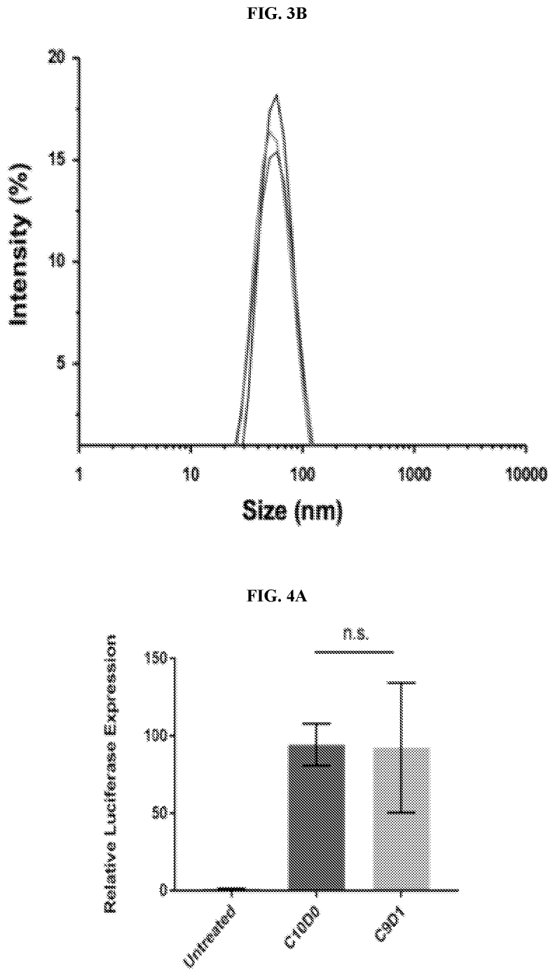

MC3 LNPs in the absence of Dex (C10D0) and Dex-incorporated LNPs (C9D1) were prepared. The naming convention follows the relative cholesterol:dexamethasone (C:D) ratio. That is, the formulation described as “C10D0” indicates that the relative C:D molar ratio is 10:0, and the C:D molar percentage in the C10D0 LNP is 38.5%: 0%. The C9D1 LNP has a C:D molar ratio of 9:1, and the C:D molar percentage is 34.65%: 3.8% (Table 1). Both LNPs had encapsulation efficiencies of >90% and were within the neutral range of ±10mV31. Moreover, hydrodynamic sizes as well as polydispersity were similar for both LNPs (FIGS. 3A-3B). These results suggest that the replacement of 10% cholesterol with Dex had minimal effect on size and polydispersity of LNPs, and that Dex-incorporated LNPs with high mRNA encapsulation efficiency were developed for subsequent studies.

TABLE 1
Characterization of Exemplary Anti-Inflammatory LNPs

| Property | C10D0 | C9D1 |
|---|---|---|
| C:D Ratio | 10:0 | 9:1 |
| C:D molar ratio in LNP (%) | 38.50:0 | 34.65:3.85 |
| Encapsulation Efficiency (%) | 92.52 | 93.46 |
| Z-diameter (nm) | 71.37 ± 0.017 | 76.84 ± 1.25 |
| Polydispersity Index (PDI) | 0.150 ± 0.017 | 0.124 ± 0.046 |
| Zeta-Potential | −5.27 ± 0.63 | −0.12 ± 0.10 |

C:D ratio represents the weight ratio between cholesterol and dexamethasone; ± represents SD.

### Example 3: In Vitro Transfection, Cytotoxicity, and Anti-Inflammatory Potential of C9D1 LNP

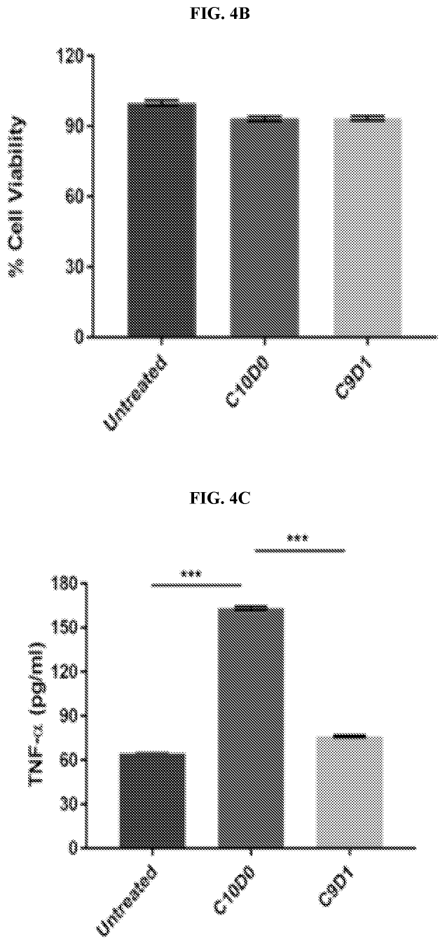

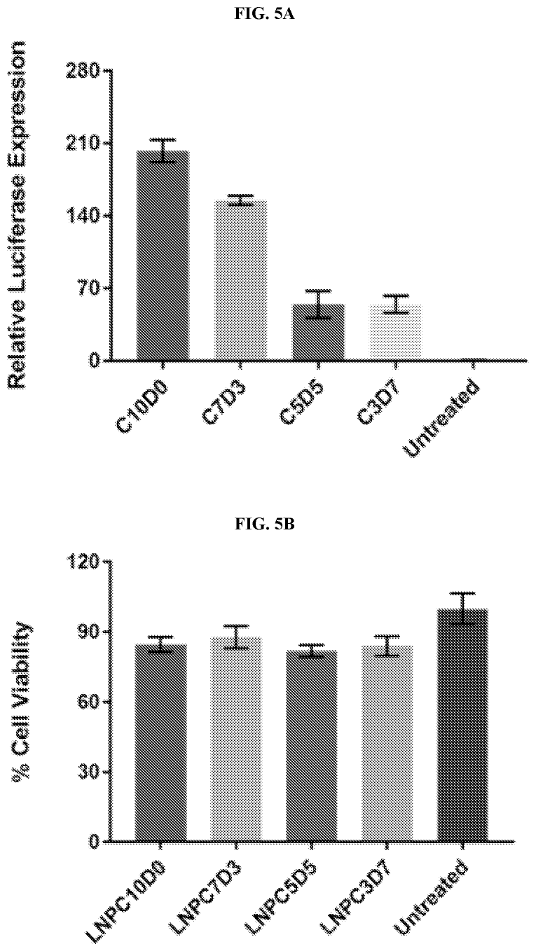

LNPs encapsulating mRNA encoding luciferase, in the presence or absence of Dex, were used to treat HepG2 cells to assess transfection efficiency and cytotoxicity. C9D1 LNPs did not show a reduction in transfection efficiency, as compared to C10D0 LNPs (FIG. 4A). Moreover, C9D1 LNP did not show increased cytotoxicity (FIG. 4B). Next, the possibility of incorporating additional Dex into LNPs was explored (Table 2). Although LNPs could still be formulated, the transfection efficiency dropped significantly as the proportion of Dex increased (FIGS. 5A-5B). These results suggest that the replacement of a fraction of cholesterol with Dex is key to maintaining high LNP transfection efficiency.

TABLE 2
Characterization of Exemplary Anti-Inflammatory LNPs

| Property | C10D0 | C7D3 | C5D5 | C3D7 | C0D10 |
|---|---|---|---|---|---|
| C:D Ratio | 10:0 | 7:3 | 5:5 | 3:7 | 0:10 |
| C:D molar | 38.50:0 | 26.95:11.55 | 19.25:19.25 | 11.55:26.95 | 0:38.50 |
| ratio in LNP | | | | | |
| (%) | | | | | |
| Encapsulation | 92.52 | 95.59 | 93.52 | 95.70 | 69.21 |
| Efficiency (%) | | | | | |
| Z-diameter | 71.37 ± 1.74 | 60.92 ± 2.89 | 81.13 ± 1.79 | 68.10 ± 1.82 | 76.69 ± 0.786 |
| (nm) | | | | | |
| Polydispersity | 0.150 ± 0.017 | 0.033 ± 0.024 | 0.130 ± 0.037 | 0.139 ± 0.039 | 0.040 ± 0.018 |
| Index (PDI) | | | | | |
| Zeta-Potential | −5.27 ± 0.63 | −4.07 ± 0.29 | −5.12 ± 0.52 | −4.00 ± 0.77 | −0.052 ± 0.132 |

C:D ratio represents the weight ratio between cholesterol and dexamethasone; ± represents SD.

To verify whether the incorporation of Dex can suppress the immune response triggered by the LNPs, the anti-inflammatory effect of C9D1 LNP on murine macrophages (RAW246.7) was evaluated (FIG. 4C). After the cells were stimulated with LNPs for 24 hours, the concentration of TNF-α in the supernatant was measured by enzyme linked immunosorbent assay (ELISA). While C10D0 LNP treatment significantly stimulated the production of TNF-α by approximately 2.6 fold in RAW246.7 cells, C9D1 LNP treatment only marginally increased TNF-α levels by 1.2 folds. These results indicate that C9D1 LNPs can suppress the immune response in vitro triggered by LNPs of the present disclosure.

### Example 4: In Vivo C9D1 LNP mRNA Delivery, Transfection, and Anti-Inflammatory Effect

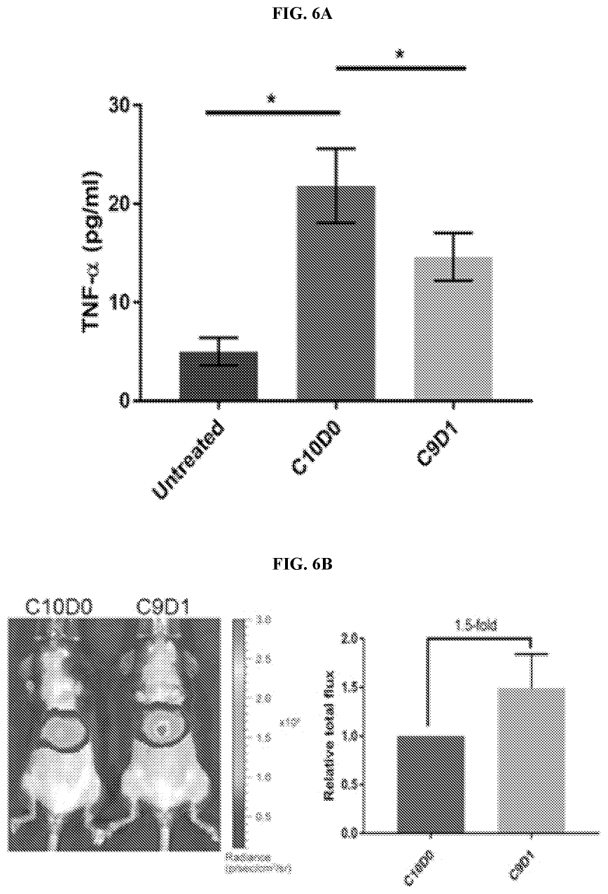

C57BL/6 mice were used to investigate the inflammatory response and mRNA delivery of LNPs of the present disclosure. LNPs comprising 4 μg of mRNA encoding luciferase were intravenously (i.v.) injected into each mouse. For the C9D1 LNP treatment group, the dose of Dex was 0.62 μg per mouse. Serum from untreated, C10D0 LNP, and C9D1 LNP groups were harvested for TNF-α quantification by ELISA. The results indicate that LNPs comprising unmodified cholesterol as the only cholesterol compound (i.e., no modified cholesterol and/or cholesterol analogs) induce an inflammatory response, as C10D0 LNP-treated mice showed a significantly higher TNF-α level than the untreated control group. In contrast, the serum TNF-α concentration of C9D1 LNP treated mice was significantly reduced compared to C10D0 LNP-treated mice (FIG. 6A). These results suggest that C9D1 LNP can successfully reduce the inflammatory response triggered by LNPs in vivo.

Previous studies used a higher dose of free Dex or Dex prodrug to suppress the inflammatory response induced by LNPs. However in the experiments described herein, without wishing to be bound by theory, the delivery of the lower-dose original Dex within the C9D1 LNP to the same cells where LNPs trigger inflammatory responses can be used to explain the successful suppression of inflammation. That is, an anti-inflammatory LNP that suppresses local immune responses could be a superior choice compared to suppressing systemic immune responses with a high dose of free corticosteroid.

Next, the in vivo transfection of C9D1 LNP encapsulated mRNA encoding luciferase was investigated. MC3 LNP is a clinically validated non-viral vector for liver transfection. Strong luciferase expression in the liver was observed for both C9D1 LNP- and C10D0 LNP-treated mice (FIG. 6B). Interestingly, quantification of the luminescence signal showed a 1.5-fold increase in C9D1 LNP-treated mice compared to C10D0 LNP-treated mice. This result is in line with previous reports that suggest that suppression of the immune response triggered by LNPs can increase gene expression. Since transgene expression can be suppressed in the presence of inflammatory cytokines such as TNF-α, C9D1 LNPs can enhance mRNA transfection by inhibiting the production of inflammatory cytokines. Taken together, the results indicate that C9D1 LNP is a promising formulation that can simultaneously reduce inflammation and enhance protein expression of mRNA/LNP therapeutics.

Dex-incorporated LNPs (C9D1) were successfully prepared and demonstrated potent anti-inflammatory effects. C9D1 LNPs were found to suppress the pro-inflammatory cytokine TNF-α to a near-basal level in vitro, and significantly down-regulated TNF-α levels in vivo compared to the native C10D0 LNP. Due to the reduced inflammatory responses, the overall mRNA transfection was improved by 1.5-fold in C9D1 LNP-treated mice. Therefore, LNPs comprising Dex represent promising strategy to reduce inflammation-related adverse effects of LNPs while enhancing protein expression of mRNA therapeutics.

### Example 5: Hydroxycholesterol Substitution in Ionizable Lipid Nanoparticles for mRNA Delivery to T Cells

The present disclosure relates, in part, to LNPs comprising a class of cholesterol analogs (i.e., hydroxycholesterols). In one aspect, the present disclosure describes the evaluation and/or impact of cholesterol analogs on LNP-mediated mRNA delivery to T cells. Hydroxycholesterols were selected as the excipient of interest given previous enzyme-ligand binding studies conducted on NPC1 and various cholesterol analogs. The addition of a hydroxyl group to various positions (e.g., polycyclic core and/or alkyl chain substituent of the 5-membered ring of the polycyclic core) along the cholesterol molecule can alter the binding kinetics between the modified cholesterol and NPC1. The goal of this alteration is to ultimately reduce NPC1 recognition of cholesterol during the endosomal trafficking of LNPs. However, cholesterol recognition by membrane proteins is still a critical step for LNP uptake. Therefore, the present disclosure describes evaluation of the substitution of six hydroxycholesterol candidates at four different substitution percentages to determine if any such substitutions improve delivery of mRNA to T cells.

### Example 6: Library Design for Exemplary LNPs Comprising Substituted Cholesterol

The present disclosure describes, in part, the design, synthesis, and evaluation of the delivery of mRNA to T cells with exemplary LNPs of the present disclosure. The base formulation of the library (i.e., S2) was a previously optimized formulation with the following excipients and molar ratio percentages: 35% C14-4 ionizable lipid, 46.5% cholesterol, 16% DOPE, and 2.5% lipid-anchored PEG. Notably, cholesterol makes up a significant molar percentage of the LNP formulation.

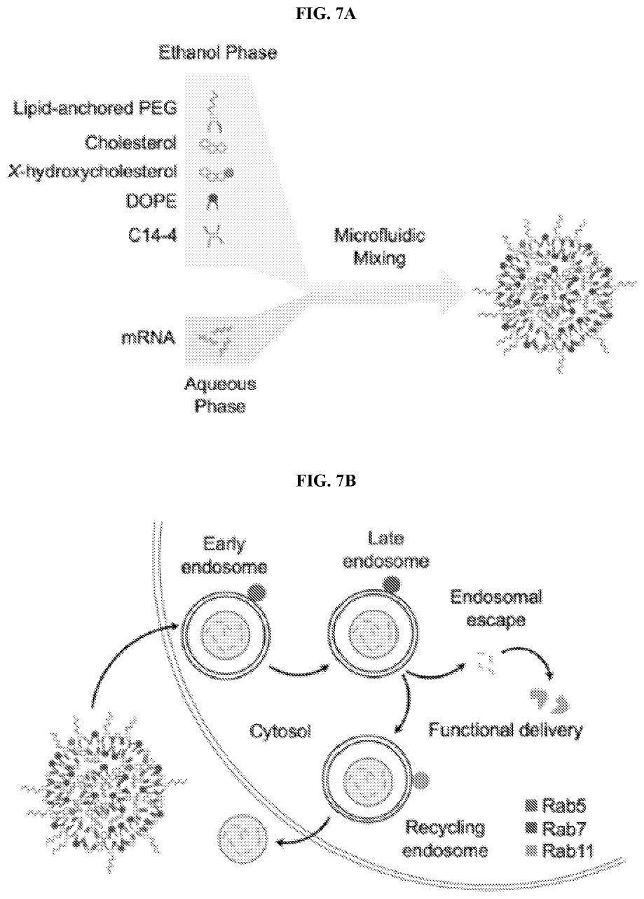

LNPs may be exocytosed from target cells through endosomal recycling. These pathways, specifically Niemann Pick type C1 (NPC1) mediated recycling, have been identified as core contributors to reduced functional delivery of nucleic acid cargos. Endosomal trafficking enzymes, such as NPC1, recognize lipids, especially cholesterol, and recycle these lipid components to the cell membrane. Enzyme-ligand binding studies have demonstrated that the addition of hydroxyl groups to the cholesterol molecule alter binding kinetics between NPC1 and the modified cholesterol. Endosomal trafficking was investigated to characterize the processing of LNPs through different stages of the endosome. The trafficking of endosomes through the cell can be tracked by the Ras-associated binding (Rab) family of proteins. Specifically, Rab5, Rab7, and Rab11 associate with the early, late, and recycling endosomes, respectively (FIG. 7B). Given that LNPs typically release mRNA cargo into the cytoplasm during the late endosome, it is apparent that LNPs that can reach the late endosome without being subsequently recycled have the greatest propensity for functional delivery.

The LNP library design described herein involved the substitution of a class of cholesterol analogs (i.e., hydroxycholesterols) into the S2 formulation at various substitution percentages (FIG. 7C). The motivation for hydroxycholesterol substitution into S2 was that such modifications of the cholesterol molecule may disrupt binding between NPC1 and cholesterol molecules, thereby reducing LNP recycling out of the cell.

Six hydroxycholesterol analogs were evaluated (i.e., 7α-hydroxycholesterol, 7β-hydroxycholesterol, 19-hydroxycholesterol, 20 (S)-hydroxycholesterol, 24 (S)-hydroxycholesterol, 25-hydroxycholesterol). These cholesterol substitutes were selected based on enzyme-ligand binding studies, location of hydroxyl group additions, and commercial availability. Many of these cholesterol analogs are found naturally in the body and result from the processing of cholesterol by reactive oxygen species and/or enzymes. For example, 7α-hydroxycholesterol is a bile acid precursor and 20 (S)-hydroxycholesterol participates in the Smoothened oncoprotein signaling pathway.

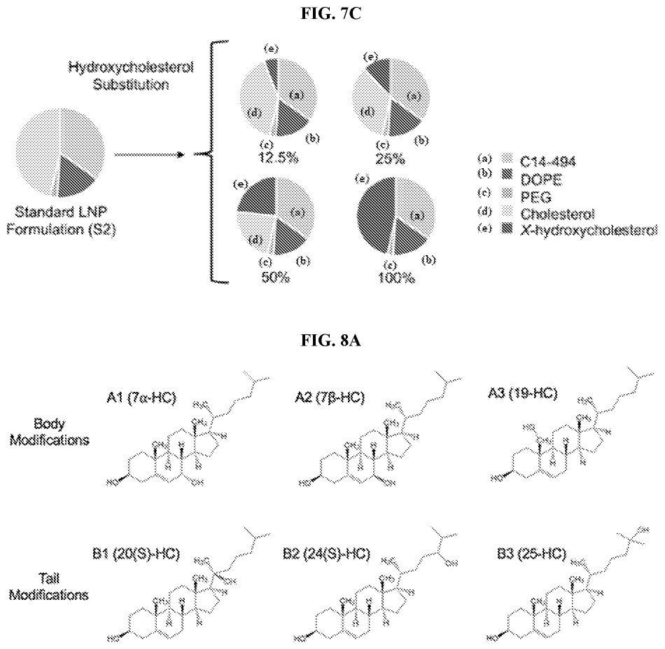

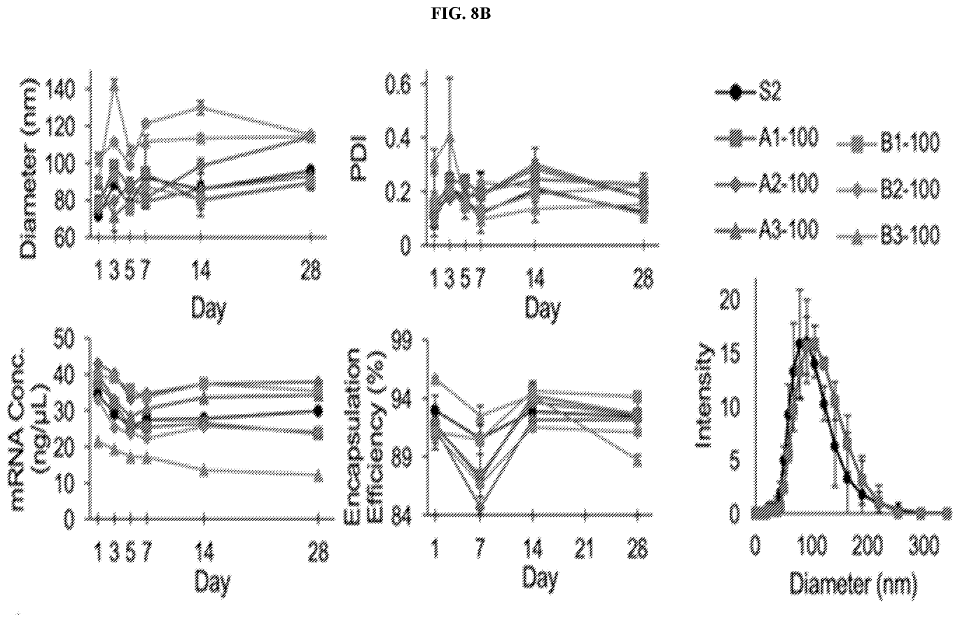

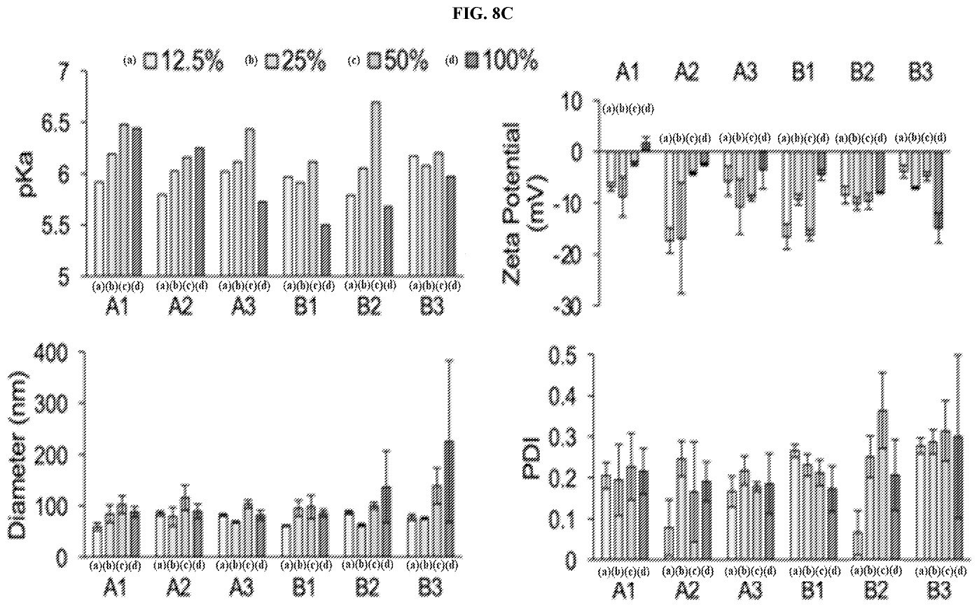

Each of the hydroxycholesterol substitutes is abbreviated A1, A2, A3, B1, B2, and B3, respectively. “A” substitutes (i.e., A1, A2, and A3) refer to analogs that have hydroxyl group additions to the ring structure (i.e., polycyclic core), or body, of the cholesterol molecule. “B” substitutes (i.e., B1, B2, and B3) refer to analogs that have hydroxyl group additions at the hydrophobic pole, or tail, of the cholesterol molecule (FIG. 8A). Each substitute was incorporated into the S2 formulation at either 12.5%, 25%, 50%, or 100% substitution percentages. This design scheme generated a total of 24 LNPs which are named by the hydroxycholesterol substitute and percentage substitution. For example, the A2-50 formulation is a 50% substitution of A2 into the S2 formulation.

An important consideration when introducing new excipients to LNP formulations is the effect of these additions on particle stability over time. This is largely because the chemical interactions between lipid components enable the formation of energetically stable membranes. In particular, the presence of cholesterol in lipid membranes impacts membrane stability and the intrinsic curvature of lipid bilayers by producing an ordering effect. The hydroxyl group at the head of the cholesterol molecule serves as a hydrophilic pole and causes cholesterol to be amphipathic. This enables cholesterol to orient itself along the normal of the lipid membrane and align neighboring lipids. Furthermore, the integration of the cholesterol molecule is energetically favorable due to nonpolar interactions between cholesterol and lipids.

Though the introduction of an additional hydroxyl group to the cholesterol molecule is intended to interfere with NPC1 binding, such modifications alter the nonpolar and electrostatic interactions that cholesterol has with other LNP excipients. Specifically, given the ordering effect that unmodified cholesterol has on membrane formation and stability, the addition of such a hydrophilic group to the body or tail of the cholesterol molecule could result in membrane instability. Furthermore, the addition of a hydroxyl group also may sterically hinder alignment of cholesterol and neighboring lipids. Therefore, it may be expected that the introduction of hydroxycholesterols into LNP formulations may reduce particle stability.

### Example 7: Stability of 100% Substitutions of Hydroxycholesterol Candidates into Standard LNP Formulation

Characterization parameters assessed over the 28-day period include z-average diameter, PDI, mRNA concentration, and encapsulation efficiency (Table 3). Trends indicate that 100% substitution of certain X-hydroxycholesterols for cholesterol in LNP formulations does not negatively impact stability. Regarding particle diameter and PDI, most of the 100% substitution LNPs maintained sizes between 60 and 100 nm and PDIs below 0.25 over the 28-day period. Temporal trends in mRNA concentration and encapsulation efficiency over time were also similar between all LNP candidates and S2 (FIG. 8B).

TABLE 3
LNP library characterization data for selected
exemplary cholesterol-substituted LNPs

| | Encapsulation | Z-Average Size | Polydispersity | Zeta Potential | |
|---|---|---|---|---|---|
| Particle | Efficiency (%) | (nm) | Index (PDI) | (mV) | pKa |
| S2 | 91 | 90.6 ± 29.31 | 0.19 ± 0.07 | −4.36 ± 3.73 | 5.9 |
| A1-12.5 | 96 | 59.35 ± 7.43 | 0.2 ± 0.03 | −6.9 ± 0.8 | 5.92 |
| A2-12.5 | 96 | 84.88 ± 4.61 | 0.08 ± 0.07 | −17.33 ± 2.46 | 5.79 |
| A3-12.5 | 96 | 82.05 ± 2.34 | 0.17 ± 0.04 | −5.75 ± 2.88 | 6.02 |
| B1-12.5 | 95 | 61.03 ± 1.18 | 0.26 ± 0.02 | −16.57 ± 2.48 | 5.97 |
| B2-12.5 | 95 | 86.95 ± 3.91 | 0.07 ± 0.05 | −8.48 ± 1.67 | 5.79 |
| B3-12.5 | 95 | 77.52 ± 5.98 | 0.28 ± 0.02 | −3.84 ± 1.21 | 6.17 |
| A1-25 | 90 | 84.44 ± 16.88 | 0.19 ± 0.09 | −8.82 ± 3.86 | 6.19 |
| A2-25 | 91 | 78.52 ± 18.28 | 0.25 ± 0.04 | −16.89 ± 10.8 | 6.03 |
| A3-25 | 94 | 68.52 ± 1.79 | 0.22 ± 0.04 | −10.75 ± 5.35 | 6.12 |
| B1-25 | 95 | 95.1 ± 15.12 | 0.23 ± 0.03 | −9.33 ± 1.07 | 5.91 |
| B2-25 | 95 | 62.44 ± 2.66 | 0.25 ± 0.05 | −10.18 ± 1.24 | 6.05 |
| B3-25 | 94 | 75.39 ± 2.09 | 0.29 ± 0.03 | −7.07 ± 0.25 | 6.08 |
| A1-50 | 95 | 102.15 ± 16.97 | 0.23 ± 0.08 | −2.36 ± 0.43 | 6.48 |
| A2-50 | 95 | 115.68 ± 24.44 | 0.17 ± 0.12 | −4.2 ± 0.36 | 6.16 |
| A3-50 | 95 | 103.12 ± 7.73 | 0.18 ± 0.01 | −9 ± 0.63 | 6.43 |
| B1-50 | 96 | 97.6 ± 22.94 | 0.21 ± 0.03 | −16.23 ± 1.08 | 6.12 |
| B2-50 | 95 | 100.07 ± 6.55 | 0.36 ± 0.09 | −9.68 ± 1.55 | 6.7 |
| B3-50 | 95 | 138.7 ± 35.24 | 0.31 ± 0.07 | −4.83 ± 0.88 | 6.2 |
| A1-100 | 92 | 88.08 ± 10.05 | 0.22 ± 0.06 | 1.67 ± 1.13 | 6.44 |
| A2-100 | 88 | 89.45 ± 13.88 | 0.19 ± 0.05 | −2.46 ± 0.27 | 6.25 |
| A3-100 | 91 | 81.79 ± 9.16 | 0.19 ± 0.07 | −3.58 ± 3.7 | 5.73 |
| B1-100 | 92 | 85.72 ± 6.89 | 0.17 ± 0.06 | −4.55 ± 1.04 | 5.5 |
| B2-100 | 92 | 137.02 ± 69.77 | 0.21 ± 0.09 | −8.02 ± 0.16 | 5.68 |
| B3-100 | 88 | 225.48 ± 157.36 | 0.3 ± 0.2 | −14.9 ± 2.9 | 5.97 |

Notably, however, B2-100 and B3-100 exhibited some instable characteristics. B2-100 and B3-100 both had average diameters above 100 nm and trended lower in sample mRNA concentration. Furthermore, B3-100 had significant variation in PDI over the 28-day period. B2-100 and B3-100 represent 100% substitutions of 24 (S)-hydroxycholesterol and 25-hydroxycholesterol for cholesterol, respectively, which are tail modifications of the cholesterol molecule. It has been proposed that the addition of a hydroxyl group at the tail end (i.e., alkyl substituent of the 5-membered ring of the polycyclic core of cholesterol) of the cholesterol molecule altered its amphipathic nature, potentially preventing its alignment within the lipid membrane and resulting to this observed instability. The remaining four hydroxycholesterol candidates (i.e., A1, A2, A3, and B1), in contrast, were comparable on all parameters on all days to S2.

Given that 100% substitution formulations varied in stability over time, formulations that had 12.5%, 25%, and 50% substitutions of their respective X-hydroxycholesterol candidate for cholesterol were characterized with respect to at least pKa, zeta potential, z-average diameter, and PDI (FIG. 8C). LNP pKa provides data indicating the endocytic pH at which the particle will release cargo into the cytoplasm. Trends indicate that for LNPs involving substitutions of body-modified hydroxycholesterols, increasing substitution percentage is associated with increasing pKa, suggesting that such substitutions may play a role in endosomal escape. Zeta potential measurements did not trend in a particular direction. While A1, A2, A3 (i.e., body modifications), and B1 remained within expected ranges for diameter and PDI, B2 and B3 had increased particle diameters and were more polydisperse. This further supports the previous finding that tail modifications at the 24 or 25 terminus of the cholesterol molecule disrupts normal synthesis and LNP formation, possibly by reducing cholesterol's ordering effect.

### Example 8: In Vitro Screen of Exemplary LNPs of the Present Disclosure

Though particle stability is an important consideration for LNP design and evaluation, functional delivery is also an important metric. Cholesterol is critical to membrane fusion, thus removing it completely from LNP formulations may result in reduced endosomal uptake. Therefore, a library of 24 LNPs comprising 6 hydroxycholesterol substitutes at 4 different substitution percentages, were evaluated in immortalized T cells, Jurkats, using luciferase expression and cell viability as primary readouts.

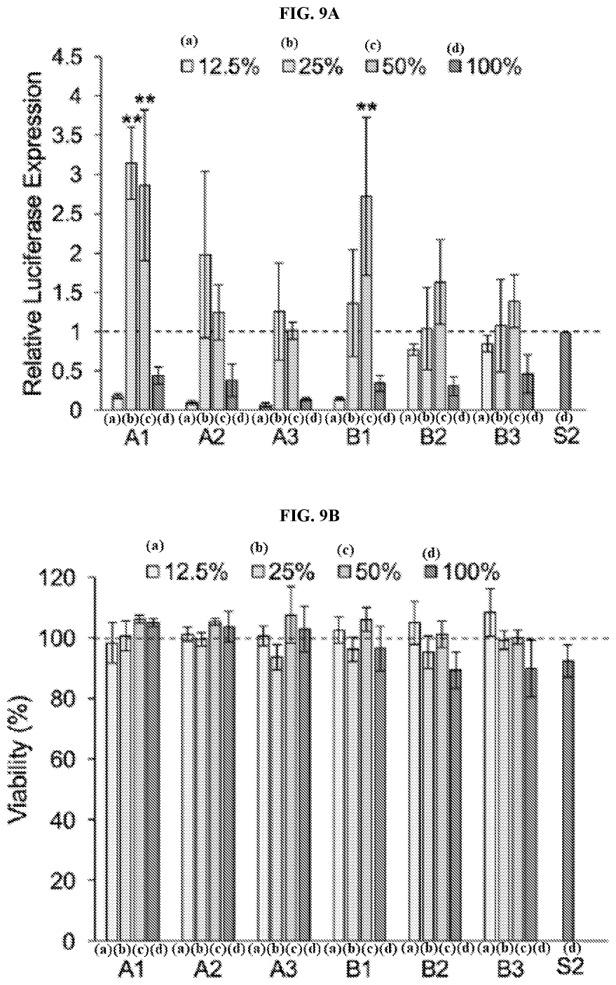

This screen revealed that within each hydroxycholesterol substitute category, relative luciferase expression as compared with S2 tended to be unimodal with moderate-performing LNPs having low and high substitution percentages (i.e., 12.5% and 100%) and high-performing LNPs having moderate substitution percentages (i.e., 25% and 50%) (FIGS. 9A-9B). The screen further revealed that A1-25, A1-50, and B1-50 produced statistically significant improvements in mRNA delivery to Jurkats as compared to S2 of 214%, 186%, and 172%, respectively. Furthermore, none of the LNP formulations in the library produced significant changes in cell viability, suggesting that the incorporation of hydroxycholesterol substitutes into LNPs does not induce increased cell death. Ultimately, A1-25, A1-50, and B1-50 demonstrated increased delivery of mRNA cargo with no significant change to particle toxicity in vitro.

### Example 9: Ex Vivo Screen of LNPs Comprising Hydroxycholesterol Candidates in Primary Human T Cells

To further explore the translatability of these modified lipid nanoparticles to ex vivo applications, a secondary screen of 12 LNPs was conducted with LNPS comprising the A1, A2, and B1 substitutes, in primary human T cells. A1 and B1 were selected because A1-25, A1-50, and B1-50 performed better than S2 in an in vitro evaluation. Although none of the A2 candidates significantly improved delivery of mRNA in vitro, A2-containing LNPs were also included in this ex vivo evaluation because of the similarity of A1 and A2 as stereoisomers. In order to deliver mRNA to primary T cells, the T cells must be activated with CD3/CD28 dependent pathways. As such, these expansion triggers may alter the membrane homeostasis of the cells. As such, all substitution percentages for the 3 selected hydroxycholesterol substitutes (i.e., A1, A2, B2) were evaluated in this ex vivo assay to re-optimize substitution percentages for ex vivo applications.

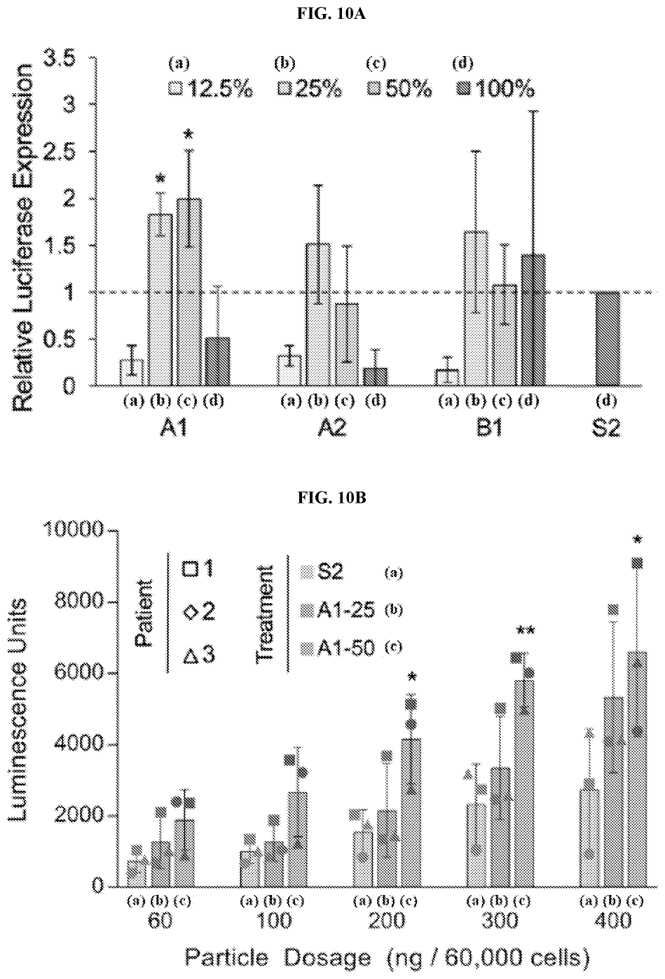

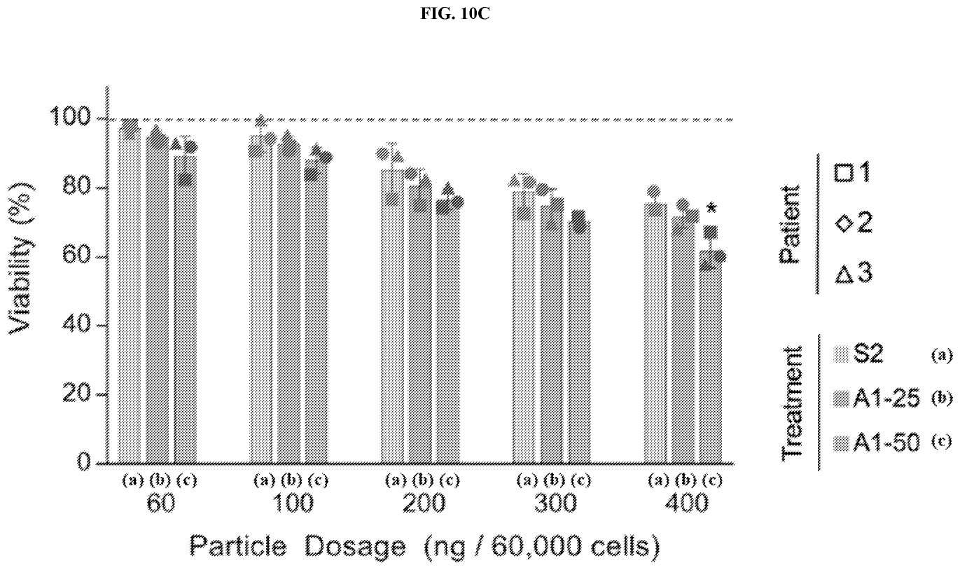

Despite patient-to-patient variability, the screen revealed that A1-25 and A1-50 significantly improve mRNA delivery by 83% and 99%, respectively, to primary T cells as compared to S2 (FIGS. 10A-10C). Interestingly, A1-25 outperformed A1-50 in vitro while A1-50 outperformed A1-25 ex vivo. Without wishing to be bound by theory, this result may be due to the inherent differences in endocytic activity exhibited by activated primary T cells and immortalized T cells.

To further validate these findings, a dose response assay in primary human T cells was conducted and revealed that A1-25 and A1-50 sustain improvements in mRNA delivery to T cells at dosages ranging from 60 to 400 nanograms of mRNA per 60,000 cells with little to no significant increase in cell viability. This suggests that A1-25 and A1-50 can be utilized in ex vivo applications, such as CAR T cell therapy, to increase mRNA delivery efficiency without increasing toxicity towards target cells.

### Example 10: Colocalization of Top LNP Formulations with Endosomes in Jurkats

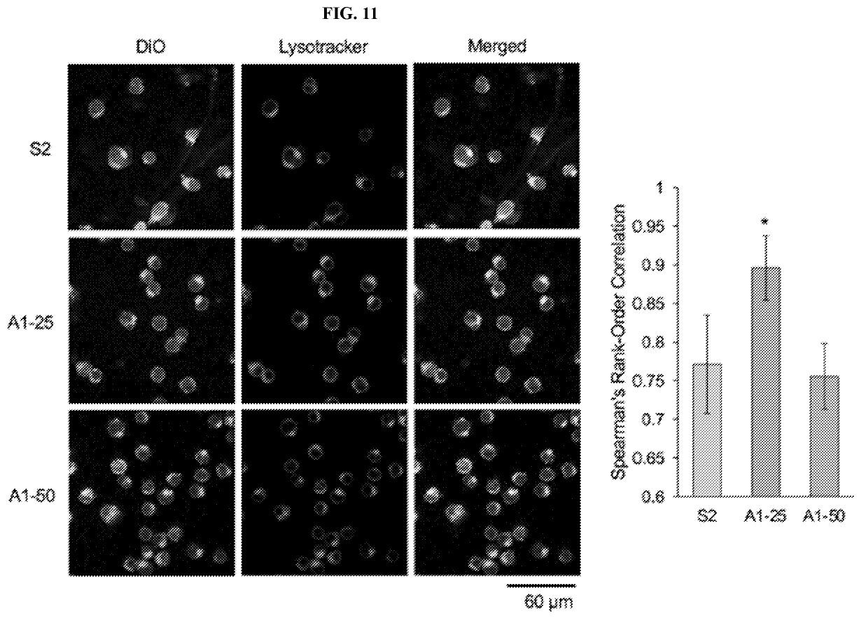

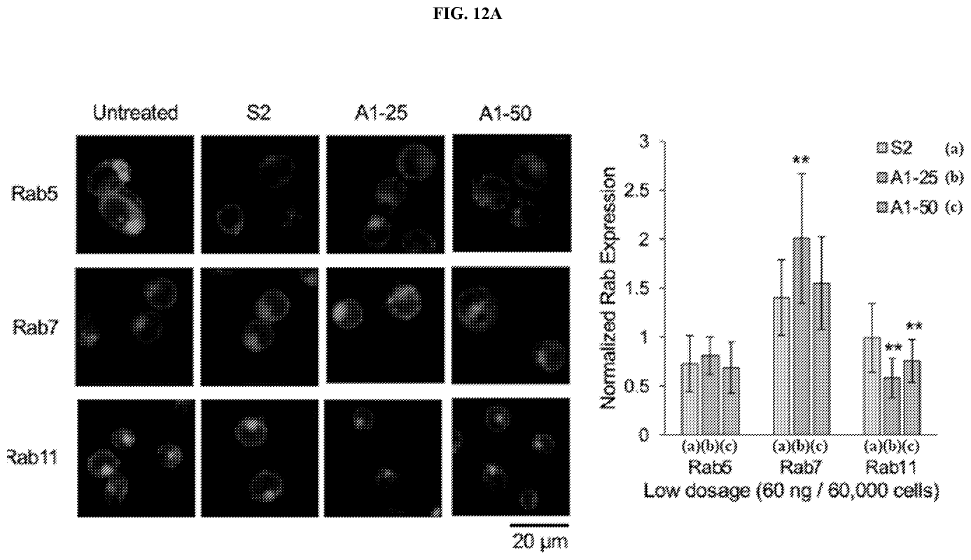

To better understand the impact of these hydroxycholesterol substitutions on endosomal uptake and retention, a colocalization assay was utilized to assess the accumulation of LNPs within acidic organelles in Jurkat cells. Lysotracker was used to mark spherical, acidic organelles, the majority of which are endosomes and lysosomes, while LNPs were labeled with DiO, a lipophilic dye. A1-25 demonstrated increased colocalization with these acidic organelles, suggesting that the A1-25 particle either enters cells at higher rates or remains in endosomes for longer periods of time (FIG. 11). It has previously been observed that for LNPs to release cargo and enable mRNA transcription, LNPs must reach and remain in the late endosome. As such, either increases in particle uptake or a greater frequency of LNPs reaching and remaining in the late endosome could explain such an increase in association between A1-25 and acidic organelles in the cell.

### Example 11: Endosomal Trafficking of Top LNP Formulations in Jurkats

The effects of S2, A1-25, and A1-50 on endosomal trafficking behavior were subsequently investigated. Specifically, three proteins that associate with various stages of the endosome were assessed: Rab5, Rab7, and Rab11. Rab5 tends to associate with early endosomes which provides insight on cell uptake of LNPs. Rab7 associates with the late endosome and has been previously shown to be the direct precursor stage to endosomal escape and functional delivery. Rab11 associates with the recycling endosome, which includes the exocytosis of endocytosed LNPs.

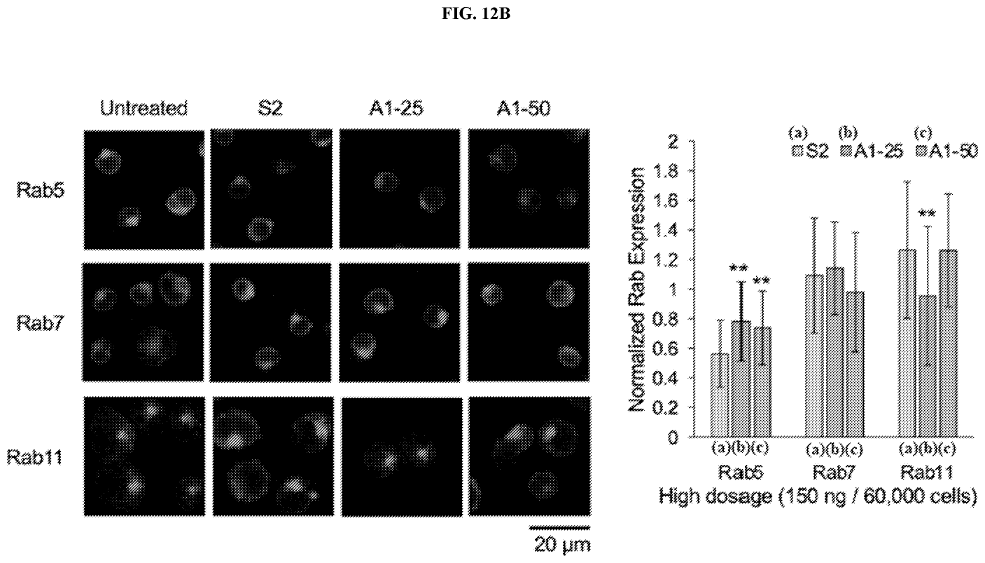

At both low and high doses, all three LNP formulations had reduced Rab5 expression as compared to untreated cells (FIG. 12A-12B). Without wishing to be bound by theory, this phenomenon may be explained by the transition of early endosomes to late endosomes through the introduction of exogenous materials (i.e., LNPs). However, at high doses, Rab5 expression in both A1-25 and A1-50 significantly increases, trending towards levels observed in untreated cells. Given that A1-25 and A1-50 both showed increases in functional delivery of mRNA to Jurkats in the initial screen described herein, it has been proposed that this increase in early endosome generation is the result of increased cellular uptake of LNPs rather than a reduction in early endosome progression to the late endosome. Without wishing to be bound by theory, the introduction of hydroxycholesterol molecules to the LNP formulations may cause morphological changes in the LNPs which impact cellular uptake.

All 3 formulations had increased Rab7 expression profiles at both high doses as compared to untreated cells. At low doses, A1-25 has significantly improved Rab7 expression as compared to S2. This is an expected result given that late endosomes are most closely associated with endosomal escape and A1-25 had significantly enhanced mRNA delivery in Jurkats compared to S2 in previous screens. However, at high doses, no significant difference was observed between the 3 formulations.

Regarding the Rab 11-associated recycling pathway, A1-25 and A1-50 produced significantly lower Rab11 expression at lower doses. In conjunction with functional delivery results, this decreased expression suggests that A1-25 and A1-50 have tend to reside in and escape the late endosome rather than being recycled out of the cell. At high doses, A1-25 maintains this trend, but A1-50 no longer significantly reduces Rab11 expression.

Taken together, the expression of these various endosomal markers suggests that 25% and 50% substitutions of hydroxycholesterol for unmodified cholesterol markedly improves functional delivery of mRNA to T cells through a combination of improved cellular uptake, increased generation of late endosomes, and reduced endosomal recycling.

### Example 12: In Vitro Evaluation of LNPs Comprising Cholesterol Analogs (i.e., Bile Acids)

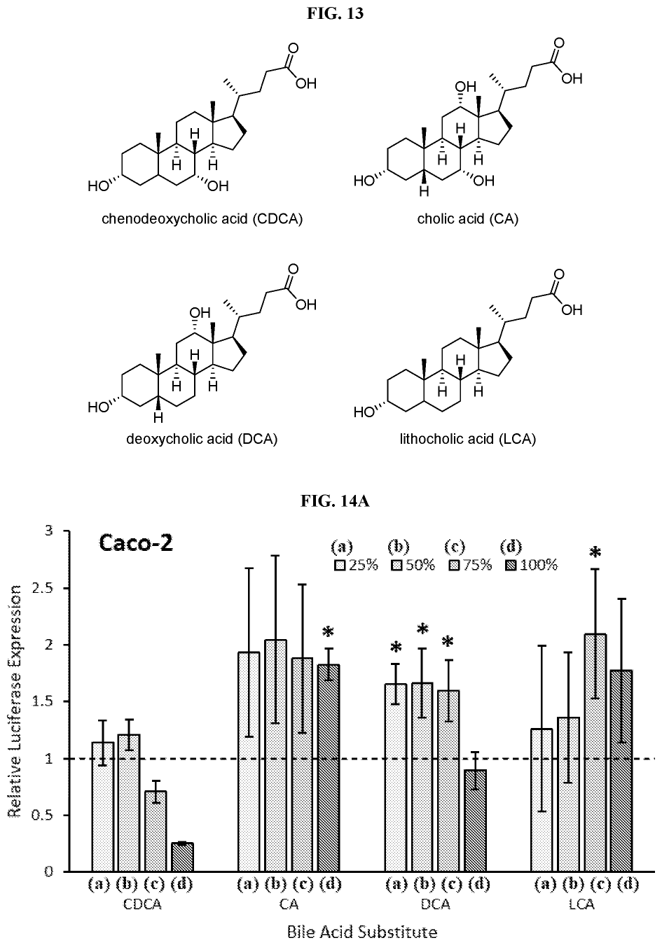

The present disclosure further provides exemplary data relating to in vitro analysis of LNP formulations comprising selected bile acids, including but not limited to chenodeoxycholic acid (CDCA), cholic acid (CA), deoxycholic acid (DCA), lithocholic acid (LCA), taurocholic acid, glycocholic acid, taurochenodeoxycholic acid, glycochenodeoxycholic acid (FIG. 13), wherein a portion of the cholesterol component of the LNP is substituted for a bile acid. LNP formulations comprising bile acid-cholesterol substitutions were prepared in a manner analogous to other LNPs described herein (Table 4).

TABLE 4
Selected Exemplary Bile Acid-Substituted LNP Formulations

| | | | | | PEG- |
|---|---|---|---|---|---|
| | Bile | | | | Conjugated |
| | Acid^{a} | Cholesterol | C14-494 | DOPE | Lipid |
| LNP | (mol %) | (mol %) | (mol %) | (mol %) | (mol %) |
| CDCA-25 | 11.625 | 34.875 | 35 | 16 | 2.5 |
| CDCA-50 | 23.25 | 23.25 | 35 | 16 | 2.5 |
| CDCA-75 | 34.875 | 11.625 | 35 | 16 | 2.5 |
| CDCA-100 | 46.5 | 0 | 35 | 16 | 2.5 |
| CA-25 | 11.625 | 34.875 | 35 | 16 | 2.5 |
| CA-50 | 23.25 | 23.25 | 35 | 16 | 2.5 |
| CA-75 | 34.875 | 11.625 | 35 | 16 | 2.5 |
| CA-100 | 46.5 | 0 | 35 | 16 | 2.5 |
| DCA-25 | 11.625 | 34.875 | 35 | 16 | 2.5 |
| DCA-50 | 23.25 | 23.25 | 35 | 16 | 2.5 |
| DCA-75 | 34.875 | 11.625 | 35 | 16 | 2.5 |
| DCA-100 | 46.5 | 0 | 35 | 16 | 2.5 |
| LCA-25 | 11.625 | 34.875 | 35 | 16 | 2.5 |
| LCA-50 | 23.25 | 23.25 | 35 | 16 | 2.5 |
| LCA-75 | 34.875 | 11.625 | 35 | 16 | 2.5 |
| LCA-100 | 46.5 | 0 | 35 | 16 | 2.5 |

^{a}Bile acid identity indicated in LNP title (e.g., CDCA-25 comprises bile acid CDCA).

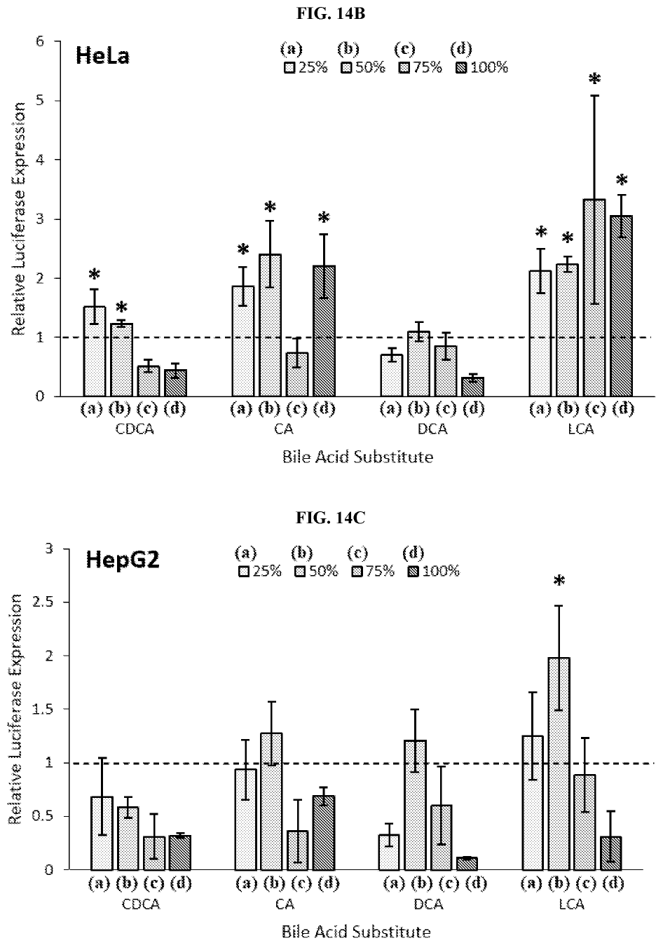

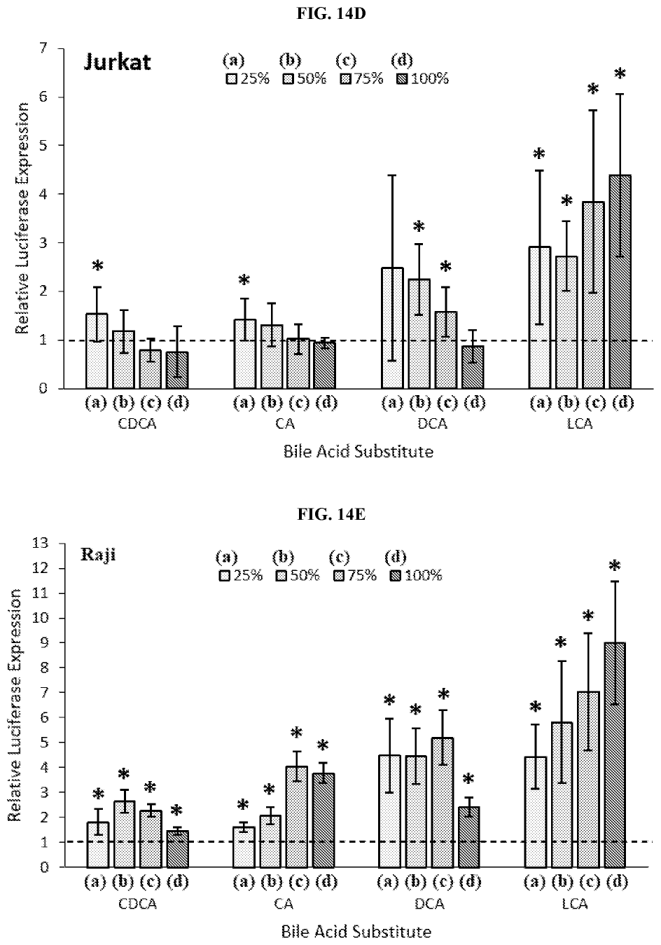

The bile acid-substituted LNPs described herein, wherein luciferase mRNA was encapsulated therein, were evaluated in vitro for delivery and/or expression of luciferase in Caco-2, HeLa, HepG2, Jurkat, and Raji cell lines (FIGS. 14A-14E). In certain embodiments, the bile-acid substituted LNPs of the present disclosure demonstrated superior results in epithelial cells and HeLa cells. In certain embodiments, the LNPs of the present disclosure demonstrated superior results in lymphocytes.

### Example 13: In Vivo Evaluation of LNPs Comprising Cholesterol Analogs (i.e., Bile Acids)

The present disclosure further provides exemplary data relating to in vivo analysis of LNP formulations comprising selected bile acids, wherein selected LNPs were identified in the in vitro evaluated described elsewhere herein for further evaluation (i.e., LNP CA-100, DCA-50, and LCA-75).

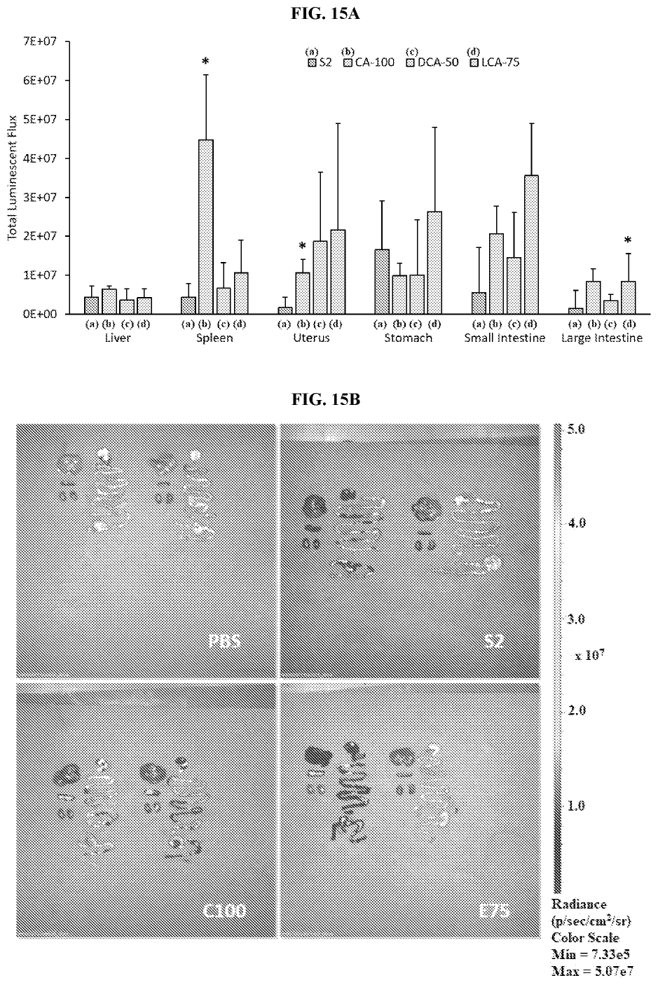

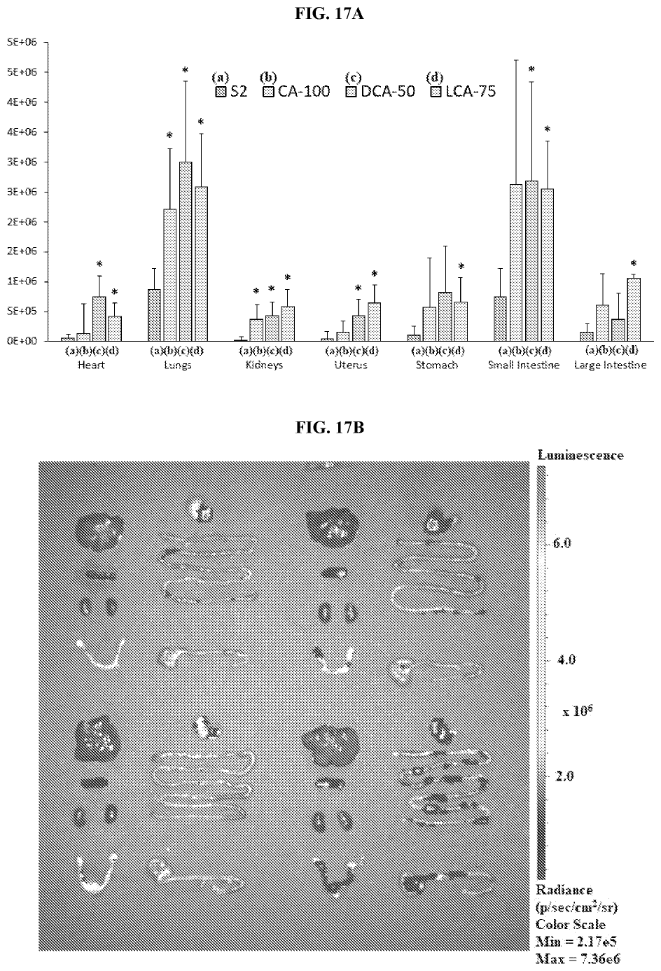

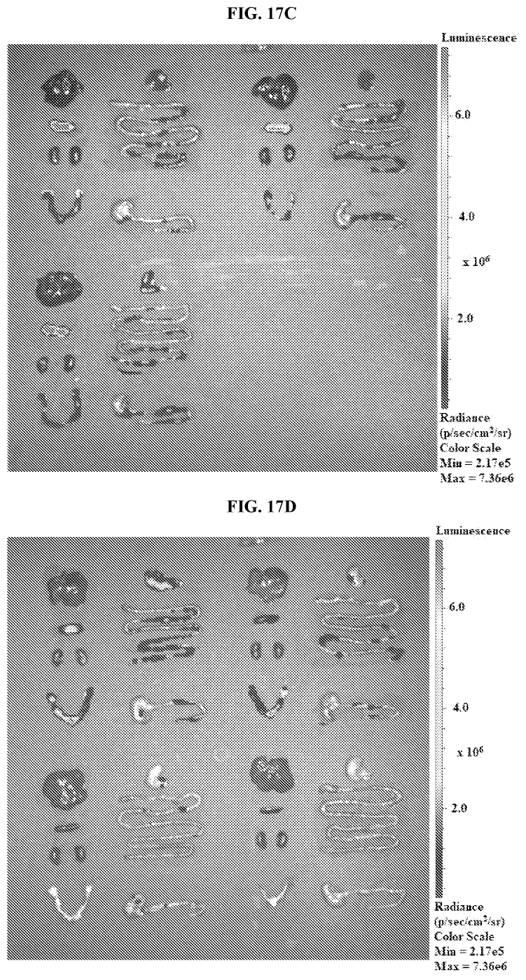

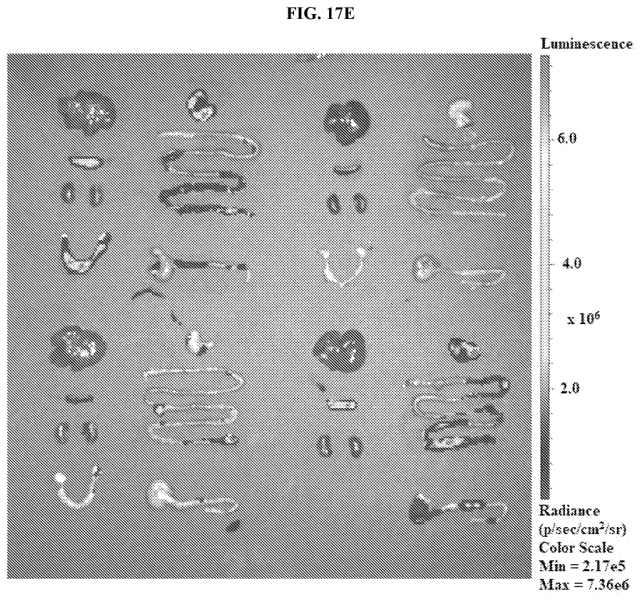

In certain embodiments, mice were intraperitoneally administered the LNPs of the present disclosure, wherein luciferase mRNA was encapsulated therein, and delivery and/or expression of mRNA in target organs was evaluated (FIGS. 15A-15B and FIGS. 17A-17E). In certain embodiments, Ca-100 significantly improves delivery to the spleen. In certain embodiments, LCA-75 significantly improves delivery to the small intestine. In certain embodiments, LNPs comprising bile acid-cholesterol substitution increase delivery to the small intestine and lung.

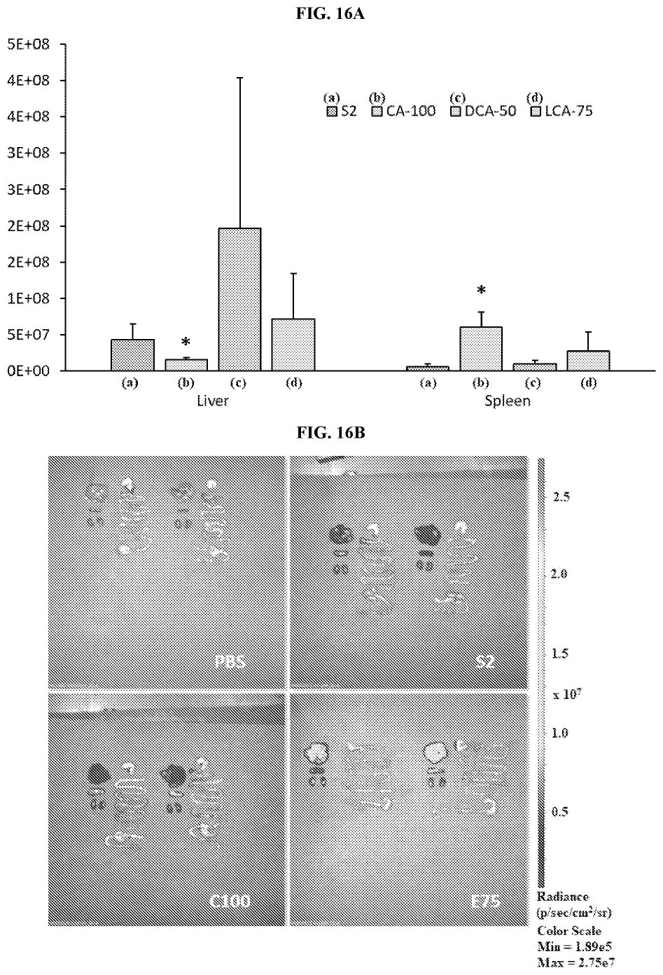

In certain embodiments, mice were intravenously administered the LNPs of the present disclosure, wherein luciferase mRNA was encapsulated therein, and delivery and/or expression of mRNA in target organs was evaluated (FIGS. 16A-16B).

In certain embodiments, CA-100, DCA-50, and LCA-75 increase systemic delivery to extrahepatic organs (e.g., lung and small intestine). In certain embodiments, CA-100 increases spleen delivery when administered intraperitoneally and/or intravenously.

### Enumerated Embodiments

The following exemplary embodiments are provided, the numbering of which is not to be construed as designating levels of importance:

Embodiment 1 provides a lipid nanoparticle (LNP) comprising:
(a) at least one ionizable lipid, wherein the ionizable lipid comprises about 10 mol % to about 50 mol % of the LNP;
(b) at least one helper lipid, wherein the helper lipid comprises about 10 mol % to about 45 mol % of the LNP;
(c) at least one selected from the group consisting of cholesterol and a cholesterol-substitute, wherein the combination of the cholesterol and cholesterol-substitute comprise about 5 mol % to about 50 mol % of the LNP; and
(d) at least one polyethylene glycol (PEG) or PEG-conjugated lipid, wherein the PEG or PEG conjugated lipid comprises about 0.5 mol % to about 12.5 mol % of the LNP.

Embodiment 2 provides the LNP of Embodiment 1, wherein the ionizable lipid is a compound of Formula (I), or a salt or solvate thereof:

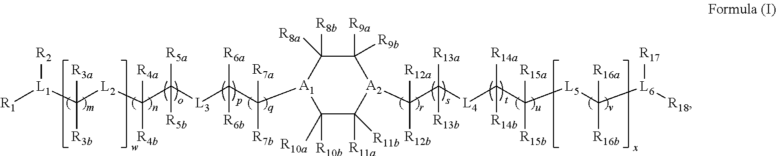

wherein:

A₁ and A₂ is independently selected from the group consisting of CH, N, and P;
L₁ and L₆ are each independently selected from the group consisting of CR₁₉ and N;
each occurrence of L₂ and L₅ is independently selected from the group consisting of —CH₂—, —CHR₁₉—, —O—, —NH—, and —NR₁₉—;
L₃ and L₄ are each independently selected from the group consisting of —CH₂—, —CHR₁₉—, —O—, —NH—, and —NR₁₉—;
each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, Rab, R₉ₐ, R_{9b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆ₐ, R_{16b}, R₁₇, R₁₈, and R₁₉ is independently selected from the group consisting of H, halogen, optionally substituted C₁-C₂₈ alkyl, optionally substituted C₃-C₁₂ cycloalkyl, —Y(R₂₀)_{z′}(R₂₁)_{z″}-(optionally substituted C₃-C₁₂ cycloalkyl, optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₈ alkenyl, optionally substituted C₅-C₁₂ cycloalkenyl, —Y(R₂₀)_{z′} (R₂₁)_{z″}-(optionally substituted C₅-C₁₂ cycloalkenyl), optionally substituted C₂-C₂₈ alkynyl, optionally substituted C₆-C₁₂ cycloalkynyl, —Y(R₂₀)_{z′}(R₂₁)_{z″}-(optionally substituted C₆-C₁₂ cycloalkynyl), optionally substituted C₆-C₁₀ aryl, —Y(R₂₀)_{z′}(R₂₁)_{z″}-(optionally substituted C₆-C₁₀ aryl), optionally substituted C₂-C₁₂ heteroaryl, —Y(R₂₀)_{z′}(R₂₁)_{z″}-(optionally substituted C₂-C₁₂ heteroaryl), C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —Y(R₂₀)_{z′}(R₂₁)_{z″}-ester, —Y(R₂₀)_{z′}(R₂₁)_{z″}, —NO₂, —CN, and sulfoxy,
   or two geminal substituents selected from R₃ₐ and R_{3b}, R₄ₐ and R_{4b}, R₅ₐ and R_{5b}, R₆ₐ, and R_{6b}, R₇ₐ and R_{7b}, R₈ₐ and R_{8b}, R₉ₐ and R_{9b}, R₁₀ₐ and R_{10b}, R₁₁ₐ and R_{11b}, R₁₂ₐ and R_{12b}, R₁₃ₐ and R_{13b}, R₁₄ₐ and R_{14b}, or R₁₅ₐ and R_{15b} can combine with the C atom to which they are bound to form C═O;
each occurrence of Y is independently selected from the group consisting of C, N, O, S, and P;
each occurrence of R₂₀ and R₂₁ is independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —NO₂, —CN, and sulfoxy,
   or R₂₀ and R₂₁ can combine with the Y atom to which they are bound to form a C═O);
each occurrence of z′ and z″ is independently 0, 1, or 2; and
each occurrence of m, n, o, p, q, r, s, t, u, v, w, and x are is independently 0, 1, 2; 3, 4, or 5.

Embodiment 3 provides the LNP of Embodiment 2, wherein the ionizable lipid of Formula (I) is selected from the group consisting of:

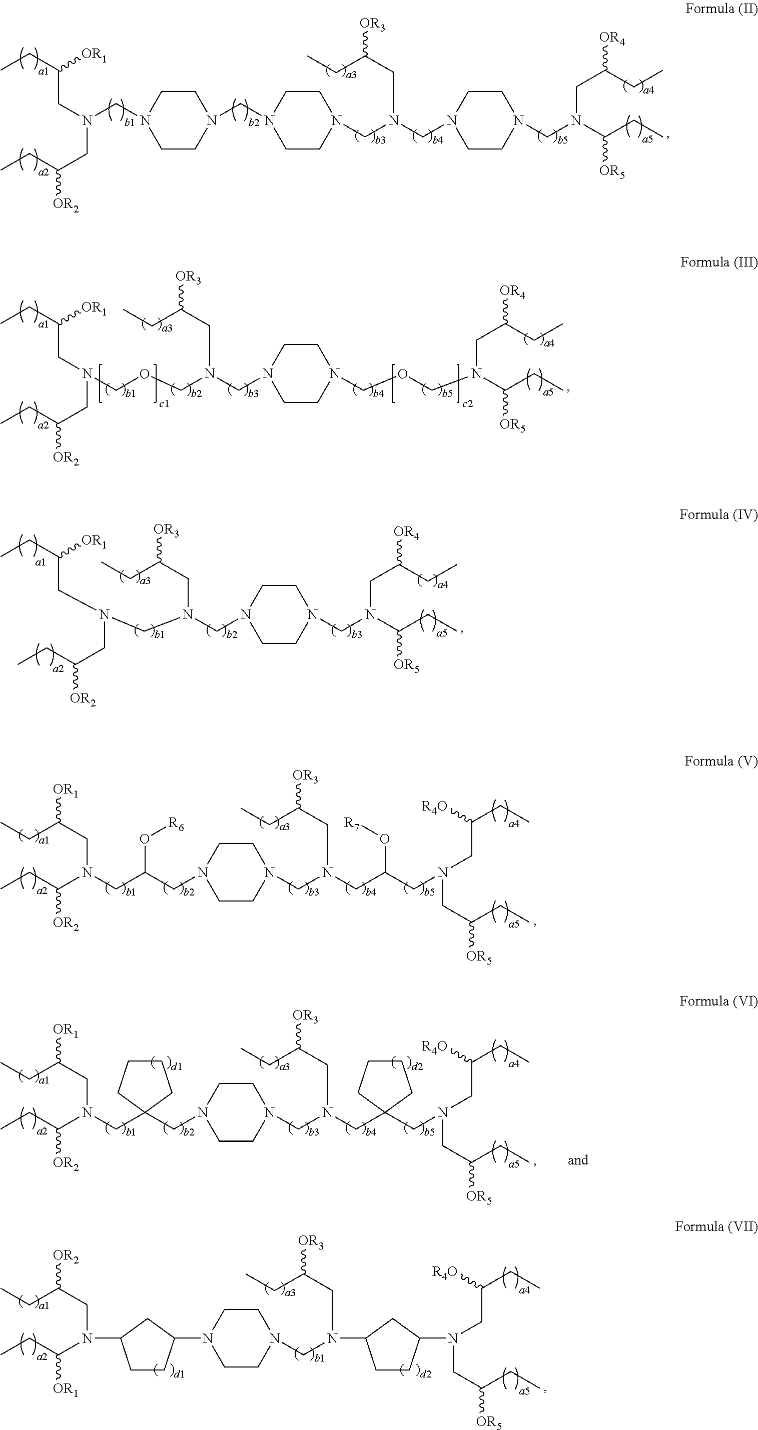

wherein:
R₁, R₂, R₃, R₄, R₅, R₆, and R₇ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester;
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25;
b¹, b², b³, b⁴, and b⁵ are each independently 0, 1, 2, 3, 4, or 5;
c¹ and c² are each independently 0, 1, 2, 3, 4, or 5; and
d¹ and d² are each independently 0, 1, 2, 3, 4, or 5.

Embodiment 4 provides the LNP of Embodiment 2, wherein the ionizable lipid of Formula (I) is selected from the group consisting of:

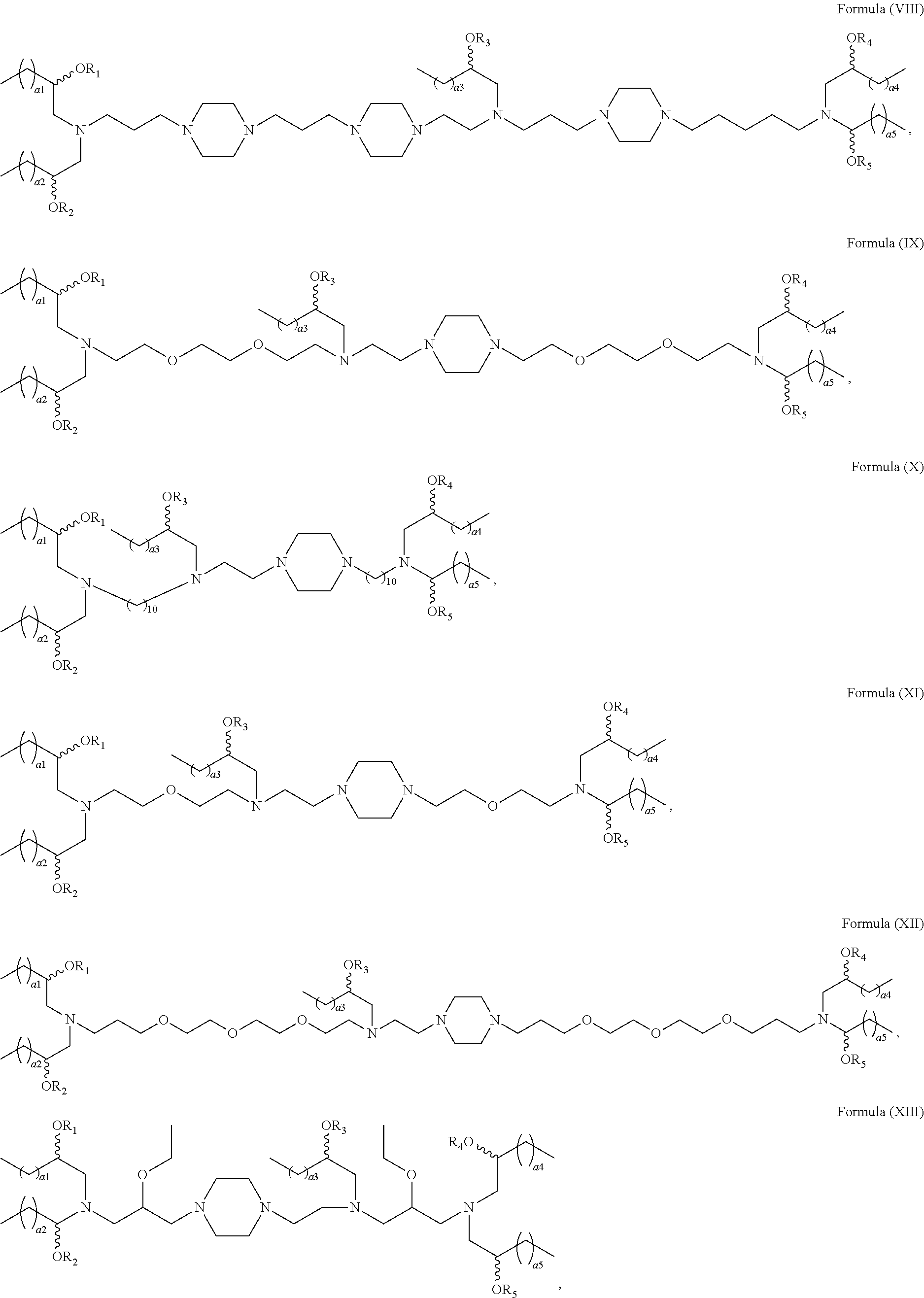

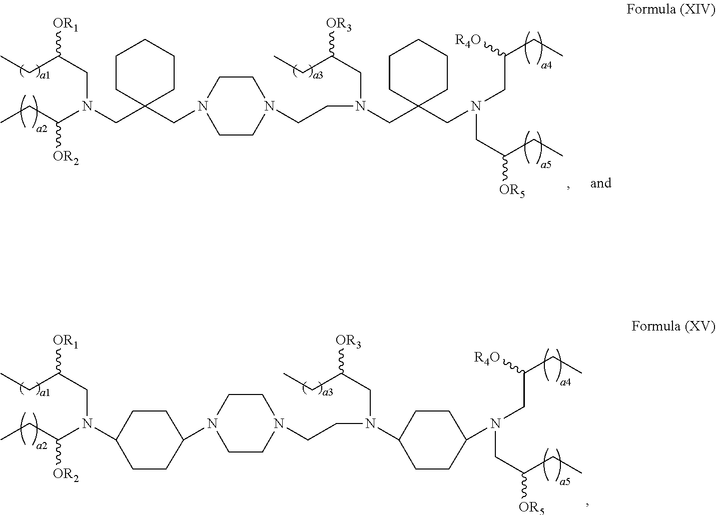

wherein:
R₁, R₂, R₃, R₄, and R₅ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester; and
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25.

Embodiment 5 provides the LNP of any one of Embodiments 2-4, wherein the ionizable lipid of Formula (I) comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol):

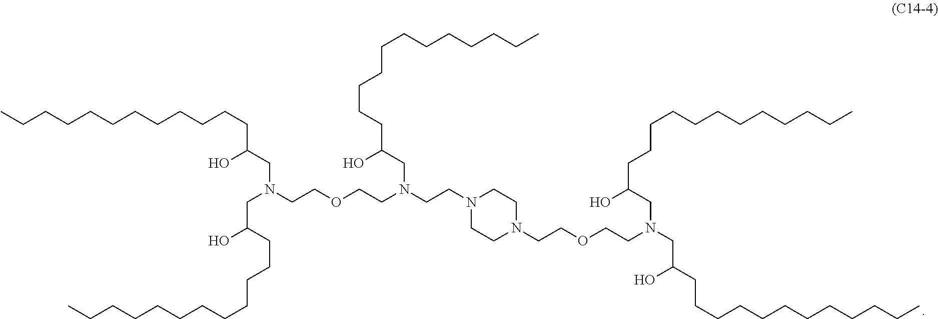 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

Embodiment 6 provides the LNP of any one of Embodiments 1-5, wherein the cholesterol-substitute is dexamethasone.

Embodiment 7 provides the LNP of Embodiment 6, wherein the cholesterol and cholesterol-substitute have a weight ratio selected from the group consisting of 9:1, 8:2, 7:3, and 5:5 (cholesterol:dexamethasone).

Embodiment 8 provides the LNP of Embodiment 6 or 7, wherein the LNP comprises at least one lipid selected from the group consisting of MC3 and C12-200.

Embodiment 9 provides the LNP of any one of Embodiments 6-8, wherein the helper lipid is 1,2-distearoyl-sn-glycero-3-phosphoethanolamine (DSPC).

Embodiment 10 provides the LNP of any one of Embodiments 6-9, wherein the PEG or PEG-conjugated lipid comprises 1,2-dimyristoyl-sn-glycero-3-phosphoethanolamine-N-(methoxy(polyethyleneglycol)-2000) (C14PEG-2000).

Embodiment 11 provides the LNP of any one of Embodiments 6-10, wherein the molar ratio of (a):(b):(c):(d) is about 50:10:38.5:1.5.

Embodiment 12 provides the LNP of any one of Embodiments 1-5, wherein the cholesterol-substitute is selected from the group consisting of a hydroxy substituted cholesterol, an epoxy substituted cholesterol, and a keto substituted cholesterol.

Embodiment 13 provides the LNP of any one of Embodiments 1-5 and 12, wherein the cholesterol-substitute is selected from the group consisting of 7-α-hydroxycholesterol, 7-β-hydroxycholesterol, 19-hydroxycholesterol, 20-(S)-hydroxycholesterol, 24-(S)-hydroxycholesterol, 25-hydroxycholesterol, 7-ketocholesterol, 5,6-epoxycholesterol, 38,5α,6β-trihydroxycholesterol, 4β-hydroxycholesterol, 27-hydroxycholesterol and 22-(R)-hydroxycholesterol.

Embodiment 14 provides the LNP of any one of Embodiments 1-5 and 12-13, wherein the cholesterol and cholesterol-substitute have a molar percentage ratio selected from the group consisting of about 50:50, 75:25, 87.5:12.5, and about 0:100 (cholesterol:cholesterol-substitute).

Embodiment 15 provides the LNP of any one of Embodiments 1-5 and 12-14, wherein the helper lipid is dioleoyl-phosphatidylethanolamine (DOPE).

Embodiment 16 provides the LNP of any one of Embodiments 1-5 and 12-15, wherein the PEG or PEG-conjugated lipid comprises 1,2-dimyristoyl-sn-glycero-3-phosphoethanolamine-N-(methoxy(polyethyleneglycol)-2000) (C14PEG-2000).

Embodiment 17 provides the LNP of any one of Embodiments 1-5 and 12-16, wherein the molar ratio of (a):(b):(c):(d) is about 30:16:46.5:2.5.

Embodiment 18 provides the LNP of Embodiment 17, wherein (c) comprises cholesterol and 7-α-hydroxycholesterol, wherein the cholesterol and 7α-hydroxycholesterol have a molar ratio selected from the group consisting of 50:50 and 75:25 (cholesterol: 7-α-hydroxycholesterol).

Embodiment 19 provides the LNP of any one of Embodiments 1-5, wherein the cholesterol-substitute is a carboxy-substituted cholesterol.

Embodiment 20 provides the LNP of any one of Embodiments 1-5 and 19, wherein the cholesterol-substitute is a bile acid.

Embodiment 21 provides the LNP of any one of Embodiments 1-5 and 19-20, wherein the cholesterol-substitute is selected from the group consisting of chenodeoxycholic acid (CDCA), cholic acid (CA), deoxycholic acid (DCA), lithocholic acid (LCA), taurocholic acid, glycocholic acid, taurochenodeoxycholic acid, and glycochenodeoxycholic acid.

Embodiment 22 provides the LNP of any one of Embodiments 1-5 and 19-21, wherein the cholesterol and cholesterol-substitute have a molar ratio selected from the group consisting of 25:100, 50:50, 75:25, and 100:0 (cholesterol-substitute:cholesterol).

Embodiment 23 provides the LNP of any one of Embodiments 1-5 and 19-22, wherein the helper lipid comprises dioleoyl-phosphatidylethanolamine (DOPE).

Embodiment 24 provides the LNP of any one of Embodiments 1-5 and 19-23, wherein the PEG or PEG-conjugated lipid comprises 1,2-dimyristoyl-sn-glycero-3-phosphoethanolamine-N-(methoxy(polyethyleneglycol)-2000) (C14PEG-2000).

Embodiment 25 provides the LNP of any one of Embodiments 1-5 and 19-24, wherein the molar ratio of (a):(b):(c):(d) is about 35:16:46.5:2.5.

Embodiment 26 provides the LNP of any one of Embodiments 1-25, wherein the LNP further comprises at least one selected from the group consisting of a nucleic acid molecule and a therapeutic agent.

Embodiment 27 provides the LNP of any one of Embodiments 1-26, wherein the LNP further comprises at least one agent selected from the group consisting of an mRNA, a siRNA, a microRNA, a CRISPR-Cas9, a small molecule, a protein, and an antibody.

Embodiment 28 provides the LNP of Embodiment 26, wherein the LNP comprises a nucleic acid molecule.

Embodiment 29 provides the LNP of Embodiment 28, wherein the nucleic acid molecule is a DNA molecule or an RNA molecule.

Embodiment 30 provides the LNP of Embodiment 28 or 29, wherein the nucleic acid molecule is selected from the group consisting of cDNA, mRNA, miRNA, siRNA, modified RNA, antagomir, antisense molecule, and a targeted nucleic acid, or any combination thereof.

Embodiment 31 provides the LNP of Embodiment 28, wherein the nucleic acid molecule encodes a chimeric antigen receptor (CAR).

Embodiment 32 provides the LNP of Embodiment 31, wherein the CAR is specific for binding to a surface antigen of a pathogenic cell or a tumor cell.

Embodiment 33 provides the LNP of any one of Embodiments 1-32, wherein the LNP further comprises a targeting domain specific for binding to a target cell of interest.

Embodiment 34 provides the LNP of Embodiment 33, wherein the target cell is selected from the group consisting of a peripheral blood mononuclear cell and an immune cell.

Embodiment 35 provides the LNP of any one of Embodiments 1-34, wherein the LNP comprises an immune cell targeting domain specific for binding to a T cell.

Embodiment 36 provides the LNP of Embodiment 35, wherein the targeting domain specifically binds to at least one surface molecule selected from the group consisting of CD1, CD2, CD3, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7.

Embodiment 37 provides a pharmaceutical composition comprising the LNP of any one of Embodiments 1-36 and a pharmaceutically acceptable carrier.

Embodiment 38 provides the pharmaceutical composition of Embodiment 37, wherein the pharmaceutical composition further comprises an adjuvant.

Embodiment 39 provides the pharmaceutical composition of Embodiment 37 or 38, wherein the pharmaceutical composition is a vaccine.

Embodiment 40 provides a method of delivering at least one selected from the group consisting of a nucleic acid molecule and a therapeutic agent to a target cell in a subject in need thereof, the method comprising administering to the subject a therapeutically effectively amount of at least one LNP of any one of Embodiments 1-36 and/or the pharmaceutical composition of any one of Embodiments 37-39.

Embodiment 41 provides the method of Embodiment 40, wherein the therapeutic agent is at least one selected from the group consisting of an mRNA, a siRNA, a microRNA, a CRISPR-Cas9, a small molecule, a protein, and an antibody.

Embodiment 42 provides the method of Embodiment 40, wherein the nucleic acid molecule is at least one selected from the group consisting of a DNA molecule and an RNA molecule.

Embodiment 43 provides the method of Embodiment 40, wherein the nucleic acid molecule is at least one selected from the group consisting of cDNA, mRNA, miRNA, siRNA, antagomir, antisense molecule, and a targeted nucleic acid.

Embodiment 44 provides the method of Embodiment 40, wherein the nucleic acid molecule encodes a chimeric antigen receptor (CAR).

Embodiment 45 provides the method of Embodiment 44, wherein the CAR is specific for binding to a surface antigen of a pathogenic cell or tumor cell.

Embodiment 46 provides the method of any one of Embodiments 40-45, wherein the target cell is selected from the group consisting of a stem cell, a peripheral blood mononuclear cell, and an immune cell.

Embodiment 47 provides the method of Embodiment 45 or 46, wherein the CAR comprises a cell targeting domain specific for binding to a T cell.

Embodiment 48 provides the method of Embodiment 47, wherein the cell targeting domain is specific for binding to at least one selected from the group consisting of CD1, CD2, CD3, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7.

Embodiment 49 provides the method of any one of Embodiments 40-48, wherein the LNP or pharmaceutical composition thereof further comprises an adjuvant.

Embodiment 50 provides the method of any one of Embodiments 40-49, wherein the nucleic acid molecule and/or therapeutic agent is at least partially encapsulated within the LNP.

Embodiment 51 provides the method of any one of Embodiments 40-50, wherein the method treats, prevents, and/or ameliorates at least one selected from the group consisting of a viral infection, a bacterial infection, a fungal infection, a parasitic infection, cancer, or a disease or disorder associated with cancer.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A lipid nanoparticle (LNP) comprising:
(a) at least one ionizable lipid, wherein the ionizable lipid comprises about 10 mol % to about 50 mol % of the LNP;
(b) at least one helper lipid, wherein the helper lipid comprises about 10 mol % to about 45 mol % of the LNP;
(c) at least one selected from the group consisting of cholesterol or and a cholesterol-substitute, wherein the combination of the cholesterol and cholesterol-substitute comprise about 5 mol % to about 50 mol % of the LNP; and
(d) at least one polyethylene glycol (PEG) or PEG-conjugated lipid, wherein the PEG or PEG conjugated lipid comprises about 0.5 mol % to about 12.5 mol % of the LNP.

2. The LNP of claim 1, wherein the ionizable lipid is a compound of Formula (I), or a salt or solvate thereof: 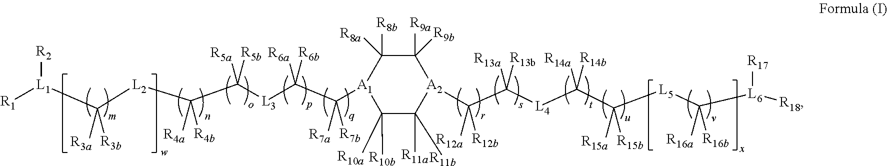
wherein:
A₁ and A₂ is independently selected from the group consisting of CH, N, and P;
Li and L₆ are each independently selected from the group consisting of CR₁₉ and N;
each occurrence of L₂ and L₅ is independently selected from the group consisting of —CH₂—, —CHR₁₉—, —O—, —NH—, and —NR₁₉—;
L₃ and L₄ are each independently selected from the group consisting of —CH₂—, —CHR₁₉—, —O—, —NH—, and —NR₁₉—;
each occurrence of R₁, R₂, R₃ₐ, R_{3b}, R₄ₐ, R_{4b}, R₅ₐ, R_{5b}, R₆ₐ, R_{6b}, R₇ₐ, R_{7b}, R₈ₐ, R_{8b}, R₉ₐ, R_{6b}, R₁₀ₐ, R_{10b}, R₁₁ₐ, R_{11b}, R₁₂ₐ, R_{12b}, R₁₃ₐ, R_{13b}, R₁₄ₐ, R_{14b}, R₁₅ₐ, R_{15b}, R₁₆ₐ, R_{16b}, R₁₇, R₁₈, and R₁₉ is independently selected from the group consisting of H, halogen, optionally substituted C₁-C₂₈ alkyl, optionally substituted C₃-C₁₂ cycloalkyl, —Y(R₂₀)_{z′}(R₂₁)_{z″}-(optionally substituted C₃-C₁₂ cycloalkyl, optionally substituted C₂-C₁₂ heterocycloalkyl, —Y(R₂₀)_{z′}(R₂₁)_{z″}-(optionally substituted C₂-C₁₂ heterocycloalkyl), optionally substituted C₂-C₂₈ alkenyl, optionally substituted C₅-C₁₂ cycloalkenyl, —Y(R₂₀)_{z′}(R₂₁)_{z″}-(optionally substituted C₅-C₁₂ cycloalkenyl), optionally substituted C₂-C₂₈ alkynyl, optionally substituted C₆-C₁₂ cycloalkynyl, —Y(R₂₀)_{z′}(R₂₁)_{z″}-(optionally substituted C₆-C₁₂ cycloalkynyl), optionally substituted C₆-C₁₀ aryl, —Y(R₂₀)_{z′}(R₂₁)_{z″}-(optionally substituted C₆-C₁₀ aryl), optionally substituted C₂-C₁₂ heteroaryl, —Y(R₂₀)_{z′}(R₂₁)_{z″}-(optionally substituted C₂-C₁₂ heteroaryl), C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —Y(R₂₀)_{z′}(R₂₁)_{z″}-ester, —Y(R₂₀)_{z′}(R₂₁)_{z″}, —NO₂, —CN, and sulfoxy,
or two geminal substituents selected from R₃ₐ and R_{3b}, R₄ₐ and R_{4b}, R₅ₐ and R_{5b}, R₆ₐ, and R_{6b}, R₇ₐ and R_{7b}, R₈ₐ and R_{8b}, R₉ₐ and R_{6b}, R₁₀ₐ and R_{10b}, R₁₁ₐ and R_{11b}, R₁₂ₐ and R_{12b}, R₁₃ₐ and R_{13b}, R₁₄ₐ and R_{14b}, or R₁₅ₐ and R_{15b} can combine with the C atom to which they are bound to form C═O;
each occurrence of Y is independently selected from the group consisting of C, N, O, S, and P;
each occurrence of R₂₀ and R₂₁ is independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, ester, —NO₂, —CN, and sulfoxy,
or R₂₀ and R₂₁ can combine with the Y atom to which they are bound to form a C═O);
each occurrence of z′ and z″ is independently 0, 1, or 2; and
each occurrence of m, n, o, p, q, r, s, t, u, v, w, and x are is independently 0, 1, 2; 3, 4, or 5.

3. The LNP of claim 2, wherein the ionizable lipid of Formula (I) is selected from the group consisting of: 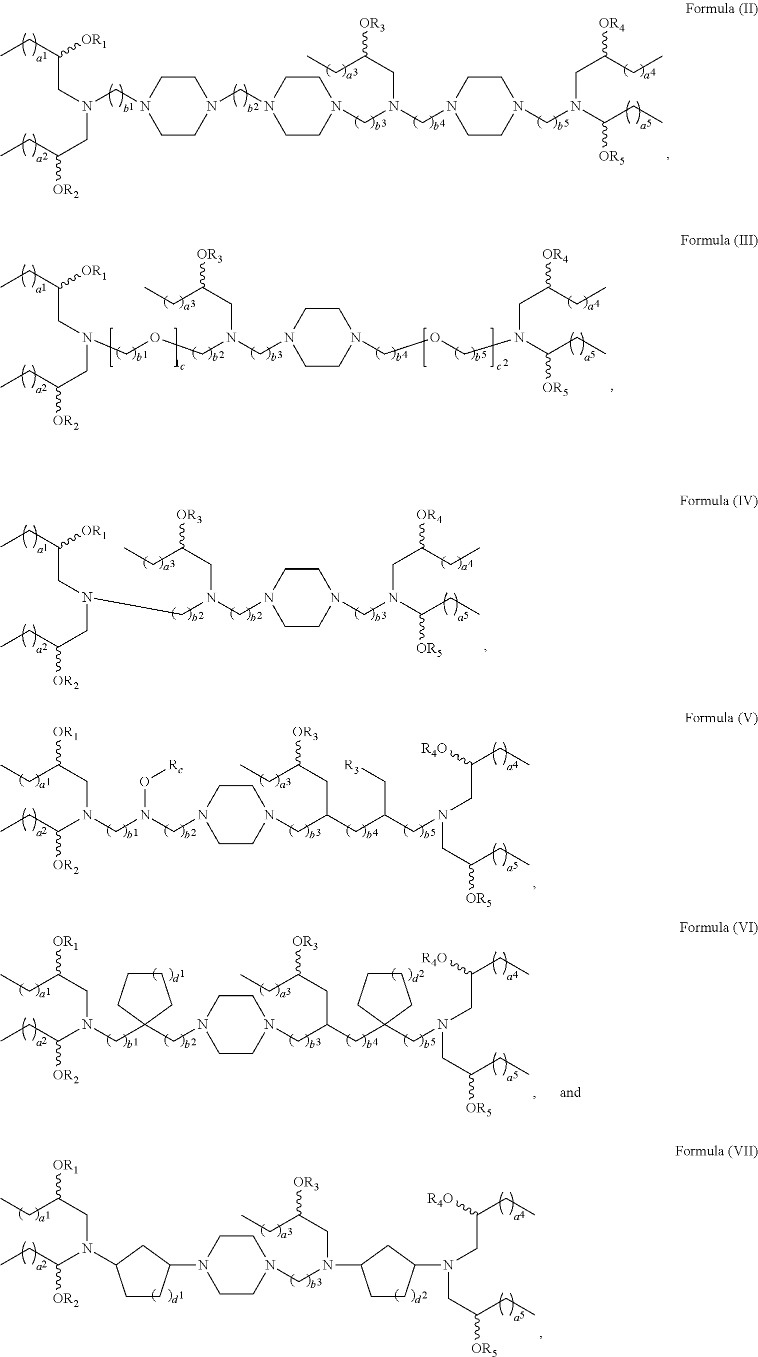
wherein:
R₁, R₂, R₃, R₄, R₅, R₆, and R₇ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester;
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25;
b¹, b², b³, b⁴, and b⁵ are each independently 0, 1, 2, 3, 4, or 5;
c¹ and c² are each independently 0, 1, 2, 3, 4, or 5; and
d¹ and d² are each independently 0, 1, 2, 3, 4, or 5.

4. The LNP of claim 2, wherein the ionizable lipid of Formula (I) is selected from the group consisting of: 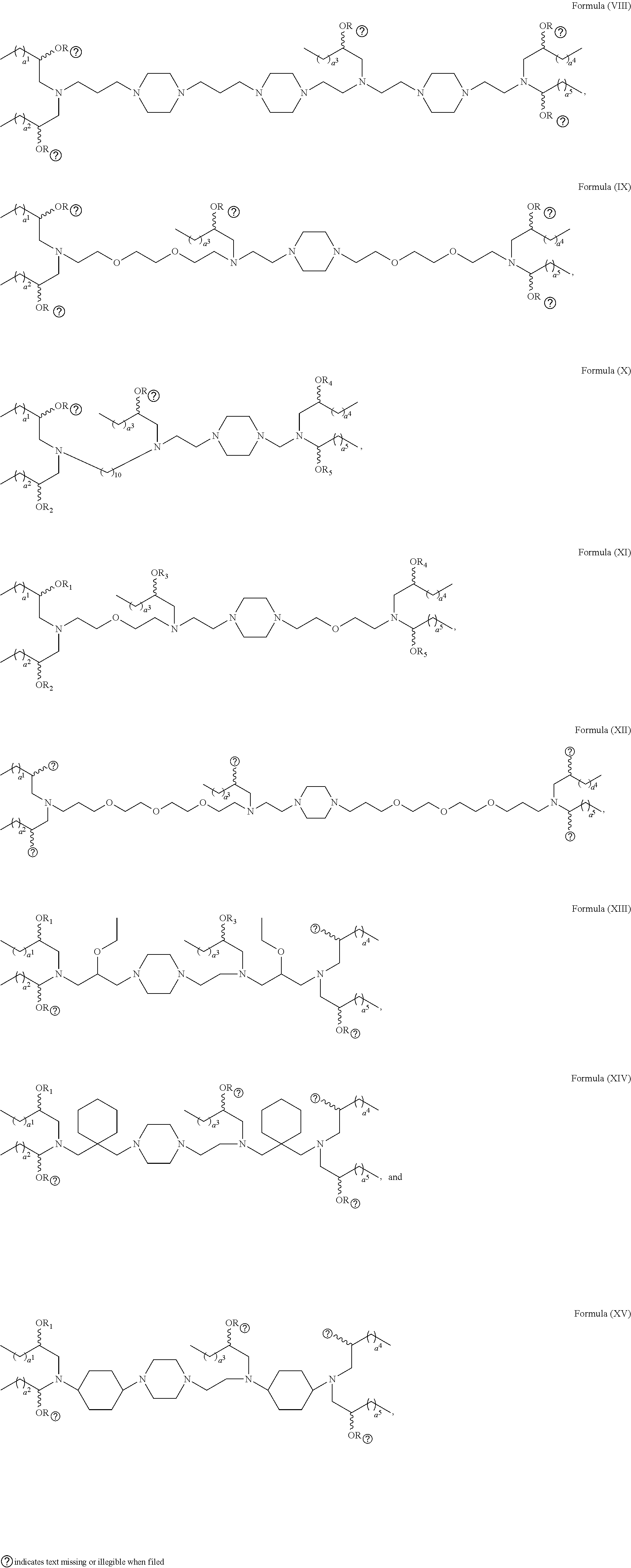
wherein:
R₁, R₂, R₃, R₄, and R₅ are each independently selected from the group consisting of H, halogen, optionally substituted C₁-C₂₈ alkyl, optionally substituted C₃-C₁₂ cycloalkyl, optionally substituted C₂-C₁₂ heterocycloalkyl, optionally substituted C₂-C₂₈ alkenyl, optionally substituted C₅-C₁₂ cycloalkenyl, optionally substituted C₂-C₂₈ alkynyl, optionally substituted C₆-C₁₂ cycloalkynyl, optionally substituted C₆-C₁₀ aryl, optionally substituted C₂-C₁₂ heteroaryl, C₁-C₂₈ alkoxycarbonyl, linear C₁-C₂₈ alkoxycarbonyl, branched C₁-C₂₈ alkoxycarbonyl, C(═O) NH₂, NH₂, C₁-C₂₈ aminoalkyl, C₂-C₂₈ aminoalkenyl, C₂-C₂₈ aminoalkynyl, C₆-C₁₀ aminoaryl, aminoacetate, acyl, OH, C₁-C₂₈ hydroxyalkyl, C₂-C₂₈ hydroxyalkenyl, C₂-C₂₈ hydroxyalkynyl, C₆-C₁₀ hydroxyaryl, C₁-C₂₈ alkoxy, carboxyl, carboxylate, and ester; and
a¹, a², a³, a⁴, and a⁵ are each independently 0, 1, 2, 3, 4, 5, 6, 7, 8, 9, 10, 11, 12, 13, 14, 15, 16, 17, 18, 19, 20, 21, 22, 23, 24, or 25.

5. The LNP of claim 2, wherein the ionizable lipid of Formula (I) comprises 1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol):

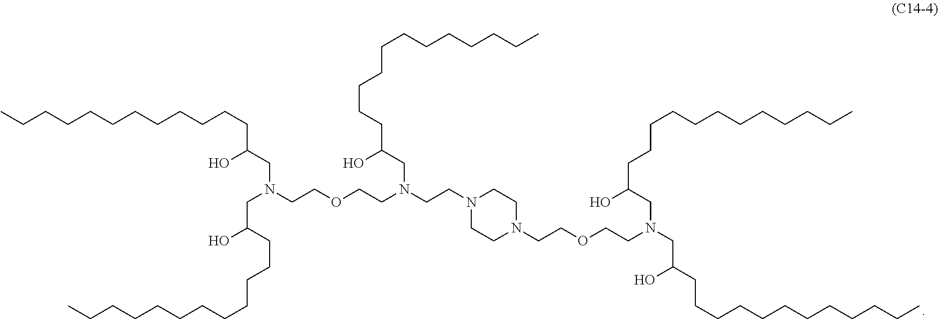 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

6. The LNP of claim 1, wherein at least one of the following applies:
(i) the cholesterol-substitute is dexamethasone, optionally wherein the cholesterol and cholesterol-substitute have a weight ratio selected from the group consisting of 9:1, 8:2, 7:3, and 5:5 (cholesterol:dexamethasone);
(ii) the LNP comprises at least one lipid selected from the group consisting of MC3 and C12-200;
(iii) the helper lipid is 1,2-distearoyl-sn-glycero-3-phosphoethanolamine (DSPC);
(iv) the PEG or PEG-conjugated lipid comprises 1,2-dimyristoyl-sn-glycero-3-phosphoethanolamine-N-(methoxy(polyethyleneglycol)-2000) (C14PEG-2000); and
(v) the molar ratio of (a):(b):(c):(d) is about 50:10:38.5:1.5.

7-11. (canceled)

12. The LNP of claim 1, wherein at least one of the following applies:
(i) the cholesterol-substitute is selected from the group consisting of a hydroxy substituted cholesterol, an epoxy substituted cholesterol, and a keto substituted cholesterol,
optionally wherein the cholesterol-substitute is selected from the group consisting of 7-α-hydroxycholesterol, 7-β-hydroxycholesterol, 19-hydroxycholesterol, 20-(S)-hydroxycholesterol, 24-(S)-hydroxycholesterol, 25-hydroxycholesterol, 7-ketocholesterol, 5,6-epoxycholesterol, 3β,5α,6β-trihydroxycholesterol, 4β-hydroxycholesterol, 27-hydroxycholesterol and 22-(R)-hydroxycholesterol, and optionally wherein (c) comprises cholesterol and 7-α-hydroxycholesterol, wherein the cholesterol and 7α-hydroxycholesterol have a molar ratio selected from the group consisting of 50:50 and 75:25 (cholesterol: 7-α-hydroxycholesterol);
(ii) the cholesterol and cholesterol-substitute have a molar percentage ratio selected from the group consisting of about 50:50, 75:25, 87.5:12.5, and about 0:100 (cholesterol:cholesterol-substitute);
(iii) the helper lipid is dioleoyl-phosphatidylethanolamine (DOPE);
(iv) the PEG or PEG-conjugated lipid comprises 1,2-dimyristoyl-sn-glycero-3-phosphoethanolamine-N-(methoxy(polyethyleneglycol)-2000) (C14PEG-2000); and
(v) the molar ratio of (a):(b):(c):(d) is about 30:16:46.5:2.5.

13-18. (canceled)

19. The LNP of claim 1, wherein at least one of the following applies:
(i) the cholesterol-substitute is a carboxy-substituted cholesterol;
(ii) the cholesterol-substitute is a bile acid;
(iii) the cholesterol-substitute is selected from the group consisting of chenodeoxycholic acid (CDCA), cholic acid (CA), deoxycholic acid (DCA), lithocholic acid (LCA), taurocholic acid, glycocholic acid, taurochenodeoxycholic acid, and glycochenodeoxycholic acid;
(iv) the cholesterol and cholesterol-substitute have a molar ratio selected from the group consisting of 25:100, 50:50, 75:25, and 100:0 (cholesterol-substitute:cholesterol);
(v) the helper lipid comprises dioleoyl-phosphatidylethanolamine (DOPE);
(vi) the PEG or PEG-conjugated lipid comprises 1,2-dimyristoyl-sn-glycero-3-phosphoethanolamine-N-(methoxy(polyethyleneglycol)-2000) (C14PEG-2000); and
(vii) the molar ratio of (a):(b):(c):(d) is about 35:16:46.5:2.5.

20-25. (canceled)

26. The LNP of claim 1, wherein the LNP further comprises at least one of the following:
(i) at least one selected from the group consisting of a nucleic acid molecule and a therapeutic agent; and
(ii) at least one agent selected from the group consisting of an mRNA, a siRNA, a microRNA, a CRISPR-Cas9, a small molecule, a protein, and an antibody.

27. (canceled)

28. The LNP of claim 26, wherein the LNP comprises a nucleic acid molecule, optionally wherein the nucleic acid molecule is a DNA molecule or RNA molecule.

29. (canceled)

30. The LNP of claim 28 or 29, wherein at least one of the following applies:
(i) the nucleic acid molecule is selected from the group consisting of cDNA, mRNA, miRNA, siRNA, modified RNA, antagomir, antisense molecule, and a targeted nucleic acid, or any combination thereof;
(ii) the nucleic acid molecule encodes a chimeric antigen receptor (CAR), optionally wherein the CAR is specific for binding to a surface antigen of a pathogenic cell or a tumor cell.

31-32. (canceled)

33. The LNP of claim 1, wherein at least one of the following applies:
(i) the LNP further comprises a targeting domain specific for binding to a target cell of interest, optionally wherein the target cell is selected from the group consisting of a peripheral blood mononuclear cell and an immune cell; and
(ii) the LNP comprises an immune cell targeting domain specific for binding to a T cell,
optionally wherein the targeting domain specifically binds to at least one surface molecule selected from the group consisting of CD1, CD2, CD3, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7.

34-36. (canceled)

37. A pharmaceutical composition comprising the LNP of claim 1 and a pharmaceutically acceptable carrier, optionally wherein at least one of the following applies:
(i) the pharmaceutical composition further comprises an adjuvant; and
(ii) the pharmaceutical composition is a vaccine.

38-39. (canceled)

40. A method of delivering at least one selected from the group consisting of a nucleic acid molecule and a therapeutic agent to a target cell in a subject in need thereof, the method comprising administering to the subject a therapeutically effectively amount of at least one LNP of claim 1, or a pharmaceutical composition thereof.

41. The method of claim 40, wherein the therapeutic agent is at least one selected from the group consisting of an mRNA, a siRNA, a microRNA, a CRISPR-Cas9, a small molecule, a protein, and an antibody.

42. The method of claim 40, wherein at least one of the following applies:
(i) the nucleic acid molecule is at least one selected from the group consisting of a DNA molecule and an RNA molecule;
(ii) the nucleic acid molecule is at least one selected from the group consisting of cDNA, mRNA, miRNA, siRNA, antagomir, antisense molecule, and a targeted nucleic acid; and
(iii) the nucleic acid molecule encodes a chimeric antigen receptor (CAR), optionally wherein the CAR is specific for binding to a surface antigen of a pathogenic cell or tumor cell.

43-45. (canceled)

46. The method of claim 40, wherein the target cell is selected from the group consisting of a stem cell, a peripheral blood mononuclear cell, and an immune cell.

47. The method of claim 42, wherein the CAR comprises a cell targeting domain specific for binding to a T cell,
optionally wherein the cell targeting domain is specific for binding to at least one selected from the group consisting of CD1, CD2, CD3, CD5, CD7, CD8, CD16, CD25, CD26, CD27. CD28, CD30, CD38, CD39, CD40L, CD44, CD45, CD62L, CD69, CD73, CD80, CD83, CD86. CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7.

48. (canceled)

49. The method of claim 40, wherein at least one of the following applies:
(i) the LNP or pharmaceutical composition thereof further comprises an adjuvant; and
(ii) the nucleic acid molecule and/or therapeutic agent is at least partially encapsulated within the LNP.

50. (canceled)

51. The method of claim 40, wherein the method treats, prevents, or ameliorates at least one selected from the group consisting of a viral infection, a bacterial infection, a fungal infection, a parasitic infection, cancer, or a disease or disorder associated with cancer.
